(19) United States
(12) Patent Application Publication
(10) Pub. No.: US 2010/0069340 A1
(43) Pub. Date: Mar. 18, 2010

(54) PHARMACEUTICAL COMPOSITIONS OF AN SRC KINASE INHIBITOR AND AN AROMATASE INHIBITOR

(75) Inventors: Charles Michael Zacharchuk, Westford, MA (US); Frank Charles Boschelli, New City, NY (US); Jennifer Michele Golas, Hewitt, NJ (US); Kim Timothy Arndt, Mountainside, NJ (US)

Correspondence Address: WYETH LLC;PATENT LAW GROUP, 5 GIRALDA FARMS, MADISON, NJ, 07940, US

(73) Assignee: Wyeth, Madison, NJ (US)

(21) Appl. No.: 12/557,668

(22) Filed: Sep. 11, 2009

Related U.S. Application Data
(60) Provisional application No. 61/096,155, filed on Sep. 11, 2008.
(60) Provisional application No. 61/166,387, filed on Apr. 3, 2009.

(51) Int. Cl. A61K 31/565; A61K 31/496; A61K 31/4709; A61K 31/4196; A61K 31/138; A61P 9/00; A61P 35/00

(52) U.S. Cl. 514/171; 514/253.06; 514/313; 514/252.19; 514/383; 514/648

(57) ABSTRACT

This invention is directed to a pharmaceutical composition comprising an Src kinase inhibitor and an aromatase inhibitor, and to the use of a combination of an Src kinase inhibitor and an aromatase inhibitor in treating abnormal cell proliferation and abnormal angiogenesis associated with cancer, including breast cancer.

### FIELD OF THE INVENTION

This invention relates to pharmaceutically acceptable compositions comprising an Src kinase inhibitor and an aromatase inhibitor, and to the use of a combination of an Src kinase inhibitor and an aromatase inhibitor for treating diseases characterized by abnormal cell proliferation and/or abnormal angiogenesis.

### BACKGROUND OF THE INVENTION

Tyrosine kinase enzymes may be divided into two groups: receptor tyrosine kinases and non-receptor tyrosine kinases. About 90 tyrosine kinases have been identified in the human genome, of which about 60 are of the receptor type and about 30 are of the non-receptor type. These can be categorised into 20 receptor tyrosine kinase sub-families according to the families of growth factors that they bind to, and into 10 non-receptor tyrosine kinase sub-families, as described by Robinson et al, in Oncogene, 2000, 19, 5548-5557. Src-family kinases are a group of 9 cytoplasmic (ie, non-receptor) tyrosine kinases defined by sequence similarity and by function. Three (Src, Yes, and FynB) are widely expressed in mammalian tissues; the 6 other members are predominantly expressed in hematopoietic cells: Lck, Lyn, FynT, Fgr, Hck, and Blk. The mechanisms by which Src becomes hyperactivated and promotes mitogenesis, survival, disaggregation and migration, G-protein signaling, and angiogenesis in human cancer have been extensively reviewed, as described by Summy J M, Gallick G E, “Src family kinases in tumor progression and metastasis,” in Cancer Metastasis Rev 2003; 22:337-58, and by Benati D, Baldari C T, “SRC family kinases as potential therapeutic targets for malignancies and immunological disorders,” in Curr Med Chem; 2008:1154-65. An association between c-Src and ERα levels has been reported, as described by Chu I, Arnaout A, Loiseau S, et al, “Src promotes estrogen-dependent estrogen receptor alpha proteolysis in human breast cancer,” in Journal of Clinical Investigations 2007; 117:2205-15. Estrogen drives both transcriptional activation and proteolysis of ERα. Estrogen-ERα binding promotes a rapid and transient interaction of ERα with cellular Src (c-SRc), binding to Shc, and Ras-MAPK activation, as described by Song R X-D, Zhang Z, Santen R J., “Estrogen rapid activation via protein complex formation involving ER and Src,” in Trends Endocrinology Metabolism 2005; 16(8):347-53; Ballare C, Uhrig M, Bechtold T, et al., “Two domains of the progesterone receptor interact with the estrogen receptor and are required for progesterone activation of the c-Src/Erk pathway in mammalian cells.,” in: Molecular Cell Biology 2003:1994-2008; Migliaccio A, et al., “Steroid-induced androgen receptor-oestradiol receptor beta-Src complex triggers prostate cancer cell proliferation,” in EMBO Journal 2000; 19:5406-17; and Wong C W, McNally C, Nickbarg E, et al., “Estrogen receptor-interacting protein that modulates its nongenomic activity-crosstalk with Src/Erk phosphorylation cascade,” in Proc Natl Acad Sci USA 2002; 99:14783-8.

Bosutinib, 4-(2,4-dichloro-5-methoxy-phenylamino)-6-methoxy-7-[3-(4-methyl-piperizin-1-yl)-propoxy]-quinoline-3-carbonitrile), is a novel quinoline compound with in vivo antitumor activity against cancer. Elevated Src-family kinase activity has been reported in a variety of tumor types, and is correlated with aggressiveness. In breast cancer, Src is activated when either HER2 or EGFR are overexpressed, and breast tumor lines exhibit increased sensitivity to Src inhibition under these conditions. Src activity is required for cytoplasmic signaling by estrogen receptor, while constitutive activation of this Src-dependent pathway can lead to estrogen-independence, as described by Finn R S, “Targeting Src in breast cancer,” in Ann Oncol; 19: 1379-1386, 2008. Studies indicate that Src up-regulation occurs in approximately 60% of breast cancer tumors. Pharmacological inhibition of Src suppresses growth and invasive behavior of breast tumor cells in vitro, as described by Jallal H, Valentino M L, Chen G, Boschelli F, Ali S, Rabbani S A, “A Src/Abl kinase inhibitor, SKI-606, blocks breast cancer invasion, growth, and metastasis in vitro and in vivo,” in Cancer Research 2007; 67:1580-8 and Vultur A, Buettner R, Kowolik C, et al, “SKI-606 (bosutinib), a novel Src kinase inhibitor, suppresses migration and invasion of human breast cancer cells,” in Mol Cancer Ther 2008; 7:1185-94.

Aromatase is an enzyme that converts androgens to estrone. Estrone can subsequently be converted to estradiol, which has been linked to increased growth or proliferation of estrogen receptor positive carcinoma. There are two types of aromatase inhibitors; steroidal (type I inhibitors) and non-steroidal inhibitors (type II inhibitors). For example, exemestane, 10,13-dimethyl-6-methylidene-7,8,9,10,11,12,13,14,15,16-deca-hydrocyclopenta[a]phen-anthrene-3,17-dione, is an steroidal aromatase inhibitor and exhibits antitumor activity when administered as a single agent to breast cancer subjects. Exemestane is used in the adjuvant treatment of hormonally-responsive, also called hormone-receptor-positive, estrogen-responsive, breast cancer in postmenopausal women. Tamoxifen is another example of a single agent aromatase inhibitor that is used for the treatment of both early and advanced ER+ (estrogen receptor positive) breast cancer in pre- and post-menopausal women. These treatments are effective and provide significant improvement in overall and disease-free survival, as described by Eisen, A., Trudeau, M., Shelley, W., Messersmith, H., and Pritchard, K. I., “Aromatase inhibitors in adjuvant therapy for hormone receptor positive breast cancer: a systematic review,” Cancer Treat Rev, 34: 157-174, 2008. Nonetheless, recurrences will develop in a significant number of patients undergoing surgery and subsequent hormone therapy, as described by Herold, C. I. and Blackwell, K. L., “The impact of adjuvant endocrine therapy on reducing the risk of distant metastases in hormone-responsive breast cancer,” Breast, 17 Suppl 1: S15-24, 2008 and Rugo, H. S., “The importance of distant metastases in hormone-sensitive breast cancer,” Breast, 17 Suppl 1: S3-8, 2008.

International patent Publication WO2007099335 describes certain Platelet-derived growth factor (PDGF) inhibitors, namely quinolin-4-yloxy-substituted 2-(2-pyrimidinyl)acetamide compounds in combination with other Src inhibitors. The reference also suggests combinations of certain PDGF inhibitors and aromatase inhibitors. No combination of an Src inhibitor and the aromatase inhibitors exemestane, tamoxifen or letrozole is disclosed.

### SUMMARY OF THE INVENTION

Accordingly, the present invention provides a pharmaceutical composition comprising a therapeutically effective amount of a Src kinase inhibitor, or a pharmaceutically acceptable salt thereof, a therapeutically effective amount of an aromatase inhibitor, or a pharmaceutically acceptable salt thereof, and a pharmaceutically acceptable carrier. Accordingly, the present invention provides a pharmaceutical composition comprising a therapeutically effective amount of 4-(2,4-dichloro-5-methoxy-phenylamino)-6-methoxy-7-[3-(4-methyl-piperizin-1-yl)-propoxy]-quinoline-3-carbonitrile) or a pharmaceutically acceptable salt thereof and a therapeutically effective amount of 4-[(4-cyanophenyl)-(1,2,4-triazol-1-yl)methyl]benzonitrile or a pharmaceutically acceptable salt thereof.

The present invention also provides a pharmaceutical composition comprising a therapeutically effective amount of a Src kinase inhibitor, or a pharmaceutically acceptable salt thereof, a therapeutically effective amount of an aromatase inhibitor, or a pharmaceutically acceptable salt thereof, and a pharmaceutically acceptable carrier, wherein the amounts of the Src kinase inhibitor and the aromatase inhibitor in the composition are such that the combined therapeutic effect of the Src inhibitor and the aromatase inhibitor is synergistic. The present invention also provides a pharmaceutical composition further comprising a therapeutically effective amount of 4-(2,4-dichloro-5-methoxy-phenylamino)-6-methoxy-7-[3-(4-methyl-piperizin-1-yl)-propoxy]-quinoline-3-carbonitrile) or a pharmaceutically acceptable salt thereof, a therapeutically effective amount of 4-[(4-cyanophenyl)-(1,2,4-triazol-1-yl)methyl]benzonitrile or a pharmaceutically acceptable salt thereof and a pharmaceutically acceptable carrier. The present invention also provides a pharmaceutical composition wherein the amounts of the Src kinase inhibitor and the aromatase inhibitor in the composition are such that the combined therapeutic effect is synergistic.

The present invention also provides a composition comprising a therapeutically effective amount of a Src kinase inhibitor selected from bosutinib, dasatinib, neratinib, PP1, PP2, AP23464 and PD166326 or a pharmaceutically acceptable salt thereof, an aromatase inhibitor selected from anastrozole, letrozole, vorazole, tamoxifen and exemestane or a pharmaceutically acceptable salt thereof, and a pharmaceutically acceptable carrier.

The invention also provides a pharmaceutical pack for treating a neoplasm in one individual mammal which comprises (a) at least one unit dose of an aromatase inhibitor a pharmaceutically acceptable salt thereof; and (b) at least one unit dose of an Src inhibitor or a pharmaceutically acceptable salt thereof. The invention also provides a pharmaceutical pack for treating a neoplasm in one individual mammal which comprises (a) at least one unit dose of 4-(2,4-dichloro-5-methoxy-phenylamino)-6-methoxy-7-[3-(4-methyl-piperizin-1-yl)-propoxy]-quinoline-3-carbonitrile) or a pharmaceutically acceptable salt thereof; and (b) at least one unit dose of 4-[(4-cyanophenyl)-(1,2,4-triazol-1-yl)methyl]benzonitrile or a pharmaceutically acceptable salt thereof.

The present invention provides a method of treating cancer in a mammal, including a human, comprising administering to a mammal in need of such treatment a therapeutically effective amount of a Src kinase inhibitor, or a pharmaceutically acceptable salt thereof, and a therapeutically effective amount of an aromatase inhibitor, or a pharmaceutically acceptable salt thereof, and a pharmaceutically acceptable carrier, wherein the cancer is selected from: breast, kidney, bladder, thyroid, mouth, larynx, esophagus, stomach, colon, ovary, lung, pancreas, skin, liver, prostate and brain cancer. The present invention provides a method of treating cancer in a mammal, including a human, comprising administering to a mammal in need of such treatment a therapeutically effective amount of 4-(2,4-dichloro-5-methoxy-phenylamino)-6-methoxy-7-[3-(4-methyl-piperizin-1-yl)-propoxy]-quinoline-3-carbonitrile) or a pharmaceutically acceptable salt thereof, and a therapeutically effective amount of 4-[(4-cyanophenyl)-(1,2,4-triazol-1-yl)methyl]benzonitrile or a pharmaceutically acceptable salt thereof, and a pharmaceutically acceptable carrier, wherein the cancer is selected from: breast, kidney, bladder, thyroid, mouth, larynx, esophagus, stomach, colon, ovary, lung, pancreas, skin, liver, prostate and brain cancer.

The present invention provides a method of treating cancer in a mammal, including a human, comprising administering to a mammal in need of such treatment a therapeutically effective amount of a Src kinase inhibitor, or a pharmaceutically acceptable salt thereof, and a therapeutically effective amount of an aromatase inhibitor, or a pharmaceutically acceptable salt thereof, and a pharmaceutically acceptable carrier, wherein the amounts of the Src kinase inhibitor and the aromatase inhibitor in the composition are such that the combined therapeutic effect of the Src inhibitor and exemestane is synergistic and wherein the cancer is selected from: breast, kidney, bladder, thyroid, mouth, larynx, esophagus, stomach, colon, ovary, lung, pancreas, skin, liver, prostate and brain cancer. The present invention provides a method of treating cancer in a mammal, including a human, comprising administering to a mammal in need of such treatment a therapeutically effective amount of 4-(2,4-dichloro-5-methoxy-phenylamino)-6-methoxy-7-[3-(4-methyl-piperizin-1-yl)-propoxy]-quinoline-3-carbonitrile) or a pharmaceutically acceptable salt thereof, and a therapeutically effective amount of 4-[(4-cyanophenyl)-(1,2,4-triazol-1-yl)methyl]benzonitrile or a pharmaceutically acceptable salt thereof, and a pharmaceutically acceptable carrier, wherein the amounts in the composition are such that the combined therapeutic effect is synergistic and wherein the cancer is selected from: breast, kidney, bladder, thyroid, mouth, larynx, esophagus, stomach, colon, ovary, lung, pancreas, skin, liver, prostate and brain cancer.

The present invention also provides a combination therapy that is useful for treatment of breast cancer comprising the pharmaceutical composition of the invention. The present invention also provides a combination therapy which is utilized for treatment of ER positive metastatic or locally advanced breast cancer comprising the pharmaceutical composition of the invention.

The present invention provides a method of treating mammalian diseases associated with a non-receptor tyrosine kinase by administering to a patient a therapeutically effective amount of a Src kinase inhibitor, or a pharmaceutically acceptable salt thereof, a therapeutically effective amount of an aromatase inhibitor, or a pharmaceutically acceptable salt thereof, and a pharmaceutically acceptable carrier. The present invention provides a method of treating mammalian diseases associated with a non-receptor tyrosine kinase by administering to a patient a therapeutically effective amount of 4-(2,4-dichloro-5-methoxy-phenylamino)-6-methoxy-7-[3-(4-methyl-piperizin-1-yl)-propoxy]-quinoline-3-carbonitrile) or a pharmaceutically acceptable salt thereof, a therapeutically effective amount of 4-[(4-cyanophenyl)-(1,2,4-triazol-1-yl)methyl]benzonitrile or a pharmaceutically acceptable salt thereof, and a pharmaceutically acceptable carrier.

The present invention provides a method of treating mammalian diseases associated with a non-receptor tyrosine kinase by administering, to a patient a therapeutically effective amount of a Src kinase inhibitor, or a pharmaceutically acceptable salt thereof, a therapeutically effective amount of an aromatase inhibitor, or a pharmaceutically acceptable salt thereof, and a pharmaceutically acceptable carrier, wherein the amounts of the Src kinase inhibitor and the aromatase inhibitor in the composition are such that the combined therapeutic effect of the Src inhibitor and the aromatase inhibitor is synergistic.

Also provided is a medicament for use in the treatment of cell proliferative disorders or in the treatment of disease states associated with angiogenesis and/or vascular permeability comprising a therapeutically effective amount of a Src kinase inhibitor, or a pharmaceutically acceptable salt thereof, and a therapeutically effective amount of an aromatase inhibitor, or a pharmaceutically acceptable salt thereof, and a pharmaceutically acceptable carrier. There is also provided a medicament for use in the treatment of cell proliferative disorders or in the treatment of disease states associated with angiogenesis and/or vascular permeability comprising a therapeutically effective amount of 4-(2,4-dichloro-5-methoxy-phenylamino)-6-methoxy-7-[3-(4-methyl-piperizin-1-yl)-propoxy]-quinoline-3-carbonitrile) or a pharmaceutically acceptable salt thereof, and a therapeutically effective amount of 4-[(4-cyanophenyl)-(1,2,4-triazol-1-yl)methyl]benzonitrile or a pharmaceutically acceptable salt thereof, and a pharmaceutically acceptable carrier.

### DETAILED DESCRIPTION OF THE INVENTION

### Definitions

As used herein, the term “individual”, “subject” or “patient,” used interchangeably, refers to any animal, including mammals, preferably mice, rats, other rodents, rabbits, dogs, cats, swine, cattle, sheep, horses, or primates, and most preferably humans.

The term “treating” or “treatment” refers to any indicia of success in amelioration of an injury, pathology, or condition, including any objective or subjective parameter such as abatement; remission; diminishing of symptoms or making the injury, pathology, or condition more tolerable to the patient; slowing the rate of degeneration or decline; making the final point of degeneration less debilitating; or improving a subject's physical or mental well-being. The treatment or amelioration of symptoms can be based on objective or subjective parameters; including the results of a physical examination, neurological examination, and/or psychiatric evaluation. “Treating” or “treatment of a non-receptor tyrosine kinase related disorder” includes preventing the onset of symptoms in a subject that may be predisposed to a non-receptor tyrosine kinase related disorder but does not yet experience or exhibit symptoms of the disorder (prophylactic treatment), inhibiting the symptoms of the disorder (slowing or arresting its development), providing relief from the symptoms or side-effects of the disorder (including palliative treatment), and/or relieving the symptoms of the disorder (causing regression). Accordingly, the term “treating” includes the administration of the compounds or agents of the present invention to a subject to prevent or delay, to alleviate, or to arrest or inhibit development of the symptoms or conditions associated with non-receptor tyrosine kinase related disorders, e.g., tumor growth associated with cancer. A skilled medical practitioner will know how to use standard methods to determine whether a patient is suffering from a disease associated with overexpression and/or activity of a non-receptor tyrosine kinase, e.g., by examining the patient and determining whether the patient is suffering from a disease known to be associated with overexpression of a non-receptor tyrosine kinase or activity or by assaying for non-receptor tyrosine kinase levels in blood plasma or tissue of the individual suspected of suffering from a non-receptor tyrosine kinase related disease and comparing kinase levels in the blood plasma or tissue of the individual suspected of suffering from a non-receptor tyrosine kinase related disease to kinase levels in the blood plasma or tissue of a healthy individual. Overexpression of non-receptor tyrosine kinase levels are indicative of disease. The invented pharmaceutical composition is effective in overexpressed ER+ levels, indicative of cancers, including locally advanced or metastatic ER+/PgR+/Her2-breast cancer. Accordingly, the present invention provides, inter alia, methods of administering a compound of the present invention to a subject and determining overexpression of non-receptor tyrosine kinase levels in the subject. The level of non-receptor tyrosine kinase in the subject can be determined before and/or after administration of the compound.

As used herein, the term “therapeutically effective amount” refers to the amount of active compound or pharmaceutical agent that elicits the biological or medicinal response in a tissue, system, animal, individual or human that is being sought by a researcher, veterinarian, medical doctor or other clinician, which includes one or more of the following: (1) preventing the disease; for example, preventing a disease, condition or disorder in an individual that may be predisposed to the disease, condition or disorder but does not yet experience or display the pathology or symptomatology of the disease; (2) inhibiting the disease; for example, inhibiting a disease, condition or disorder in an individual that is experiencing or displaying the pathology or symptomatology of the disease, condition or disorder (i.e., arresting or slowing further development of the pathology and/or symptomatology); and (3) ameliorating the disease; for example, ameliorating a disease, condition or disorder in an individual that is experiencing or displaying the pathology or symptomatology of the disease, condition or disorder (i.e., reversing the pathology and/or symptomatology).

As used herein, the term “pharmaceutically acceptable carrier” includes pharmaceutically acceptable excipients, diluents, fillers, disintegrants, lubricants and other agents that can function as a carrier. The term “pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous. Pharmaceutical compositions are prepared in accordance with acceptable pharmaceutical procedures, such as described in Remingtons Pharmaceutical Sciences, 17th edition, ed. Alfonoso R. Gennaro, Mack Publishing Company, Easton, Pa. (1985). Pharmaceutically acceptable carriers are those that are compatible with the other ingredients in the formulation and biologically acceptable.

As used herein, the term “combination” or “combined preparation” refers to a Src kinase inhibitor and an aromatase inhibitor either in admixture, i.e. in the same pharmaceutical composition, or as separate components, e.g. in separate pharmaceutical compositions, or in separate unit dosage forms within a pharmaceutical pack, for use together, i.e. as a “combined therapy” or “combined regimen”, in the treatment of the disease states as defined herein. Accordingly when used in therapy the combination or combined preparation can be administered simultaneously, separately or sequentially. When the combination is administered simultaneously this can either be as a single pharmaceutical composition comprising both a Src kinase inhibitor and an aromatase inhibitor, or as separate compositions each comprising a Src kinase inhibitor or an aromatase inhibitor. When such a combination is administered separately or sequentially, the Src kinase inhibitor may be administered to the patient before or after the aromatase inhibitor.

According to one embodiment, the present invention provides a pharmaceutical composition comprising a therapeutically effective amount of a Src kinase inhibitor, or a pharmaceutically acceptable salt thereof, a therapeutically effective amount of an aromatase inhibitor, or a pharmaceutically acceptable salt thereof, and a pharmaceutically acceptable carrier.

Src inhibitors usefully employed in accordance with the invention are certain 4-anilino-3-cyanoquinolines. Suitable examples are of Src inhibitors include, but are not limited to, 4-anilino-3-cyanoquinolines described in U.S. Pat. Nos. 6,002,008; 6,288,082; 6,297,258; 6,780,996; 7,297,795 and 7,399,865. Suitable examples of Src inhibitors include, but are not limited to bosutinib, 4-(2,4-dichloro-5-methoxy-phenylamino)-6-methoxy-7-[3-(4-methyl-piperizin-1-yl)-propoxy]-quinoline-3-carbonitrile), neratinib, (E)-N-{4-[3-chloro-4-(2-pyridinyl methoxy) anilino]-3-cyano-7-ethoxy-6-quinolinyl}-4-(dimethylamino)-2-butenamide, (E)-N-(4-{3-chloro-4-[(3-fluorobenzyl)oxy]anilino}-3-cyano-7-ethoxy-6-quinolinyl)-4-(dimethylamino)-2-butenamide, (2E)-N-(4-{[3-chloro-4-(1,3-thiazol-2-ylsulfanyl)phenyl]amino}-3-cyano-7-methoxy-6-quinolinyl)-4-(dimethylamino)-2-butenamide, (E)-N-(4-{3-chloro-4-[(4,6-di-methyl-2-pyrimidinyl)-sulfanyl]anilino}-3-cyano-7-ethoxy-6-quinolinyl)-4-(dimethylamino)-2-butenamide, (E)-N-{4-[3-chloro-4-(1,3-thiazol-2-ylsulfanyl)anilino]-3-cyano-7-methoxy-6-quinolinyl}-4-[(2-methoxy-ethyl)(methyl)amino]-2-butenamide, dasatinib, N-(2-chloro-6-methylphenyl)-2-[[6-[4-(2-hydroxyethyl)-1-piperazinyl]-2-methyl-4-pyrimidinyl]amino]-5-thiazole carboxamide monohydrate, 4-(4-Chloro-2-fluoroanilino)-7-[5-(4-morpholinylmethyl)-3-thienyl]-3-quinolinecarbonitrile, 4-(2,4-Dichloro-5-methoxy-anilino)-7-[(E)-2-(4-pyridinyl)ethenyl]-3-quinolinecarbonitrile, 4-(2,4-Dichloro-5-methoxyanilino)-7-[(E)-2-(2-pyridinyl)-ethenyl]-3-quinolinecarbonitrile, 4-(2,4-Dichloroanilino)-7-[(E)-2-(4-pyridinyl)ethenyl]-3-quinolinecarbonitrile, 4-(2,4-Dichloro-5-methoxyanilino)-7-[5-(1,3-dioxolan-2-yl)-2-furyl]-3-quinolinecarbonitrile, (2,4-Dichloro-5-methoxyanilino)-7-(5-formyl-2-furyl)-3-quinolinecarbonitrile, 7[5-(4-Morpholinylmethyl)-3-thienyl]-4-(4-phenoxyanilino)-3-quinolinecarbonitrile, 4-(4-Benzylanilino)-7-[5-(4-morpholinylmethyl)-3-thienyl]-3-quinolinecarbonitrile, 4-(2,4-Dichloroanilino)-7-{5-[2-(4-morpholinyl)ethyl]-2-thienyl}-3-quinolincarbonitrile, 4-(2,4-Dichloroanilino)-7-{5-[(4-ethyl-1-piperazinyl)methyl]-3-thienyl}-3-quinolinecarbonitrile, 4-(2,4-Dichloroanilino)-7-[5-(4-morpholinyl)1-pentynyl]-3-quinolinecarbonitrile, 4-(2,4-Dichloroanilino)-7-[(E/Z)-5-(4-morpholinyl)-1-pentenyl]-3-quinolinecarbonitrile, 4-(2,4-Dichloroanilino)-7-[5-(4-morpholinylmethyl)-2-furyl]-3-quinolinecarbonitrile, 4-(2,4-Dichloroarnilino)-7-(3-hydroxy-1-propynyl)-3-quinolinecarbonitrile, 4-(2,4-Dichloroanilino)-7-[3-(dimethylamino)-l1-propynyl]-3-quinolinecarbonitrile, 4-(2,4-Dichloroanilino)-7-[(E/Z)-6-(4-morpholinyl)-1-hexenyl]-3-quinolinecarbonitrile, 7-[4,5-Bis(4-morpholinylmethyl)-2-thienyl]4-(2,4-dichloranilino)3-quinolinecarbonitrile, 4-(2,4-Dichloroanilino)-7-[5-(2-pyridinyl)-2-thienyl]-3-quinolinecarbonitrile, 4-(2,4-Dichloro-5-methoxyanilino)-7-{5-[(4-ethyl-1-piperazinyl)methyl]-3-thienyl}-3-quinolinecarbonitrile, 7-[4,5-Bis(4-morpholinylmethyl)-2-thienyl]-4-(2,4-dichloro-5-methoxyanilino)-3-quinolinecarbonitrile, 4-(2,4-Dichloro-5-methoxyanilino)-7-{5-[(E)-3-(4-morpholinyl)-1-propenyl]-2-thienyl}-3-quinolinecarbonitrile, 4-(2,4-Dichloro-5-methoxyanilino)-7-{5-[4-(4-morpholinyl)butyl]-2-thienyl}3-quinolinecarbonitrile, 4-(2,4-Dichloro-5-methoxyanilino)-7-[5-(4-morpholinylmethyl)-2-thienyl]-3-quinolinecarbonitrile, 4-(2,4-Dichloro-5-methoxyanilino)-7-[5-(4-morpholinylmethyl)-3-thienyl]-3-quinolinecarbonitrile, 4-(2,4-Dichloroanilino)-7-[5-(4-morpholinylmethyl)-3-thienyl]-3-quinolinecarbonitrile, 4-(2,4-Dichloro-5-methoxyanilino)-7-[3-(4-morpholinylmethyl)phenyl]-3-quinolinecarbonitrile, 4-(2,4-Dichloro-5-methoxyanilino)-7-{4-[2-(4-morpholinyl)ethyl]phenyl}-3-quinolinecarbonitrile, 4-(2,4-Dichloro-5-methoxyanilino)-7-{3-[2-(4-morpholinyl)ethyl]phenyl}-3-quinolinecarbonitrile, 4-(2,4-Dichloro-5-methoxyanilino)-7-[4-(4-morpholinylmethyl)phenyl]-3-quinolinecarbonitrile, 4-(2,4-Dichloro-5-methoxyanilino)-7-{4-[(4-ethyl-1-piperazinyl)methyl]phenyl}-3-quinolinecarbonitrile, 4-(2,4-Dichloro-5-methoxyanilino)-7-{4-[(4-ethyl-1-piperazinyl)methyl]phenyl}-6-methoxy-3-quinolinecarbonitrile, 4-(2,4-Dichloro-5-methoxyanilino)-7-{4-[2-(4-ethyl-1-piperazinyl)ethyl]phenyl}-3-quinolinecarbonitrile, 4-{3-Chloro-4-[(1-methyl-1H-imidazol-2-yl)sulfanyl]anilino}-7-[5-(4-morpholinylmethyl)-3-thienyl]-3-quinolinecarbonitrile, 7-[3,4-Bis(4-morpholinylmethyl)phenyl]-4-(2,4-dichloro-5-methoxyanilino)-3-quinolinecarbonitrile, 7-[3,4-Bis(4-morpholinylmethyl)-phenyl]-4-{3-chloro-4-[(1-methyl-1H-imidazol-2-yl)sulfanyl]anilino}-3-quinoline-carbonitrile, 4-(2,4-Dichloro-5-methoxyanilino)-6-methoxy-7-[5-(4-morpholinylmethyl)-3-thienyl]-3-quinolinecarbonitrile, 4-(2,4-Dichloro-5-methoxyanilino)-6-methoxy-7-[4-(4-morpholinylmethyl)phenyl]-3-quinolinecarbonitrile, 4-(2,4-Dichloro-5-methoxyanilino)-6-methoxy-7-{3-[2-(4-morpholinyl)ethyl]phenyl}-3-quinolinecarbonitrile, 4-(2,4-Dichloro-5-methoxyanilino)-6-methoxy-7-[3-(4-morpholinylmethyl]phenyl}-3-quinolinecarbonitrile, 4-(2,4-Dichloro-5-methoxyanilino)-6-methoxy-7-{4-[2-(4-morpholinyl)ethyl]phenyl}-3-quinolinecarbonitrile, 4-(2,4-Dichloro-5-methoxyanilino)-7-{5-[(4-ethyl-1-piperazinyl)methyl]-2-furyl}3-quinolinecarbonitrile, 4-(2,4-Dichloro-5-methoxyanilino)-7-[5-(1,3-dioxolan-2-yl)-3-thienyl]-3-quinolinecarbonitrile, 4-(2,4-Dichloro-5-methoxyanilino)-7-(5-formyl-3-thienyl)-3-quinoline-carbonitrile, 4-(2,4-Dichloroanilino)-7-(5-formyl-3-thienyl)-3-quinolinecarbonitrile, 4-(2,4-Dichloro-5-methoxyanilino)-6-(5-formyl-3-thienyl)-3-quinolinecarbonitrile, 4-(2,4-Dichloro-5-methoxyanilino)-7-{5-[(4-methyl-1-piperazinyl)methyl]-3-thienyl}-3-quinolinecarbonitrile, (2R)-1-({5-[3-Cyano-4-(2,4-dichloro-5-methoxyanilino)-7-quinolinyl]-2-furyl}methyl)-2-pyrrolidinecarboxamide, 7-[5-(4-Morpholinylmethyl)-3-pyridinyl]-4-(4-phenoxyanilino)-3-quinolinecarbonitrile, 4-(2,4-Dichloro-5-methoxyanilino)-7-[5-(4-morpholinylmethyl)-3-pyridinyl]-3-quinolinecarbonitrile, 4-(2,4-Dichloro-5-methoxyanilino)-6-[5-(4-morpholinylmethyl)-3-thienyl]-3-quinoline-carbonitrile, 4-(2,4-Dichloro-5-methoxyanilino)-7-[5-(1,3-dioxolan-2-yl)-2-thienyl]-3-quinolinecarbonitrile, 4-(2,4-Dichloro-5-methoxyanilino)-7-(5-formyl-2-thienyl)-3-quinolinecarbonitrile, 4-(2,4-Dichloro-5-methoxyanilino)-7-[5-(4-morpholinylmethyl)-2-furyl]-3-quinolinecarbonitrile, 4-(2,4-Dichloro-5-methoxyanilino)-7-[(E)-2-(4-methoxyphenyl)ethenyl]-3-quinolinecarbonitrile, 4-(2,4-Dichloro-5-methoxyanilino)-7-{5-[(4-methyl-1-piperazinyl)methyl]-2-thienyl}-3-quinolinecarbonitrile, 7-[5-(4-Morpholinylmethyl)-2-pyridinyl]-4-(4-phenoxyanilino)-3-quinolinecarbonitrile, 4-(2,4-Dichloro-5-methoxyanilino)-7-[5-(4-morpholinylmethyl)-2-pyridinyl]-3-quinolinecarbo-nitrile, 4-(2,4-Dichloro-5-methoxyanilino)-7-[5-({[2-(phenylsulfonyl)ethyl]amino}-methyl)-2-furyl]-3-quinolinecarbonitrile, 4-(2,4-Dichloro-5-methoxyanilino)-6-methoxy-7-(1H-pyrrol-1-yl)-3-quinolinecarbonitrile, 4-(3-Bromoanilino)-6-(2-formyl-1H-pyrrol-1-yl)-3-quinolinecarbonitrile, 4-(3-Chloro-4-fluoro-phenylamino)-7-methoxy-6-(1H-pyrrol-1-yl)-3-quinolinecarbonitrile, 4-{3-Chloro-4-[(1-methyl-1H-imidazol-2-yl)sulfanyl]anilino}-7-(4-formylphenyl)-3-quinolinecarbonitrile, 4-{3-Chloro-4-[(1-methyl-1H-imidazol-2-yl)sulfanyl]anilino}-7-[4-(4-morpholinylmethyl)phenyl]-3-quinolinecarbonitrile, 4-(2,4-Dichloro-5-methoxyanilino)-7-{1-[2-(4-morpholinyl)ethyl]-1H-imidazol-5-yl}3-quinolinecarbonitrile, 4-(2,4-Dichloro-5-methoxyanilino)₇-[4-(4-morpholinylmethyl)-3-thienyl]3-quinolinecarbonitrile, 4-(2,4-Dichloro-5-methoxy-anilino)-7-[2-(4-morpholinylmethyl)-3-thienyl]-3-quinolinecarbonitrile, 4-(2,4-Dichloro-5-methoxyanilino)-7-[4-(4-morpholinyl)phenyl]-3-quinolinecarbonitrile, 4-(2,4-Dichloro-5-methoxyanilino)-7-[4-(4-morpholinylmethyl)-2-thienyl]-3-quinolinecarbo-nitrile, 4-(2,4-Dichloro-5-methoxyanilino)-7-(5-formyl-1-methyl-1H-pyrrol-2-yl)-3-quinolinecarbonitrile, 4-(2,4-Dichloro-5-methoxyanilino)-7-[1-methyl-5-(4-morpholinylmethyl)-1H-pyrrol-2-yl]-3-quinolinecarbonitrile, 4-(2,4-Dichloro-5-methoxyanilino)-7-{1-methyl-5-[(4-methyl-1-piperazinyl)methyl]-1H-pyrrol-2-yl}-3-quinolinecarbonitrile, 4-(2,4-Dichloro-5-methoxyanilino)-7-[1-methyl-5-({[2-(phenylsulfonyl)ethyl]amino}methyl)-1H-pyrrol-2-yl]-3-quinolinecarbonitrile, 4-(2,4-Dichloro-5-methoxyanilino)-7-[1-methyl-5-({[2-(methylsulfonyl)ethyl]-amino}methyl)-1H-pyrrol-2-yl]-3-quinolinecarbonitrile, 4-(2,4-Dichloro-5-methoxyanilino)-7-[5-({[2-(2-pyridinyl)ethyl]amino}methyl)-2-furyl]-3-quinolinecarbonitrile, 4-(2,4-Dichloro-5-methoxyanilino)-7-(5-{[4-(2-hydroxyethyl)-1-piperazinyl]methyl}-2-furyl)-3-quinoline-carbonitrile, 7-(5-{[Bis(2-hydroxyethyl)amino]methyl}-2-furyl)-4-(2,4-dichloro-5-methoxyanilino)-3-quinolinecarbonitrile, 4-(2,4-Dichloro-5-methoxyanilino)-7-[5-({[2-(methylsulfonyl)ethyl]amino}methyl)-2-furyl]-3-quinolinecarbonitrile, 4-(2,4-Dichloro-5-methoxyanilino)-7-[5-(1-piperidinyl)methyl)-2-thienyl]-3-quinolinecarbonitrile, 4-{2-Chloro-4-fluoro-5-methoxyanilino)-7-[5-(4-morpholinylmethyl)-3-thienyl]-3-quinoline-carbonitrile, 4-{2-Chloro-5-methoxy-4-methylanilino)-7-[5-(4-morpholinylmethyl)-3-thienyl]-3-quinolinecarbonitrile, 4-(2,4-Dichloro-5-methoxyanilino)-7-[6-(4-morpholinylmethyl)-3-pyridinyl]-3-quninolinecarbonitrile, 7-[4,5-Bis(4-morpholinyl-methyl)-2-thienyl]-4-(2,4-dichloro-5-methoxyanilino)-6-methoxy-3-quinolinecarbo-nitrile, 4-(2,4-Dichloro-5-methoxyanilino)-7-(4-formylphenyl)-3-quinolinecarbonitrile, (2R)-1-{4-[3-Cyano-4-(2,4-dichloro-5-methoxyanilino)-7-quinolinyl]benzyl}-2-pyrrolidinecarboxamide, 4-(2,4-Dichloro-5-methoxyanilino)-7-[4-({[2-(phenylsulfonyl)-ethyl]amino}methyl)-phenyl]-3-quinolinecarbonitrile, 4-(2,4-Dichloro-5-methoxy-anilino)-7-{4-[(dimethylamino)methyl]phenyl}-3-quinolinecarbonitrile, 4-(2,4-Dichloro-5-methoxyanilino)-7-{4-[(diethylamino)methyl]phenyl}-3-quinolinecarbonitrile, 4-(2,4-Dichloro-5-methoxyanilino)-7-[4-({[2-(methylsulfonyl)ethyl]amino}methyl)-phenyl]-3-quinolinecarbonitrile, 4-(2,4-Dichloro-5-methoxyanilino)-7-{5-[(4-hydroxy-1-piperidinyl)methyl]-2-thienyl}-3-quinolinecarbonitrile, 4-(2,4-Dichloro-5-methoxy-anilino)-7-[2-(4-methoxyphenyl)ethynyl]-3-quinolinecarbonitrile, 4-(2,4-Dichloro-5-methoxyanilino)-7-[2-(2-pyridinyl)ethynyl]-3-quinolinecarbonitrile, 4-(2,4-Dichloro-5-methoxyanilino)-7-pyrrol-1-yl-3-quinolinecarbonitrile, 4-(2,4-Dichloro-5-methoxy-anilino)-7-{(2-[(dimethylamino)methyl]-1H-pyrrol-1-yl}-3-quinolinecarbonitrile, 7-[5-(1,3-Dioxolan-2-yl)-3-thienyl]-4-[3-methyl-4-(2-pyridinylmethoxy)anilino]-3-quinoline-carbonitrile, 4-[3-Methyl-4-(2-pyridinylmethoxy)anilino]-7-[5-(4-morpholinylmethyl)-3-thien yl]-3-quinolinecarbonitrile, 4-[(2,4-Dichloro-5-methoxyanilino]-7-(2-formyl-1-methyl-1H-imidazol-5-yl)-quinoline-3-carbonitrile, 4-({3-Chloro-4-[(1-methyl-1H-imidazol-2-yl)sulfanyl]phenyl}amino)-7-[4-(1-piperazinylmethyl)phenyl]-3-quinolinecarbonitrile, 4-({3-Chloro-4-[(1-methyl-1H-imidazol-2-yl)sulfanyl]phenyl}-amino)-7-}amino-7-{4-[(4-isopropyl-1-piperazinyl)methyl]phenyl}-3-quinolinecarbo-nitrile, (E)-3-[3-Cyano-4-(2,4-dichloro-5-methoxyanilino)-7-quinolinyl]-2-propenoic acid, (1-{4-[3-Cyano-4-(2,4-dichloro-5-methoxyanilino)-7-quinolinyl]benzyl}-4-piper idinyl)acetic acid, 4-(2,4-Dichloro-5-methoxyanilino)-7-[4-(hydroxymethyl)phenyl]-3-quinolinecarbonitrile, 7-[4-(Chloromethyl)phenyl]-4-(2,4-dichloro-5-methoxyanilino)-3-quinolinecarbonitrile, 4-[(2,4-Dichloro-5-methoxyphenyl)amino]-7-[4-(1H-1,2,3-triazol-1-ylmethyl)phenyl]-3-quinolinecarbonitrile, 4-(2,4-Dichloro-5-methoxyanilino)-7-(1H-pyrrol-2-yl)-3-quinolinecarbonitrile, 4-[(2,4-Dichloro-5-methoxyanilino]-7-[4-(1H-tetraazol-5-yl)phenyl]-3-quinolinecarbonitrile, 4-({3-Chloro-4-[(1-methyl-1H-imidazol-2-yl)sulfanyl]phenyl}amino)-7-(5-{[(2-hydroxyethyl)(methyl)amino]methyl}-2-pyridinyl)-3-quinolinecarbonitrile, Methyl 1-{[6-(4-{3-chloro-4-[(1-methyl-1H-imidazol-2-yl)sulfanyl]anilino}-3-cyano-7-quinolinyl)-3-pyridinyl]methyl}-4-piperidinecarboxylate, 4-({3-Chloro-4-[(1-methyl-1H-imidazol-2-yl)sulfanyl]phenyl}amino)-7-[5-(4-ethyl-1-piperazinyl)-2-pyridinyl]-3-quinolinecarbonitrile, 4-({3-Chloro-4-[(1-methyl-1H-imidazol-2-yl)sulfanyl]phenyl}amino)-7-[6-(4-morpholinylmethyl)-3-pyridinyl]-3-quinolinecarbo-nitrile, 4-({3-Chloro-4-[(1-methyl-1H-imidazol-2-yl)sulfanyl]phenyl}amino)-7-[6-(4-(thiomorpholinyl)-3-pyridinyl]-3-quinolinecarbonitrile, 4-(2,4-Dichloro-5-methoxyanilino)-7-[3-(morpholin-4-ylmethyl)-pyridin-2-yl]-3-quinolinecarbonitrile, 4-(2,4-Dichloro-5-methoxyanilino)-7-(3-formyl)-3-quinolinecarbonitrile, 4-(2,4-Dichloro-5-methoxy-anilino)-7-{3-[(4-methyl-1-piperazinyl)methyl]phenyl}-3-quinolinecarbonitrile, 4-(2,4-Dichloro-5-methoxyanilino)-7-(2-(4-formylphenyl-3-quinolinecarbonitrile, 4-(2,4-Dichloro-5-methoxyanilino)-7-[2-(4-morpholinylmethyl)phenyl]-3-quinolinecarbonitrile, 4-(2,4-Dichloro-5-methoxyanilino)-7-(1-naphthyl)-3-quinolinecarbonitrile, 4-(2,4-Dichloro-5-methoxyanilino)-7-(2-naphthyl)-3-quinolinecarbonitrile, N-{3-[3-Cyano-4-(2,4-dichloro-5-methoxyanilino)-7-quinolinyl]phenyl}acetamide, 7-(1-Benzothien-2-yl)-4-2,4-dichloro-5-methoxyanilino)-3-quinolinecarbonitrile, 7-(1-Benzothien-2-yl)-4-2,4-dichloro-5-methoxyanilino)-3-quinolinecarbonitrile, 4-[3-Cyano-4-(2,4-dichloro-5-methoxyanilino)-7-quinolinyl]benzoic acid, 4-(2,4-Dichloro-5-methoxyanilino)-7-(3-nitrophenyl)-3-quinolinecarbonitrile, 4-{3-Chloro-4-[(1-methyl-1H-imidazol-2-yl)sulfanyl)]anilino}-6-methoxy-7-[4-(4-morpholinylmethyl)phenyl]-3-quinolinecarbo-nitrile, 7-[3,4-Bis(4-morpholinylmethyl)phenyl]-4-{3-chloro-4-[(1-methyl-1H-imidazol-2-yl)sulfanyl]anilino}-6-methoxy-3-quinolinecarbonitrile, 4-{3-Chloro-4-[(1-methyl-1H-imidazol-2-yl)sulfanyl]anilino}-6-methoxy-7-[5-(4-morpholinylmethyl)-3-thienyl]-3-quinolinecarbonitrile, 4-{3-Chloro-4-[(1-methyl-1H-imidazol-2-yl)sulfanyl]anilino}-6-methoxy-7-{5-[(4-methyl-1-piperazinyl)methyl]-3-thienyl}-3-quinolinecarbonitrile, 4-{3-Chloro-4-[(1-methyl-1H-imidazol-2-yl)sulfanyl]anilino}-6-methoxy-7-(4-methoxyphenyl)-3-quinolinecarbonitrile, 4-[(2,4-Dichloro-5-methoxyphenyl)amino]-7-[4-(4-morpholinyl)phenyl]-3-quinolinecarbonitrile, 4-({3-Chloro-4-[(1-methyl-1H-imidazol-2-yl)sulfanyl]phenyl}amino)-7-[4-(4-morpholinylcarbonyl)phenyl]-3-quinolinecarbo-nitrile, 4-(2,4-Dichloro-5-methoxyanilino)-7-{4-[(2-methoxy)ethoxy]phenyl}-3-quinoline carbonitrile, 4-(2-Chloro-5-methoxyanilino)-7-[5-(4-morpholinylmethyl)-3-thienyl]-3-quinolinecarbonitrile, 4-[4-(Benzyloxy)-3-chloroanilino]-7-[3,4-bis(4-morpholinyl-methyl)phenyl]-3-quinolinecarbonitrile, 7-[3,4-Bis(4-morpholinylmethyl)phenyl]-4-(2-chloro-5-methoxy-4-methylanilino)-3-quinolinecarbonitrile, 4-{3-Chloro-4-[(1-methyl-1H-imidazol-2-yl)sulfanyl]anilino}-7-{4-[(4-hydroxy-1-piperidinyl)methyl]phenyl}-3-quinolinecarbonitrile, 4-{3-Chloro-4-[(1-methyl-1H-imidazol-2-yl)sulfanyl]anilino}-7-{4-[(4-methyl-1-piperazinyl)methyl]phenyl}-3-quinolinecarbonitrile, 4-{3-Chloro-4-[(1-methyl-1H-imidazol-2-yl)sulfanyl]anilino}-7-[4-(1-piperidinyl)methyl)phenyl]-3-quinolinecarbonitrile, tert-Butyl 4-{4-[4-({3-chloro-4-[(1-methyl-1H-imidazol-2-yl)sulfanyl]phenyl}amino)-3-cyano-7-quinolinyl]benzyl}-1-piperazinecarboxylate, 4-({3-Chloro-4-[(1-methyl-1H-imidazol-2-yl)sulfanyl]phenyl}amino)-7-{4-[(4-morpholinyl)methyl]phenyl}-3-quinolinecarbonitrile, 4(2,4-Dichloro-5-methoxyanilino)-7-[(E)-2-phenylethenyl]-3-quinolinecarbonitrile, 4-(2,4-Dichloro-5-methoxyanilino)-7-(2-phenylethynyl)-3-quinolinecarbonitrile, 4-(2,4-Dichloro-5-methoxyanilino)-7-[2-(4-methylphenyl)ethynyl]-3-quinolinecarbonitrile, tert-Butyl(E)-3-[3-cyano-4-(2,4-dichloro-5-methoxyanilino)-7-quinolinyl]-2-propenoate, 4-(2,4-Dichloro-5-methoxy-anilino)-7-(3-hydroxy-1-propynyl)-3-quinolinecarbonitrile, Ethyl (1-{4-[3-Cyano-4-(2,4-dichloro-5-methoxyanilino)-7-quinolinyl]benzyl}-4-piperidinyl)acetate, Ethyl 1-{4-[3-cyano-4-(2,4-dichloro-5-methoxyanilino)-7-quinolinyl]benzyl}-2-piperidinecarboxylate, 4-(2,4-Dichloro-5-methoxyanilino)-7-[3-(4-morpholinyl)-1-propynyl)-3-quinoline-carbonitrile, 1-{4-[3-Cyano-4-(2,4-dichloro-5-methoxyanilino)-7-quinolinyl]benzyl}-2-piperidinecarboxylic acid, Ethyl 1-(4-{3-cyano-4-[(2,4-dichloro-5-methoxyphenyl)amino]-7-quinolinyl}benzyl)-3-piperidinecarboxylate, 1-(4-{3-Cyano-4-[(2,4-dichloro-5-methoxyphenyl)amino]-7-quinolinyl}benzyl)-3-piperidinecarboxylic acid, 4-[(2,4-Dichloro-5-methoxyphenyl)amino]-7-{4-[(1,1-dioxido-4-thiomorpholinyl)methyl]phenyl}-3-quinolinecarbonitrile, 4-[(2,4-Dichloro-5-methoxyphenyl)amino]-7-{4-[(1-oxido-4-thiomorpholinyl)methyl]phenyl}-3-quinolinecarbonitrile, 7-(3-Chloro-1-propynyl)-4-[(2,4-dichloro-5-methoxyphenyl)amino]-3-quinolinecarbonitrile, 4-[(2,4-Dichloro-5-methoxyphenyl)amino]-7-[4-(4-thiomorpholinylmethyl)phenyl]-3-quinoline-carbonitrile, 4-{3-Chloro-4-[(1-methyl-1H-imidazol-2-yl)sulfanyl]anilino}-7-[5-(4-morpholinylmethyl)-2-furyl]-3-quinolinecarbonitrile, 4-{3-Chloro-4-[(1-methyl-1H-imidazol-2-yl)sulfanyl]anilino}-7-[5-(1-piperidinyl)methyl)-2-furyl]-3-quinolinecarbonitrile, 4-{3-Chloro-4-[(1-methyl-1H-imidazol-2-yl)sulfanyl]anilino}-7-{5-[(4-ethyl-1-piperazinyl)methyl]-2-furyl}-3-quinolinecarbonitrile, 4-{3-Chloro-4-[(1-methyl-1H-imidazol-2-yl)sulfanyl]anilino}-7-(5-{[4-(2-hydroxyethyl)-1-piperazinyl]methyl}-2-furyl)-3-quinolinecarbonitrile, 4-({3-Chloro-4-[(1-methyl-1H-imidazol-2-yl)sulfanyl]phenyl}-amino)-7-(5-formyl-2-furyl)-3-quinolinecarbonitrile, 4-{3-Chloro-4-[(1-methyl-1H-imidazol-2-yl)sulfanyl]anilino}-7-{5-[(4-hydroxy-1-piperidinyl)methyl]-2-thienyl}-3-quinolinecarbonitrile, 4-{3-Chloro-4-[(1-methyl-1H-imidazol-2-yl)sulfanyl]anilino}-7-[5-(1-piperidinyl)methyl)-2-thienyl]-3-quinolinecarbonitrile, 4-{3-Chloro-4-[(1-methyl-1H-imidazol-2-yl)sulfanyl]anilino}-7-(5-{[4-(2-hydroxyethyl)-1-piperazinyl]methyl}-2-thienyl)-3-quinolinecarbonitrile, 4-(2,4-Dichloro-5-methoxyanilino)-7-[5-(hydroxyl-methyl)-1-methyl-1H-pyrrol-2-yl]-3-quinolinecarbonitrile, 4-(2,4-Dichloro-5-methoxy-anilino)-7-(3-formyl-2-thienyl)-3-quinolinecarbonitrile, tert-Butyl 2-[3-cyano-4-(2,4-dichloro-5-methoxyanilino)-7-quinolinyl]-1H-pyrrole-1-carboxylate, 7-[1,1′-Biphenyl]-4-yl-4-(2,4-dichloro-5-methoxyanilino)-3-quinolinecarbonitrile, 4-(2,4-Dichloro-5-methoxyanilino)-6-methoxy-7-[3-(4-morpholinyl)-1-propynyl]-3-quinolinecarbonitrile, 4-(4-Chloro-5-methoxy-2-methylanilino)-7-[5-(4-morpholinylmethyl)-3-thienyl]-3-quinolinecarbonitrile, 7-[4,5-Bis(4-morpholinylmethyl)-2-thienyl]-4-(4-phenoxyanilino)-3-quinolinecarbonitrile, 7-[4,5-Bis(4-morpholinylmethyl)-2-thienyl]-4-{3-chloro-4-[(1-methyl-1H-imidazol-2-yl)sulfanyl]anilino}-3-quinolinecarbonitrile, 4-{3-Chloro-4-[(1-methyl-1H-imidazol-2-yl)sulfanyl]anilino}-7-(5-formyl-2-pyridinyl)-3-quinolinecarbo-nitrile, 4-{3-Chloro-4-[(1-methyl-1H-imidazol-2-yl)sulfanyl]anilino}-7-{5-[(4-ethyl-1-piperazinyl)methyl]-2-pyridinyl}-3-quinolinecarbonitrile, 4-{3-Chloro-4-[(1-methyl-1H-imidazol-2-yl)sulfanyl]anilino}-7-{5-[(4-hydroxy-1-piperidinyl)methyl]-2-pyridinyl}-3-quinolinecarbonitrile, 4-{3-Chloro-4-[(1-methyl-1H-imidazol-2-yl)sulfanyl]anilino}-7-(5-{[4-(1-pyrrolidinyl)-1-piperidinyl]methyl}-2-pyridinyl)-3-quinolinecarbonitrile, 7-(3-Aminophenyl)-4-[(2,4-dichloro-5-methoxyphenyl)amino]-3-quinolinecarbonitrile, 1-{[6-(4-{3-chloro-4-[(1-methyl-1H-imidazol-2-yl)sulfanyl]anilino}-3-cyano-7-quinolinyl)-3-pyridinyl]methyl}-4-piperidinecarboxylic acid, 1-{6-[3-Cyano-4-(2,4-dichloro-5-methoxyphenylamino)-quinolin-7-yl]-pyridin-3-ylmethyl}-piperidine-4-carboxylic acid methyl ester, 1-{6-[3-Cyano-4-(2,4-dichloro-5-methoxyphenylamino)-quinolin-7-yl]-pyridin-3-ylmethyl}-piperidine-4-carboxylic acid, 4-({3-Chloro-4-[(1-methyl-1H-imidazol-2-yl)sulfanyl]phenyl}amino)-7-(5-chloro-2-pyridinyl)-3-quinolinecarbonitrile, 4-[(2,4-Dichloro-5-methoxyphenyl)amino]-7-[5-(4-ethyl-1-piperazinyl)-2-pyridinyl]-3-quinolinecarbonitrile, 4-({3-Chloro-4-[(1-methyl-1H-imidazol-2-yl)sulfanyl]phenyl}-amino)-7-[5-(1-pyridinyl]-3-quinolinecarbonitrile, 4-({3-Chloro-4-[(1-methyl-1H-imidazol-2-yl)sulfanyl]phenyl}amino)-6-[5-(4-ethyl-1-piperazinyl)-2-pyridinyl]-3-quinolinecarbonitrile, 4-({3-Chloro-4-[(1-methyl-1H-imidazol-2-yl)sulfanyl]phenyl}-amino)-6-[5-(4-morpholinylmethyl)-2-pyridinyl]-3-quinolinecarbonitrile, 4-({3-Chloro-4-[(1-methyl-1H-imidazol-2-yl)sulfanyl]phenyl}amino)-6-{5-[(4-methyl-1-piperazinyl)-methyl]-2-pyridinyl}-3-quinolinecarbonitrile, 4-({3-Chloro-4-[(1-methyl-1H-imidazol-2-yl)sulfanyl]phenyl}amino)-6-[6-(4-morpholinyl)-3-pyridinyl]-3-quinolinecarbonitrile, 4-(2,4-Dichloro-5-methoxyanilino)-7-[6-(4-morpholinyl)-3-pyridinyl]-3-quinolinecarbo-nitrile, 4-(2,4-Dichloro-5-methoxyanilino)-7-[2-(4-morpholinyl)-5-pyridinyl]-3-quinoline-carbonitrile, 4-{3-Chloro-4-[(1-methyl-1H-imidazol-2-yl)sulfanyl]anilino}-7-[5-(4-morpholinylmethyl)-2-pyridinyl]-3-quinolinecarbonitrile, 4-(2,4-Dichloro-5-methoxy-anilino)-7-{5-[(4-methyl-1-piperazinyl)methyl]-2-pyridinyl}-3-quinolinecarbonitrile, 4-{3-Chloro-4-[(1-methyl-1H-imidazol-2-yl)sulfanyl]anilino}-7-{5-[(4-methyl-1-piperazinyl)-methyl]-2-pyridinyl}-3-quinolinecarbonitrile, 4-{3-Chloro-4-[(1-methyl-1H-imidazol-2-yl)sulfanyl]anilino}-7-[6-(4-morpholinyl)-3-pyridinyl]-3-quinolinecarbonitrile, 4-{3-Chloro-4-[(1-methyl-1H-imidazol-2-yl)sulfanyl]anilino}-7-[2-(4-morpholinyl)-5-pyrimidinyl]-3-quinolinecarbonitrile, 4-(2,4-Dichloro-5-methoxyanilino)-7-{5-[(4-hydroxy-1-piperidinyl)methyl]-3-thienyl}-3-quinolinecarbonitrile, 4-(2,4-Dichloro-5-methoxyanilino)-7-{6-[4-(4-morpholinylmethyl)phenoxy]-3-pyridinyl)-3-quinoline-carbonitrile, 4-(2,4-Dichloro-5-methoxyanilino)-7-(4-methoxyphenyl)-3-quinoline-carbonitrile, 4-{3-Chloro-4-[(1-methyl-1H-imidazol-2-yl)sulfanyl]anilino}-7-[6-(4-ethyl-1-piperazinyl)-3-pyridinyl]-3-quinolinecarbonitrile, 4-{3-Chloro-4-[(1-methyl-1H-imidazol-2-yl)sulfanyl]anilino}-7-[6-(4-methyl-1-piperazinyl)-3-pyridinyl]-3-quinoline-carbonitrile, 4-(2,4-Dichloro-5-methoxyanilino)-7-[6-(4-ethyl-1-piperazinyl)-3-pyridinyl]-3-quinolinecarbonitrile, 4-(2,4-Dichloro-5-methoxyanilino)-7-[6-(4-methyl-1-piperazinyl)-3-pyridinyl]-3-quinolinecarbonitrile, 4-{3-Chloro-4-[(1-methyl-1H-imidazol-2-yl)sulfanyl]anilino}-7-[6-(4-morpholinylmethyl)-2-pyridinyl]-3-quinolinecarbonitrile, 4-(2,4-Dichloro-5-methoxyanilino)-7-[6-(4-morpholinylmethyl)-2-pyridinyl]-3-quinoline-carbonitrile, 4-{3-Chloro-4-[(1-methyl-1H-imidazol-2-yl)sulfanyl]anilino}-7-{6-[(4-ethyl-1-piperazinyl)methyl]-2-pyridinyl}-3-quinolinecarbonitrile, 4-{3-Chloro-4-[(1-methyl-1H-imidazol-2-yl)sulfanyl]anilino}-7-{6-[(4-methyl-1-piperazinyl)methyl]-2-pyridinyl}-3-quinolinecarbonitrile, 4-({3Chloro-4-[(1-methyl-1H-imidazol-2-yl)sulfanyl]phenylamino)-7-[4-(4-morpholinylmethyl)-2-pyridinyl]-3-quinolinecarbonitrile, 4-[(2,4-Dichloro-5-methoxyphenyl)amino]-7-[4-(4-morpholinylmethyl)-2-pyridinyl]-3-quinolinecarbonitrile, 4-({3-Chloro-4-[(1-methyl-1H-imidazol-2-yl)sulfanyl]phenyl}amino)-7-{4-[(4-ethyl-1-piperazinyl)methyl]-2-pyridinyl}-3-quinolinecarbonitrile, 4-({3-Chloro-4-[(1-methyl-1H-imidazol-2-yl)sulfanyl]phenyl}amino)-7-{4-[(4-methyl-1-piperazinyl)methyl]-2-pyridinyl}-3-quinolinecarbonitrile, 4-({3-Chloro-4-[(1-methyl-1H-imidazol-2-yl)sulfanyl]-phenyl}amino)-7-[3-(4-morpholinylmethyl)-2-pyridinyl]-3-quinolinecarbonitrile, 4-({3-Chloro-4-[(1-methyl-1H-imidazol-2-yl)sulfanyl]phenyl}amino)-7-{3-[(4-ethyl-1-piperazinyl)methyl]-2-pyridinyl}-3-quinol inecarbonitrile, 4-({3-Chloro-4-[(1-methyl-1H-imidazol-2-yl)sulfanyl]phenyl}amino)-7-{3-[(4-methyl-1-piperazinyl)methyl]-2-pyridinyl}-3-quinolinecarbonitrile, 4-({3-Chloro-4-[(1-methyl-1H-imidazol-2-yl)sulfanyl]-phenyl}amino)-7-{6-[4-(1-pyrrolidinyl)-1-piperidinyl]-3-pyridinyl}-3-quinolinecarbonitrile, 4-({3-Chloro-4-[(1-methyl-1H-imidazol-2-yl)sulfanyl]phenyl}amino)-7-[6-(1-piperidinyl)-3-pyridinyl]-3-quinolinecarbonitrile, 4-({3-Chloro-4-[(1-methyl-1H-imidazol-2-yl)sulfanyl]-phenyl}amino)-7-{6-[(2-methoxyethyl)(methyl)amino]-3-pyridinyl}-3-quinol inecarbonitrile, Ethyl 1-{5-[4-({3-chloro-4-[(1-methyl-1H-imidazol-2-yl)sulfanyl]phenyl}amino)-3-cyano-7-quinolinyl]-2-pyridinyl}-4-piperidinecarboxylate, 4-({3-Chloro-4-[(1-methyl-1H-imidazol-2-yl)sulfanyl]phenyl}amino)-7-[6-(4-hydroxy-1-piperidinyl)-3-pyridinyl]-3-quinolinecarbonitrile, 4-({3-Chloro-4-[(1-methyl-1H-imidazol-2-yl)sulfanyl]phenyl}amino)-7-{6-[4-(2-hydroxyethyl)-1-piperazinyl]-3-pyridinyl}-3-quinolinecarbonitrile, 4-({3-Chloro-4-[(1-methyl-1H-imidazol-2-yl)sulfanyl]phenyl}-amino)-7-{6-[(2-hydroxyethyl)(methyl)amino]-3-pyridinyl}-3-quinol inecarbonitrile, 4-({3-chloro-4-[(1-methyl-1H-imidazol-2-yl)sulfanyl]phenyl}amino)-7-(5-{[4-(2-hydroxy-ethyl)-1-piperazinyl]-methyl}-2-pyridinyl)-3-quinolinecarbonitrile, 4-(2,4-Dichloro-5-methoxyanilino)-7-{4-[(4-methyl-1-piperazinyl)methyl]phenyl}-3-quinolinecarbonitrile, 4-({3-Chloro-4-[(1-methyl-1H-imidazol-2-yl)sulfanyl]phenyl}amino)-7-(5-thiomorpholinylmethyl)-2-pyridinyl]-3-quinolinecarbonitrile, 4-({3-Chloro-4-[(1-methyl-1H-imidazol-2-yl)sulfanyl]phenyl}amino)-7-{6-[(4-ethyl-1-piperazinyl)methyl]-3-pyridinyl}-3-quinolinecarbonitrile, 4-({3-Chloro-4-[(1-methyl-1H-imidazol-2-yl)sulfanyl]phenyl}-amino)-7-{6-[(4-methyl-1-piperazinyl)methyl]-3-pyridinyl}-3-quinolinecarbonitrile, 4-({3-Chloro-4-[(1-methyl-1H-imidazol-2-yl)sulfanyl]phenyl}amino)-7-(6-formyl-3-pyridinyl)-3-quinolinecarbonitrile, 4-({3-Chloro-4-[(1-methyl-1H-imidazol-2-yl)sulfanyl]phenyl}amino)-7-{6-[(4-hydroxy-1-piperidinyl)methyl]-3-pyridinyl}-3-quinolinecarbonitrile, 4-({3-Chloro-4-[(1-methyl-1H-imidazol-2-yl)sulfanyl]phenyl}amino)-7-[6-(1-piperidinyl)methyl)-3-pyridinyl]-3-quinolinecarbonitrile, 4-({3-Chloro-4-[(1-methyl-1H-imidazol-2-yl)sulfanyl]phenyl}amino)-7-{6-[(4-isopropyl-1-piperazinyl)-methyl]-3-pyridinyl}-3-quinolinecarbonitrile, 4-(2,4-Dichloro-5-methoxyanilino)-7-[1-methyl-2-(4-morpholinylmethyl)-1H-imidazol-5-yl]-3-quinolinecarbonitrile, 4-(2,4-Dichloro-5-methoxyanilino)-7-{1-methyl-2-[(4-methyl-1-piperazinyl)methyl]-1H-imidazol-5-yl}-3-quinolinecarbonitrile, 4-(2,4-Dichloro-5-methoxyanilino)-7-(2-formyl-1-methyl-1H-imidazol-5-yl)-6-methoxy-3-quinolinecarbonitrile, 4-(2,4-Dichloro-5-methoxyanilino)-7-[4-({[2-(2-pyridinyl)ethyl]amino}-methyl)phenyl]-3-quinolinecarbo-nitrile, 4-(2,4-Dichloro-5-methoxyanilino)-7-(4-{[4-(2-hydroxyethyl)-1-piperazinyl]methyl}phenyl)-3-quinolinecarbonitrile, Methyl 1-{4-[3-cyano-4-(2,4-dichloro-5-methoxyanilino)-7-quinolinyl]benzyl}-4-piperidinecarboxylate, 4-(2,4-Dichloro-5-methoxyanilino)-6-methoxy-7-[1-methyl-2-(4-morpholinylmethyl)-1H-imidazol-5-yl]-3-quinolinecarbonitrile, 4-(2,4-Dichloro-5-methoxyanilino)-6-methoxy-7-{1-methyl-2-[(4-methyl-1-piperazinyl)methyl]-1H-imidazol-5-yl}-3-quinolinecarbonitrile, 4-(2-Chloro-5-methoxy-4-methylanilino)-7-[4-(4-morpholinylmethyl)phenyl]-3-quinolinecarbonitrile, 4-(2-Chloro-4-fluoro-5-methoxyanilino)-7-[4-(4-morpholinylmethyl)phenyl]-3-quinolinecarbonitrile, 4-(2-Chloro-5-methoxyanilino)-7-[4-(4-morpholinylmethyl)-phenyl]-3-quinolinecarbonitrile, 1-{4-[3-Cyano-4-(2,4-dichloro-5-methoxyanilino)-7-quinolinyl]benzyl}-4-piperidinecarboxylic acid, 4-{3-chloro-4-[(1-methyl-1H-imidazol-2-yl)sulfanyl]anilino}-7-{4-[(4-methyl-1-piperazinyl)methyl]phenyl}-3-quinolinecarbo-nitrile, 4-{3-chloro-4-[(1-methyl-1H-imidazol-2-yl)sulfanyl]anilino}-7-[4-({[2-(dimethylamino)ethyl]amino}methyl)phenyl]-3-quinolinecarbonitrile, 4-{3-chloro-4-[(1-methyl-1H-imidazol-2-yl)sulfanyl]anilino}-7-(4-{[4-(1-pyrrolidinyl)-1-piperidinyl]-methyl}phenyl)-3-quinolinecarbonitrile, 4-{3-chloro-4-[(1-methyl-1H-imidazol-2-yl)sulfanyl]anilino}-7-(4-{[(4-pyridin ylmethyl)amino]methyl}phenyl)-3-quinolinecarbonitrile, 4-{3-chloro-4-[(1-methyl-1H-imidazol-2-yl)sulfanyl]anilino}-7-{4-[(dimethylamino)methyl]phenyl}-3-quinolinecarbonitrile, 4-{3-chloro-4-[(1-methyl-1H-imidazol-2-yl)sulfanyl]anilino}-7-(4-{[(2-hydroxy ethyl)amino]methyl}phenyl)-3-quinolinecarbonitrile, 4-{3-chloro-4-[(1-methyl-1H-imidazol-2-yl)sulfanyl]anilino}-7-[4-({[3-(4-morpholinyl)propyl]amino}-methyl)phenyl]-3-quinolinecarbonitrile, 4-{3-chloro-4-[(1-methyl-1H-imidazol-2-yl)sulfanyl]anilino}-7-{4-[(4-ethyl-1-piperazinyl)methyl]phenyl}-3-quinolinecarbonitrile, 4-{3-chloro-4-[(1-methyl-1H-imidazol-2-yl)sulfanyl]anilino}-7-(4-{[4-(2-hydroxyethyl)-1-piperazinyl]methyl}phenyl)-3-quinolinecarbonitrile, 4-{3-chloro-4-[(1-methyl-1H-imidazol-2-yl)sulfanyl]anilino}-7-{3-[(4-hydroxy-1-piperidinyl)methyl]phenyl}-3-quinolinecarbonitrile, 4-{3-chloro-4-[(1-methyl-1H-imidazol-2-yl)sulfanyl]anilino}-7-[3-({[2-(dimethylamino)ethyl]amino}methyl)phenyl]-3-quinolinecarbonitrile, 4-{3-chloro-4-[(1-methyl-1H-imidazol-2-yl)sulfanyl]anilino}-7-(3-{[4-(1-pyrrolidinyl)-1-piperidinyl]-methyl}phenyl)-3-quinolinecarbonitrile, 4-{3-chloro-4-[(1-methyl-1H-imidazol-2-yl)sulfanyl]anilino}-7-(3-{[(4-pyridinylmethyl)amino]methyl}phenyl)-3-quinolinecarbo-nitrile, 4-{3-chloro-4-[(1-methyl-1H-imidazol-2-yl)sulfanyl]anilino}-7-{3-[(dimethylamino)methyl]phenyl}-3-quinolinecarbonitrile, 4-{3-chloro-4-[(1-methyl-1H-imidazol-2-yl)sulfanyl]anilino}-7-[3-(4-morpholin ylmethyl)phenyl]-3-quinolinecarbonitrile, 4-{3-chloro-4-[(1-methyl-1H-imidazol-2-yl)sulfanyl]anilino}-7-(3-{[(2-hydroxyethyl)amino]-methyl}phenyl)-3-quinolinecarbonitrile, 4-{3-chloro-4-[(1-methyl-1H-imidazol-2-yl)sulfanyl]anilino}-7-{3-[(4-methyl-1-piperazinyl)methyl]phenyl}-3-quinolinecarbonitrile, 4-{3-chloro-4-[(1-methyl-1H-imidazol-2-yl)sulfanyl]anilino}-7-[3-({[3-(4-morpholinyl)propyl]amino}methyl)phenyl]-3-quinolinecarbonitrile, 4-{3-chloro-4-[(1-methyl-1H-imidazol-2-yl)sulfanyl]anilino}-7-[3-(1-piperidinyl)methyl)phenyl]-3-quinolinecarbonitrile, 4-{3-chloro-4-[(1-methyl-1H-imidazol-2-yl)sulfanyl]anilino}-7-{3-[(4-ethyl-1-piperazinyl)methyl]phenyl}-3-quinolinecarbonitrile, 4-{3-chloro-4-[(1-methyl-1H-imidazol-2-yl)sulfanyl]anilino}-7-(3-{[4-(2-hydroxyethyl)-1-piperazinyl]-methyl}phenyl)-3-quinolinecarbonitrile, 4-{3-chloro-4-[(1-methyl-1H-imidazol-2-yl)sulfanyl]anilino}-7-{5-[(4-hydroxy-1-piperidinyl)methyl]-2-furyl}-3-quinolinecarbo nitrile, 4-{3-chloro-4-[(1-methyl-1H-imidazol-2-yl)sulfanyl]anilino}-7-[5-({[2-(dimeth ylamino)ethyl]amino}methyl)-2-furyl]-3-quinolinecarbonitrile, 4-{3-chloro-4-[(1-methyl-1H-imidazol-2-yl)sulfanyl]anilino}-7-(5-{[4-(1-pyrrolidinyl)-1-piperidinyl]methyl}-2-furyl)-3-quinolinecarbonitrile, 4-{3-chloro-4-[(1-methyl-1H-imidazol-2-yl)sulfanyl]-anilino}-7-(5-{[(2-hydroxy ethyl)amino]methyl}-2-furyl)-3-quinolinecarbonitrile, 4-{3-chloro-4-[(1-methyl-1H-imidazol-2-yl)sulfanyl]anilino}-7-{5-[(4-methyl-1-piperazinyl)-methyl]-2-furyl}-3-quinolinecarbonitrile, 4-{3-chloro-4-[(1-methyl-1H-imidazol-2-yl)sulfanyl]anilino}-7-[5-({[3-(4-morpholinyl)propyl]amino}methyl)-2-furyl]-3-quinolinecarbonitrile, 4-{3-chloro-4-[(1-methyl-1H-imidazol-2-yl)sulfanyl]anilino}-7-[5-({[2-(dimethylamino)ethyl]amino}methyl)-2-thienyl]-3-quinolinecarbonitrile, 4-{3-chloro-4-[(1-methyl-1H-imidazol-2-yl)sulfanyl]anilino}-7-(5-{[4-(1-pyrrolidinyl)-1-piperidinyl]-methyl}-2-thienyl)-3-quinolinecarbonitrile, 4-{3-chloro-4-[(1-methyl-1H-imidazol-2-yl)sulfanyl]anilino}-7-(5-{[(2-hydroxyethyl)amino]methyl}-2-thienyl)-3-quinolinecarbonitrile, 4-{3-chloro-4-[(1-methyl-1H-imidazol-2-yl)sulfanyl]anilino}-7-[5-({[3-(4-morpholinyl)propyl]amino}methyl)-2-thienyl]-3-quinolinecarbonitrile, 4-{3-chloro-4-[(1-methyl-1H-imidazol-2-yl)sulfanyl]anilino}-7-{5-[(4-ethyl-1-piperazinyl)methyl]-2-thienyl}-3-quinolinecarbonitrile, 4-{3-chloro-4-[(1-methyl-1H-imidazol-2-yl)sulfanyl]-anilino}-7-[5-({[2-(dimethylamino)ethyl]amino}methyl)-2-pyridinyl]-3-quinoline-carbonitrile, 4-{3-chloro-4-[(1-methyl-1H-imidazol-2-yl)sulfanyl]anilino}-7-[5-({[3-(4-morpholinyl)propyl]amino}methyl)-2-pyridinyl]-3-quinolinecarbonitrile, 4-(2,4-dimethylanilino)-7-{4-[(4-hydroxy-1-piperidinyl)methyl]phenyl}-3-quinolinecarbonitrile, 7-[4-({[2-(dimethylamino)ethyl]amino}methyl)phenyl]-4-(2,4-dimethylanilino)-3-quinolinecarbonitrile, 4-(2,4-dimethylanilino)-7-(4-{[4-(1-pyrrolidinyl)-1-piperidinyl]-methyl}phenyl)-3-quinolinecarbonitrile, 4-(2,4-dimethylanilino)-7-(4-{[(4-pyridinyl-methyl)amino]methyl}phenyl)-3-quinolinecarbonitrile, 4-(2,4-dimethylanilino)-7-[4-({[2-(1H-imidazol-4-yl)ethyl]amino}methyl)phenyl]-3-quinolinecarbonitrile, 4-(2,4-dimethylanilino)-7-[4-(4-morpholinylmethyl)phenyl]-3-quinolinecarbonitrile, 4-(2,4-dimethylanilino)-7-(4-{[(2-hydroxyethyl)amino]methyl}phenyl)-3-quinolinecarbonitrile, 4-(2,4-dimethylanilino)-7-{4-[(4-methyl-1-piperazinyl)methyl]phenyl}-3-quinolinecarbo-nitrile, 4-(2,4-dimethylanilino)-7-[4-({[3-(4-morpholinyl)propyl]amino}methyl)phenyl]-3-quinolinecarbonitrile, 4-(2,4-dimethylanilino)-7-[4-(1-piperidinyl)methyl)phenyl]-3-quinolinecarbonitrile, 4-(2,4-dimethylanilino)-7-{4-[(4-ethyl-1-piperazinyl)methyl]-phenyl}-3-quinolinecarbonitrile, 4-(2,4-dimethylanilino)-7-(4-{[4-(2-hydroxyethyl)-1-piperazinyl]methyl}phenyl)-3-quinolinecarbonitrile, 4-(2,4-dimethylanilino)-7-{3-[(4-hydroxy-1-piperidinyl)methyl]phenyl}-3-quinolinecarbonitrile, 7-[3-({[2-(dimethyl-amino)ethyl]amino}methyl)phenyl]-4-(2,4-dimethylanilino)-3-quinolinecarbonitrile, 4-(2,4-dimethylanilino)-7-(3-{[4-(1-pyrrolidinyl)-1-piperidinyl]methyl}phenyl)-3-quinolinecarbonitrile, 4-(2,4-dimethylanilino)-7-(3-{[(4-pyridinylmethyl)amino]methyl}-phenyl)-3-quinolinecarbonitrile, 4-(2,4-dimethylanilino)-7-[3-({[2-(1H-imidazol-4-yl)ethyl]amino}methyl)phenyl]-3-quinolinecarbonitrile, 4-(2,4-dimethylanilino)-7-[3-(4-morpholinylmethyl)phenyl]-3-quinolinecarbonitrile, 4-(2,4-dimethylanilino)-7-(3-{[(2-hydroxyethyl)amino]methyl}phenyl)-3-quinolinecarbonitrile, 4-(2,4-dimethylanilino)-7-{3-[(4-methyl-1-piperazinyl)methyl]phenyl}-3-quinolinecarbonitrile, 4-(2,4-dimethylanilino)-7-[3-({[3-(4-morpholinyl)propyl]amino}methyl)phenyl]-3-quinoline-carbonitrile, 4-(2,4-dimethylanilino)-7-[3-(1-piperidinyl)methyl)phenyl]-3-quinoline-carbonitrile, 4-(2,4-dimethyl-anilino)-7-{3-[(4-ethyl-1-piperazinyl)methyl]-phenyl}-3-quinolinecarbonitrile, 4-(2,4-dimethylanilino)-7-(3-{[4-(2-hydroxyethyl)-1-piperazinyl]-methyl}phenyl)-3-quinolinecarbonitrile, 4-(2,4-dimethylanilino)-7-{5-[(4-hydroxy-1-piperidinyl)methyl]-2-furyl}-3-quinolinecarbonitrile, 7-[5-({[2-(dimethylamino)ethyl]-amino}methyl)-2-furyl]-4-(2,4-dimethylanilino)-3-quinolinecarbonitrile, 4-(2,4-dimethylanilino)-7-(5-{[4-(1-pyrrolidinyl)-1-piperidinyl]methyl}-2-furyl)-3-quinoline-carbonitrile, 4-(2,4-dimethylanilino)-7-(5-{[(4-pyridinylmethyl)amino]methyl}-2-furyl)-3-quinolinecarbonitrile, 4-(2,4-dimethylanilino)-7-[5-({[2-(1H-imidazol-4-yl)ethyl]amino}-methyl)-2-furyl)-3-quinolinecarbonitrile, 4-(2,4-dimethylanilino)-7-[5-(4-morpholinyl-methyl)-2-furyl]-3-quinolinecarbonitrile, 4-(2,4-dimethylanilino)-7-(5-{[(2-hydroxy-ethyl)amino]methyl}-2-furyl)-3-quinolinecarbonitrile, 4-(2,4-dimethylanilino)-7-{5-[(4-methyl-1-piperazinyl)methyl]-2-furyl}-3-quinolinecarbonitrile, 4-(2,4-dimethylanilino)-7-[5-(1-piperidinyl)methyl)-2-furyl]-3-quinolinecarbonitrile, 4-(2,4-dimethylanilino)-7-{5-[(4-ethyl-1-piperazinyl)methyl]-2-furyl}-3-quinolinecarbonitrile, 4-(2,4-dimethylanilino)-7-(5-{[4-(2-hydroxyethyl)-1-piperazinyl]methyl}-2-furyl)-3-quinolinecarbonitrile, 4-(2,4-dimethylanilino)-7-{5-[(4-hydroxy-1-piperidinyl)methyl]-2-thienyl}-3-quinolinecarbo-nitrile, 7-[5-({[2-(dimethylamino)ethyl]amino}methyl)-2-thienyl]-4-(2,4-dimethylanilino)-3-quinolinecarbonitrile, 4-(2,4-dimethylanilino)-7-(5-{[4-(1-pyrrolidinyl)-1-piperidinyl]methyl}-2-thienyl)-3-quinolinecarbonitrile, 4-(2,4-dimethylanilino)-7-(5-{[(4-pyridinylmethyl)amino]methyl}-2-thienyl)-3-quinolinecarbonitrile, 4-(2,4-dimethyl-anilino)-7-[5-({[2-(1H-imidazol-4-yl)ethyl]amino}methyl)-2-thienyl]-3-quinoline-carbonitrile, 4-(2,4-dimethylanilino)-7-[5-(4-morpholinylmethyl)-2-thienyl]-3-quinoline-carbonitrile, 4-(2,4-dimethylanilino)-7-(5-{[(2-hydroxyethyl)amino]methyl}-2-thienyl)-3-quinolinecarbonitrile, 4-(2,4-dimethylanilino)-7-{5-[(4-methyl-1-piperazinyl)-methyl]-2-thienyl}-3-quinolinecarbonitrile, 4-(2,4-dimethylanilino)-7-{5-[(4-ethyl-1-piperazinyl)-methyl]-2-thienyl}-3-quinolinecarbonitrile, 4-(2,4-dimethylanilino)-7-(5-{[4-(2-hydroxyethyl)-1-piperazinyl]methyl}-2-thienyl)-3-quinolinecarbonitrile, 4-(2,4-dimethylanilino)-7-{5-[(4-hydroxy-1-piperidinyl)methyl]-3-thienyl}-3-quinolinecarbo-nitrile, 7-[5-({[2-(dimethylamino)ethyl]amino}methyl)-3-thienyl]-4-(2,4-dimethylanilino)-3-quinolinecarbonitrile, 4-(2,4-dimethylanilino)-7-(5-{[4-(1-pyrrolidinyl)-1-piperidinyl]-methyl}-3-thienyl)-3-quinolinecarbonitrile, 4-(2,4-dimethylanilino)-7-(5-{[(4-pyridinyl-methyl)amino]methyl}-3-thienyl)-3-quinolinecarbonitrile, 4-(2,4-dimethylanilino)-7-[5-({[2-(1H-imidazol-4-yl)ethyl]amino}methyl)-3-thienyl]-3-quinolinecarbonitrile, 4-(2,4-dimethylanilino)-7-[5-(4-morpholinylmethyl)-3-thienyl]-3-quinolinecarbonitrile, 4-(2,4-dimethylanilino)-7-(5-{[(2-hydroxyethyl)amino]methyl}-3-thienyl)-3-quinolinecarbo-nitrile, 4-(2,4-dimethylanilino)-7-{5-[(4-methyl-1-piperazinyl)methyl]-3-thienyl}-3-quinolinecarbonitrile, 4-(2,4-dimethylanilino)-7-[5-({[3-(4-morpholinyl)propyl]amino}-methyl)-3-thienyl]-3-quinolinecarbonitrile, 4-(2,4-dimethylanilino)-7-[5-(1-piperidinyl-methyl)-3-thienyl]-3-quinolinecarbonitrile, 4-(2,4-dimethylanilino)-7-{5-[(4-ethyl-1-piperazinyl)methyl]-3-thienyl}-3-quinolinecarbonitrile, 4-(2,4-dimethylanilino)-7-(5-{[4-(2-hydroxyethyl)-1-piperazinyl]methyl}-3-3-quinolinecarbonitrile, 4-(4-bromo-2-chloro-6-methylanilino)-7-{5-[(4-hydroxy-1-piperidinyl)methyl]-2-furyl}-3-quinolinecarbonitrile, 4-(4-bromo-2-chloro-6-methylanilino)-7-[5-({[2-(dimethylamino)ethyl]amino}methyl)-2-furyl]-3-quinolinecarbonitrile, 4-(4-bromo-2-chloro-6-methylanilino)-7-(5-{[4-(1-pyrrolidinyl)-1-piperidinyl]methyl}-2-furyl)-3-quinolinecarbonitrile, 4-(4-bromo-2-chloro-6-methylanilino)-7-(5-{[(4-pyridinylmethyl)amino]methyl}-2-furyl)-3-quinolinecarbo-nitrile, 4-(4-bromo-2-chloro-6-methylanilino)-7-[5-({[2-(1H-imidazol-4-yl)ethyl]amino}-methyl)-2-furyl]-3-quinolinecarbonitrile, 4-(4-bromo-2-chloro-6-methylanilino)-7-[5-(4-morpholinylmethyl)-2-furyl]-3-quinolinecarbonitrile, 4-(4-bromo-2-chloro-6-methyl-anilino)-7-(5-{[(2-hydroxyethyl)amino]methyl}-2-furyl)-3-quinolinecarbonitrile, 4-(4-bromo-2-chloro-6-methylanilino)-7-{5-[(4-methyl-1-piperazinyl)methyl]-2-furyl}-3-quinolinecarbonitrile, 4-(4-bromo-2-chloro-6-methylanilino)-7-[5-({[3-(4-morpholinyl)-propyl]amino}methyl)-2-furyl]-3-quinolinecarbonitrile, 4-(4-bromo-2-chloro-6-methylanilino)-7-[5-(1-piperidinyl)methyl)-2-furyl]-3-quinolinecarbonitrile, 4-(4-bromo-2-chloro-6-methylanilino)-7-{5-[(4-ethyl-1-piperazinyl)methyl]-2-fury)}-3-quinoline-carbonitrile, 4-(4-bromo-2-chloro-6-methylanilino)-7-(5-{[4-(2-hydroxyethyl)-1-piperazinyl]methyl}-2-furyl)-3-quinoline-carbonitrile, 4-(4-bromo-2-chloro-6-methylanilino)-7-{5-[(4-hydroxy-1-piperidinyl)methyl]-2-thienyl}-3-quinolinecarbonitrile, 4-(4-bromo-2-chloro-6-methylanilino)-7-[5-({[2-(dimethylamino)ethyl]amino}methyl)-2-thienyl]-3-quinolinecarbonitrile, 4-(4-bromo-2-chloro-6-methylanilino)-7-(5-{[4-(1-pyrrolidinyl)-1-piperidinyl]methyl}-2-thienyl)-3-quinolinecarbonitrile, 4-(4-bromo-2-chloro-6-methylanilino)-7-(5-{[(4-pyridinylmethyl)amino]methyl}-2-thienyl)-3-quinolinecarbonitrile, 4-(4-bromo-2-chloro-6-methylanilino)-7-[5-({[2-(1H-imidazol-4-yl)ethyl]amino}methyl)-2-thienyl]-3-quinolinecarbonitrile, 4-(4-bromo-2-chloro-6-methylanilino)-7-[5-(4-morpholinyl-methyl)-2-thienyl]-3-quinolinecarbonitrile, 4-(4-bromo-2-chloro-6-methylanilino)-7-(5-{[(2-hydroxyethyl)amino]methyl}-2-thienyl)-3-quinolinecarbonitrile, 4-(4-bromo-2-chloro-6-methylanilino)-7-{5-[(4-methyl-1-piperazinyl)methyl]-2-thienyl}-3-quinoline-carbonitrile, 4-(4-bromo-2-chloro-6-methylanilino)-7-[5-({[3-(4-morpholinyl)-propyl]amino}-methyl)-2-thienyl]-3-quinoline-carbonitrile, 4-(4-bromo-2-chloro-6-methylanilino)-7-[5-(1-piperidinyl)methyl)-2-thienyl]-3-quinolinecarbonitrile, 4-({3-chloro-4′-[(4-hydroxy-1-piperidinyl)methyl]-5-methyl[1,1′-biphenyl]-4-yl}amino)-7-{4-[(4-hydroxy-1-piperidinyl)methyl]phenyl}-3-quinolinecarbonitrile, 4-{[3-chloro-4′-({[2-(dimethylamino)ethyl]amino}methyl)-5-methyl[1,1′-biphenyl]-4-yl]amino}-7-[4-({[2-(dimethylamino)ethyl]amino}methyl)-phenyl]-3-quinolinecarbonitrile, 4-[(3-chloro-5-methyl-4′-{[4-(1-pyrrolidinyl)-1-piperidinyl]methyl}[1,1′-biphenyl]-4-yl)amino]-7-(4-{[4-(1-pyrrolidinyl)-1-piperidinyl]-methyl}phenyl)-3-quinolinecarbonitrile, 4-{[3-chloro-5-methyl-4′-(4-morpholinyl-methyl)[1,1′-biphenyl]-4-yl]amino}-7-[4-(4-morpholinyl-methyl)phenyl]-3-quinoline-carbonitrile, 4-[(3-chloro-4′-{[(2-hydroxyethyl)amino]-methyl}-5-methyl[1,1′-biphenyl]-4-yl)amino]-7-(4-{[(2-hydroxyethyl)amino]methyl phenyl)-3-quinolinecarbonitrile, 4-({3-chloro-5-methyl-4′-[(4-methyl-1-piperazinyl)-methyl][1,1′-biphenyl]-4-yl}amino)-7-{4-[(4-methyl-1-piperazinyl)methyl]phenyl}-3-quinolinecarbonitrile, 4-{[3-chloro-5-methyl-4′-({[3-(4-morpholinyl)propyl]amino}-methyl)[1,1′-biphenyl]-4-yl]amino}-7-[4-({[3-(4-morpholinyl)propyl]amino}methyl)-phenyl]-3-quinolinecarbonitrile, 4-{[3-chloro-5-methyl-4′-(1-piperidinyl)methyl)[1,1′-biphenyl]-4-yl]amino}7-[4-(1-piperidinyl-methyl)phenyl]-3-quinolinecarbonitrile, 4-({3-chloro-4′-[(4-ethyl-1-piperazinyl)methyl]-5-methyl[1,1′-biphenyl]-4-yl}amino)-7-{4-[(4-ethyl-1-piperazinyl)methyl]phenyl}-3-quinolinecarbonitrile, 4-[(3-chloro-4′-{[4-(2-hydroxyethyl)-1-piperazinyl]methyl}-5-methyl[1,1′-biphenyl]-4-yl)amino]-7-(4-{[4-(2-hydroxyethyl)-1-piperazinyl]methyl}phenyl)-3-quinolinecarbonitrile, 4-({3-chloro-3′-[(4-hydroxy-1-piperidinyl)methyl]-5-methyl[1,1′-biphenyl]-4-yl}amino)-7-{3-[(4-hydroxy-1-piperidinyl)methyl]phenyl}-3-quinolinecarbonitrile, 4-{[3-chloro-3′-({[2-(dimethyl-amino)ethyl]amino}methyl)-5-methyl[1,1′-biphenyl]-4-yl]amino}-7-[3-({[2-(dimethylamino)ethyl]amino}methyl)-phenyl]-3-quinolinecarbonitrile, 4-[(3-chloro-5-methyl-3′-{[4-(1-pyrrolidinyl)-1-piperidinyl]methyl}[1,1′-biphenyl]-4-yl)amino]-7-(3-{[4-(1-pyrrolidinyl)-1-piperidinyl]methyl}phenyl)-3-quinolinecarbonitrile, 4-[(3-chloro-5-methyl-3′-{[(4-pyridinylmethyl)amino]methyl}[1,1′-biphenyl]-4-yl)amino]-7-(3-{[(4-pyridinylmethyl)-amino]methyl}phenyl)-3-quinolinecarbonitrile, 4-{[3-chloro-3′-({[2-(1H-imidazol-4-yl)ethyl]amino}methyl)-5-methyl[1,1′-biphenyl]-4-yl]amino}-7-[3-({[2-(1H-imidazol-4-yl)ethyl]amino}methyl)phenyl]-3-quinolinecarbonitrile, 4-{[3-chloro-5-methyl-3′-(4-morpholinylmethyl)[1,1′-biphenyl]-4-yl]amino}-7-[3-(4-morpholinylmethyl)phenyl]-3-quinolinecarbonitrile, 4-[(3-chloro-3′-{[(2-hydroxyethyl)amino]methyl}-5-methyl[1,1′-biphenyl]-4-yl)amino]-7-(3-{[(2-hydroxyethyl)amino]methyl}phenyl)-3-quinoline-carbonitrile, 4-({3-chloro-5-methyl-3′-[(4-methyl-1-piperazinyl)methyl)[1,1′-biphenyl]-4-yl}amino)-7-{3-[(4-methyl-1-piperazinyl)methyl]phenyl}-3-quinolinecarbonitrile, 4-{[3-chloro-5-methyl-3′-({[3-(4-morpholinyl)propyl]amino}methyl)[1,1-biphenyl]-4-yl]amino}-7-[3-({[3-(4-morpholinyl)propyl]amino}methyl)phenyl]-3-quinolinecarbonitrile, 4-{[3-chloro-5-methyl-3′-(1-piperidinyl)methyl)[1,1′-biphenyl]-4-yl]amino}-7-[3-(1-piperidinyl-methyl)phenyl]-3-quinolinecarbonitrile, 4-({3-chloro-3′-[(4-ethyl-1-piperazinyl)methyl]-5-methyl[1,1′-biphenyl]-4-yl}amino)-7-{3-[(4-ethyl-1-piperazinyl)methyl]phenyl}-3-quinolinecarbonitrile, 4-[(3-chloro-3′-{[4-(2-hydroxyethyl)-1-piperazinyl]methyl}-5-methyl[1,1′-biphenyl]-4-yl)amino]-7-(3-{[4-(2-hydroxyethyl)-1-piperazinyl]methyl}phenyl)-3-quinolinecarbonitrile, 4-{2-chloro-4-[5-({[2-(dim ethylamino)ethyl]-amino}methyl)-3-thienyl]-6-methylanilino}-7-[5-({[2-(dimethylamino)ethyl]amino}methyl)-3-thienyl]-3-quinolinecarbonitrile, 4-[2-chloro-6-methyl-4-(5-{[4-(1-pyrrolidinyl)-1-piperidinyl]methyl}-3-thienyl)anilino]-7-(5-{[4-(1-pyrrolidinyl)-1-piperidinyl]methyl}-3-quinolinecarbonitrile, 4-[2-chloro-6-methyl-4-(5-{[(4-pyridinylmethyl)amino]methyl}-3-thienyl)anilino]-7-(5-{[(4-pyridinylmethyl)amino]methyl}-3-thienyl)-3-quinolinecarbo-nitrile, 4-{2-chloro-4-[5-({[2-(1H-imidazol-4-yl)ethyl]amino}methyl)-3-thienyl]-6-methylanilino}-7-[5-({[2-(1H-imidazol-4-yl)ethyl]amino}methyl)-3-thienyl]-3-quinolinecarbo-nitrile, 4-{2-chloro-6-methyl-4-[5-(4-morpholinylmethyl)-3-thienyl]anilino}-7-[5(4-morpholinylmethyl)-3-thienyl]-3-quinolinecarbonitrile, 4-(2-chloro-6-methyl-4-{5-[(4-methyl-1-piperazinyl)methyl]-3-thienyl}anilino)-7-{5-[(4-methyl-1-piperazinyl)methyl]-3-thienyl}-3-quinolinecarbonitrile, 4-{2-chloro-6-methyl-4-[5-({[3-(4-morpholinyl)-propyl]amino}methyl)-3-thienyl]anilino}-7-[5-({[3-(4-morpholinyl)propyl]amino}methyl)-3-thienyl]-3-quinolinecarbonitrile, 4-{2-chloro-6-methyl-4-[5-(1-piperidinyl-methyl)-3-thienyl]anilino}-7-[5-(1-piperidinyl)methyl)-3-thienyl]-3-quinolinecarbonitrile, 4-(2-chloro-4-{5-[(4-ethyl-1-piperazinyl)methyl]-3-thienyl}-6-methylanilino)-7-{5-[(4-ethyl-1-piperazinyl)methyl]-3-thienyl}-3-quinolinecarbonitrile, 4-[2-chloro-4-(5-{[4-(2-hydroxyethyl)-1-piperazinyl]methyl}-3-thienyl)-6-methylanilino]-7-(5-{[4-(2-hydroxyethyl)-1-piperazinyl]methyl}-3-thienyl)-3-quinolinecarbonitrile, 4-[(3-chloro-4-phenoxyphenyl)amino]-7-[4-({[3-(4-morpholinyl)propyl]amino}-methyl)phenyl]-3-quinolinecarbonitrile, 4-[(3-chloro-4-phenoxyphenyl)amino]-7-{4-[(4-ethyl-1-piperazinyl)methyl]phenyl}-3-quinolinecarbonitrile, 4-[(3-chloro-4-phenoxyphenyl)-amino]-7-(4-{[4-(2-hydroxyethyl)-1-piperazinyl]methyl}phenyl)-3-quinolinecarbonitrile, 4-[(3-chloro-4-phenoxyphenyl)amino]-7-[3-({[2-(dimethylamino)ethyl]amino}methyl)-phenyl]-3-quinolinecarbonitrile, 4-[(3-chloro-4-phenoxyphenyl)amino]-7-(3-{[4-(1-pyrrolidinyl)-1-piperidinyl]methyl}phenyl)-3-quinolinecarbonitrile, 4-[(3-chloro-4-phenoxyphenyl)amino]-7-(3-{[(4-pyridinylmethyl)amino]methyl}phenyl)-3-quinoline-carbonitrile, 4-[(3-chloro-4-phenoxyphenyl)amino]-7-[3-({[3-(4-morpholinyl)propyl]amino}methyl)phenyl]-3-quinolinecarbonitrile, 4-[(3-chloro-4-phenoxyphenyl)amino]-7-{3-[(4-ethyl-1-piperazinyl)methyl]phenyl}-3-quinolinecarbonitrile, 4-[(3-chloro-4-phenoxyphenyl)amino]-7-{5-[(4-hydroxy-1-piperidinyl)methyl]-2-furyl}-3-quinoline-carbonitrile, 4-[(3-chloro-4-phenoxyphenyl)amino]-7-[5-({[2-(dimethylamino)ethyl]amino}methyl)-2-furyl]-3-quinolinecarbonitrile, 4-[(3-chloro-4-phenoxyphenyl)amino]-7-(5-{[4-(1-pyrrolidinyl)-1-piperidinyl]methyl}-2-furyl)-3-quinolinecarbonitrile, 4-[(3-chloro-4-phenoxyphenyl)amino]-7-(5-{[(4-pyridinylmethyl)amino]methyl}-2-furyl)-3-quinolinecarbonitrile, 4-[(3-chloro-4-phenoxyphenyl)amino]-7-[5-({[2-(1H-imidazol-4-yl)ethyl]amino}methyl)-2-furyl]-3-quinolinecarbonitrile, 4-[(3-chloro-4-phenoxyphenyl)-amino]-7-[5-(4-morpholinylmethyl)-2-furyl]-3-quinolinecarbonitrile, 4-[(3-chloro-4-phenoxyphenyl)amino]-7-(5-{[(2-hydroxyethyl)amino]methyl}-2-furyl)-3-quinoline-carbonitrile, 4-[(3-chloro-4-phenoxyphenyl)amino]-7-{5-[(4-methyl-1-piperazinyl0-methyl]-2-furyl}-3-quinolinecarbonitrile, 4-[(3-chloro-4-phenoxyphenyl)-amino]-7-[5-({[3-(4-morpholinyl)propyl]amino}methyl)-2-furyl]-3-quinolinecarbonitrile, 4-[(3-chloro-4-phenoxyphenyl)amino]-7-[5-(1-piperidinyl)methyl)-2-furyl]-3-quinolinecarbonitrile, 4-[(3-chloro-4-phenoxyphenyl)amino]-7-{5-[(4-ethyl-1-piperazinyl)methyl]-2-furyl}-3-quinolinecarbonitrile, 4-[(3-chloro-4-phenoxyphenyl)amino]-7-(5-({[4-(2-hydroxyethyl)-1-piperazinyl]methyl}-2-furyl)-3-quinolinecarbonitrile, 4-[(3-chloro-4-phenoxy-phenyl)amino]-7-[5-({[2-(dimethylamino)ethyl]amino}methyl)-2-thienyl]-3-quinoline-carbonitrile, 4-[(3-chloro-4-phenoxyphenyl)amino]-7-(5-{[4-(1-pyrrolidinyl)-1-piperidinyl]methyl}-2-thienyl)-3-quinolinecarbonitrile, 4-[(3-chloro-4-phenoxyphenyl)-amino]-7-(5-{[(4-pyridinylmethyl)amino]methyl}-2-thienyl)-3-quinolinecarbonitrile, 4-[(3-chloro-4-phenoxyphenyl)amino]-7-[5-(4-morpholinylmethyl)-2-thienyl]-3-quinoline-carbonitrile, 4-[(3-chloro-4-phenoxyphenyl)amino]-7-{5-[(4-methyl-1-piperazinyl)-methyl]-2-thienyl}-3-quinolinecarbonitrile, 4-[(3-chloro-4-phenoxyphenyl)-amino]-7-{5-[(4-ethyl-1-piperazinyl)methyl]-2-thienyl}-3-quinolinecarbonitrile, 4-[(3-chloro-4-phenoxyphenyl)amino]-7-{5-[(4-hydroxy-1-piperidinyl)methyl]-3-thienyl}-3-quinoline-carbonitrile, 4-[(3-chloro-4-phenoxyphenyl)amino]-7-[5-({[2-(dimethylamino)-ethyl]amino}methyl)-3-thienyl]-3-quinolinecarbonitrile, 4-[(3-chloro-4-phenoxy-phenyl)amino]-7-(5-{[4-(1-pyrrolidinyl)-1-piperidinyl]methyl}-3-thienyl)-3-quinolinecarbonitrile, 4-[(3-chloro-4-phenoxyphenyl)amino]-7-(5-{[(4-pyridinylmethyl)-amino]methyl}-3-thienyl)-3-quinolinecarbonitrile, 4-[(3-chloro-4-phenoxyphenyl)-amino]-7-[5-({[2-(1H-imidazol-4-yl)ethyl]amino}methyl)-3-thienyl]-3-quinolinecarbo-nitrile, 4-[(3-chloro-4-phenoxyphenyl)amino]-7-[5-(4-morpholinyl-methyl)-3-thienyl]-3-quinolinecarbonitrile, 4-[(3-chloro-4-phenoxyphenyl)amino]-7-(5-{[(2-hydroxyethyl)-amino]methyl}-3-thienyl)-3-quinolinecarbonitrile, 4-[(3-chloro-4-phenoxyphenyl)-amino]-7-{5-[(4-methyl-1-piperazinyl)methyl]-3-thienyl}-3-quinolinecarbonitrile, 4-[(3-chloro-4-phenoxyphenyl)amino]-7-[5-({[3-(4-morpholinyl)propyl]amino}methyl)-3-thienyl]-3-quinolinecarbonitrile, 4-[(3-chloro-4-phenoxyphenyl)amino]-7-[5-(1-piperidinyl-methyl)-3-thienyl]-3-quinolinecarbonitrile, 4-[(3-chloro-4-phenoxyphenyl)-amino]-7-{5-[(4-ethyl-1-piperazinyl)methyl]-3-thienyl}-3-quinolinecarbonitrile, 4-[(3-chloro-4-phenoxyphenyl)amino]-7-(5-{[4-(2-hydroxyethyl)-1-piperazinyl]methyl}-3-thienyl)-3-quinolinecarbonitrile, 4-[(3-chloro-4-phenoxyphenyl)amino]-7-[5-({[3-(4-morpholinyl)propyl]amino}methyl)-2-pyridinyl]-3-quinolinecarbonitrile, 4-{[3-chloro-4-(phenylsulfanyl)phenyl]amino}-7-{4-[(4-hydroxy-1-piperidinyl)methyl]phenyl}-3-quinolinecarbonitrile, 4-{[3-chloro-4-(phenylsulfanyl)phenyl]amino}-7-(4-{[(4-pyridinylmethyl)amino]methyl}phenyl)-3-quinolinecarbonitrile, 4-{[3-chloro-4-(phenylsulfanyl)phenyl]amino}-7-[4-({[2-(1H-imidazol-4-yl)ethyl]amino}methyl)phenyl]-3-quinolinecarbonitrile, 4-{[3-chloro-4-(phenylsulfanyl)phenyl]amino}-7-[4-(4-morpholinylmethyl)phenyl]-3-quinolinecarbonitrile, 4-{[3-chloro-4-(phenylsulfanyl)-phenyl]amino}-7-(4-{[(2-hydroxyethyl)amino]methyl}phenyl)-3-quinolinecarbonitrile, 4-{[3-chloro-4-(phenylsulfanyl)phenyl]amino}-7-{4-[(4-methyl-1-piperazinyl)-methyl]phenyl}-3-quinolinecarbonitrile, 4-{[3-chloro-4-(phenylsulfanyl)phenyl]amino}-7-[4-({[3-(4-morpholinyl) propyl]amino}methyl)phenyl]-3-quinolinecarbonitrile, 4-{[3-chloro-4-(phenylsulfanyl)phenyl]amino}-7-{4-[(4-ethyl-1-piperazinyl)methyl]phenyl}-3-quinolinecarbonitrile, 4-{[3-chloro-4-(phenylsulfanyl)phenyl]amino}-7-(4-{[4-(2-hydroxyethyl)-1-piperazinyl]methyl}phenyl)-3-quinolinecarbonitrile, 4-{[3-chloro-4-(phenylsulfanyl)phenyl]amino}-7-{3-[(4-hydroxy-1-piperidinyl)methyl]phenyl}-3-quinolinecarbonitrile, 4-{[3-chloro-4-(phenylsulfanyl)phenyl]amino}-7-[3-({[2-(dimethylamino)ethyl]amino}methyl)phenyl]-3-quinolinecarbonitrile, 4-{[3-chloro-4-(phenylsulfanyl)phenyl]amino}-7-(3-{[4-(1-pyrrolidinyl)-1-piperidinyl]methyl}phenyl)-3-quinolinecarbonitrile, 4-{[3-chloro-4-(phenylsulfanyl)phenyl]amino}-7-(3-{[(2-hydroxyethyl)amino]methyl}phenyl)-3-quinolinecarbonitrile, 4-{[3-chloro-4-(phenylsulfanyl)phenyl]amino}-7-[3-({[3-(4-morpholinyl) propyl]amino}methyl)phenyl]-3-quinolinecarbonitrile, 4-{[3-chloro-4-(phenylsulfanyl)phenyl]amino}-7-(3-{[4-(2-hydroxyethyl)-1-piperazinyl]methyl}phenyl)-3-quinolinecarbonitrile, 4-{[3-chloro-4-(phenylsulfanyl)phenyl]amino}-7-{5-[(4-hydroxy-1-piperidinyl)methyl]-2-furyl}-3-quinolinecarbonitrile, 4-{[3-chloro-4-(phenylsulfanyl)phenyl]amino}-7-[5-({[2-(dimethylamino)ethyl]amino}methyl)-2-furyl]-3-quinolinecarbonitrile, 4-{[3-chloro-4-(phenylsulfanyl)phenyl]amino}-7-(5-{[4-(1-pyrrolidinyl)-1-piperidinyl]methyl}-2-furyl)-3-quinolinecarbonitrile, 4-{[3-chloro-4-(phenylsulfanyl)phenyl]amino}-7-(5-{[(4-pyridinylmethyl)amino]methyl}-2-furyl)-3-quinolinecarbonitrile, 4-{[3-chloro-4-(phenylsulfanyl)phenyl]amino}-7-[5-({[2-(1H-imidazol-4-yl)ethyl]amino}methyl)-2-furyl]-3-quinolinecarbonitrile, 4-{[3-chloro-4-(phenylsulfanyl)phenyl]amino}-7-[5-(4-morpholinylmethyl)-2-furyl]-3-quinolinecarbonitrile, 4-{[3-chloro-4-(phenylsulfanyl)-phenyl]amino}-7-(5-{[(2-hydroxyethyl)amino]methyl}-2-furyl)-3-quinolinecarbonitrile, 4-{[3-chloro-4-(phenylsulfanyl)phenyl]amino}-7-{5-[(4-methyl-l1-piperazinyl)methyl]-2-fury)}-3-quinolinecarbonitrile, 4-{[3-chloro-4-(phenylsulfanyl)phenyl]amino}-7-[5-({[3-(4-morpholinyl) propyl]amino}methyl)-2-furyl]-3-quinolinecarbonitrile, 4-{[3-chloro-4-(phenylsulfanyl)phenyl]amino}-7-{5-[(4-ethyl-1-piperazinyl]methyl)-2-furyl}-3-quinolinecarbonitrile, 4-{[3-chloro-4-(phenylsulfanyl)phenyl]amino}-7-[5-({[2-(dimethylamino) ethyl]amino}methyl)-2-thienyl-3-quinolinecarbonitrile, 4-{[3-chloro-4-(phenylsulfanyl)phenyl]amino}-7-(5-{[4-(1-pyrrolidinyl)-1-piperidinyl]methyl}-2-thienyl)-3-quinolinecarbonitrile, 4-{[3-chloro-4-(phenylsulfanyl)phenyl]amino}-7-[5-(4-morpholinylmethyl)-2-thienyl]-3-quinolinecarbonitrile, 4-{[3-chloro-4-(phenylsulfanyl)-phenyl]amino}-7-{5-[(4-methyl-1-piperazinyl)methyl]-2-thienyl}-3-quinolinecarbonitrile, 4-{[3-chloro-4-(phenylsulfanyl)phenyl]amino}-7-[5-({[3-(4-morpholinyl) propyl]amino}-methyl)-2-thienyl]-3-quinolinecarbonitrile, 4-{[3-chloro-4-(phenylsulfanyl)phenyl]-amino}-7-{5-[(4-ethyl-1-piperazinyl)methyl]-2-thienyl}-3-quinolinecarbonitrile, 4-{[3-chloro-4-(phenylsulfanyl)phenyl]amino}-7-{5-[(4-hydroxy-1-piperidinyl)methyl]-3-thienyl}-3-quinolinecarbonitrile, 4-{[3-chloro-4-(phenylsulfanyl)-phenyl]amino}-7-[5-({[2-(dimethylamino)ethyl]amino}methyl)-3-thienyl]-3-quinolinecarbonitrile, 4-{[3-chloro-4-(phenylsulfanyl)phenyl]amino}-7-(5-{[(4-(1-pyrrolidinyl)-1-piperidinyl]methyl}-3-thienyl)-3-quinolinecarbonitrile, 4-{[3-chloro-4-(phenylsulfanyl)phenyl]amino}-7-(5-{[(4-pyridinylmethyl)amino]methyl}-3-thienyl)-3-quinolinecarbonitrile, 4-{[3-chloro-4-(phenylsulfanyl)phenyl]amino}-7-[5-({[2-(1H-imidazol-4-yl)ethyl]amino}-methyl)-3-thienyl]-3-quinolinecarbonitrile, 4-{[3-chloro-4-(phenylsulfanyl)phenyl]-amino}-7-[5-(4-morpholinylmethyl)-3-thienyl]-3-quinolinecarbonitrile, 4-{[3-chloro-4-(phenylsulfanyl)-phenyl]amino}-7-(5-{[(2-hydroxyethyl)aminomethyl}-3-thienyl)-3-quinolinecarbonitrile, 4-{[3-chloro-4-(phenylsulfanyl)-phenyl]amino}-7-{5-[(4-methyl-1-piperazinyl)methyl]3-3-thienyl}-3-quinolinecarbonitrile, 4-{[3-chloro-4-(phenylsulfanyl)-phenyl]amino}-7-[5-({[3-(4-morpholinyl)propyl]amino}methyl)-3-thienyl]-3-quinolinecarbonitrile, 4-{[3-chloro-4-(phenylsulfanyl)phenyl]amino}-7-[5-(1-piperidinyl)methyl)-3-thienyl]-3-quinolinecarbonitrile, 4-{[3-chloro-4-(phenylsulfanyl)-phenyl]amino}-7-{5-[(4-ethyl-1-piperazinyl)methyl]-3-thienyl}-3-quinolinecarbonitrile, 4-{[3-chloro-4-(phenylsulfanyl)-phenyl]amino}-7-(5-{[4-(2-hydroxyethyl)-1-piperazinyl]methyl}-3-thienyl)-3-quinolinecarbonitrile, 4-{[3-chloro-4-(3-furylmethyl)-phenyl]amino}-7-{4-[(4-hydroxy-1-piperidinyl)methyl]phenyl}-3-quinolinecarbonitrile, 4-{[3-chloro-4-(3-furylmethyl)-phenyl]amino}-7-[4-({[2-(dimethylamino) ethyl]amino}methyl)phenyl]-3-quinoline-carbonitrile, 4-{[3-chloro-4-(3-furylmethyl)-phenyl]amino}-7-(4-{[4-(1-pyrrolidinyl)-1-piperidinyl]methyl}phenyl)-3-quinolinecarbo-nitrile, 4-{[3-chloro-4-(3-furylmethyl)phenyl]amino}-7-(4-{[(4-pyridinylmethyl)amino]-methyl}phenyl)-3-quinolinecarbonitrile, 4-{[3-chloro-4-(3-furylmethyl)phenyl]amino}-7-[4-({[2-(1H-imidazol-4-yl)ethyl]amino}methyl)phenyl]-3-quinolinecarbonitrile, 4-{[3-chloro-4-(3-furylmethyl)phenyl]amino}-7-[4-(4-morpholinylmethyl)phenyl]-3-quinolinecarbonitrile, 4-{[3-chloro-4-(3-furylmethyl)phenyl]amino}-7-(4-{[(2-hydroxyethyl)aminomethyl}-phenyl)-3-quinolinecarbonitrile, 4-{[3-chloro-4-(3-furylmethyl)phenyl]amino}-7-{4-[(4-methyl-1-piperazinyl)methyl]phenyl}-3-quinolinecarbonitrile, 4-{[3-chloro-4-(3-furylmethyl)phenyl]amino}-7-[4-({[3-(4-morpholinyl) propyl]amino}methyl)phenyl]-3-quinolinecarbonitrile, 4-{[3-chloro-4-(3-furylmethyl)phenyl]amino}-7-[4-(1-piperidinyl-methyl)phenyl]-3-quinolinecarbonitrile, 4-{[3-chloro-4-(3-furylmethyl)phenyl]amino}-7-{4-[(4-ethyl-1-piperazinyl)methyl]phenyl}-3-quinolinecarbonitrile, 4-{[3-chloro-4-(3-furylmethyl)phenyl]amino}-7-(4-{[4-(2-hydroxyethyl)-1-piperazinyl]methyl}phenyl)-3-quinolinecarbonitrile, 4-{[4-(3-furylmethyl)phenyl]amino}-7-(4-{[(4-pyridinylmethyl)-amino]methyl}phenyl)-3-quinolinecarbonitrile, 4-{[4-(3-furylmethyl)phenyl]amino}-7-[4-({[2-(1H-imidazol-4-yl)ethyl]amino}methyl)phenyl]-3-quinolinecarbonitrile, 4-{[4-(3-furylmethyl)phenyl]amino}-7-[4-(4-morpholinylmethyl)phenyl)-3-quinolinecarbonitrile, 4-{[4-(3-furylmethyl)phenyl]amino}-7-(4-{[(2-hydroxyethyl)amino]methyl}phenyl)-3-quinolinecarbonitrile, 4-{[4-(3-furylmethyl)phenyl]amino}-7-{4-[(4-methyl-1-piperazinyl)methyl]phenyl}-3-quinolinecarbonitrile, 4-[{4-(3-furylmethyl)phenyl]amino}-7-[4-(1-piperidinyl)methyl)phenyl]-3-quinolinecarbonitrile, 7-{4-[(4-ethyl-1-piperazinyl)methyl]-phenyl}-4-{[4-(3-furylmethyl)phenyl]amino}-3-quinolinecarbonitrile, 4-{[4-(3-furylmethyl)phenyl]amino}-7-(4-{[4-(2-hydroxyethyl)-1-piperazinyl]methyl}phenyl)-3-quinolinecarbonitrile, 4-{[4-(3-furylmethyl)phenyl]amino}-7-{3-[(4-hydroxy-1-piperidinyl)methyl]phenyl}-3-quinolinecarbonitrile, 7-[3-({[2-(dimethylamino)-ethyl]amino}methyl)phenyl]-4-{[4-(3-furylmethyl)phenyl]amino}-3-quinolinecarbonitrile, 4-{[4-(3-furylmethyl)phenyl]amino}-7-(3-{[4-(1-pyrrolidinyl)-1-piperidinyl]methyl}-phenyl)-3-quinolinecarbonitrile, 4-{[4-(3-furylmethyl)phenyl]amino}-7-(3-{[(4-pyridinylmethyl)amino]methyl}phenyl)-3-quinolinecarbonitrile, 4-{[4-(3-furylmethyl)-phenyl]amino}-7-[3-({[2-(1H-imidazol-4-yl)ethyl]amino}methyl)phenyl]-3-quinoline-carbonitrile, 4-{[4-(3-furylmethyl)phenyl]amino}-7-[3-(4-morpholinyl-methyl)phenyl]-3-quinolinecarbonitrile, 4-{[4-(3-furylmethyl)phenyl]amino}-7-(3-{[(2-hydroxyethyl)-amino]methyl}phenyl)-3-quinolinecarbonitrile, 4-[4-(3-furylmethyl)phenyl]amino}-7-{3-[(4-methyl-1-piperazinyl)methyl]phenyl}-3-quinolinecarbonitrile, 4-{[4-(3-furylmethyl)-phenyl]amino}-7-[3-({[3-(4-morpholinyl)-propyl]amino}methyl)phenyl]-3-quinoline-carbonitrile, 7-{3-[(4-ethyl-1-piperazinyl)methyl]phenyl}-4-{[4-(3-furylmethyl)phenyl]amino}-3-quinolinecarbonitrile, 4-{[4-(3-furylmethyl)phenyl]amino}-7-(3-{[4-(2-hydroxyethyl)-1-piperazinyl]methyl}-phenyl)-3-quinolinecarbonitrile, 4-{[4-(3-furylmethyl)phenyl]amino}-7-{5-[(4-hydroxy-1-piperidinyl)methyl]-2-pyridinyl}-3-quinolinecarbonitrile, 7-[5-({[2-(dimethylamino)ethyl]amino}methyl)-2-pyridinyl]-4-{[4-(3-furylmethyl)phenyl]amino}-3-quinolinecarbonitrile, 4-{[4-(3-furylmethyl)phenyl]-amino}-7-(5-{[(2-hydroxyethyl)-aminomethyl}-2-pyridinyl)-3-quinolinecarbonitrile, 4-{[4-(3-furylmethyl)phenyl]amino}-7-[5-({[3-(4-morpholinyl)propyl]amino}methyl)-2-pyridinyl]-3-quinolinecarbonitrile, 4-{[4-(3-furylmethyl)phenyl]amino}-7-(5-{[4-(2-hydroxyethyl)-1-piperazinyl]methyl}-2-pyridinyl)-3-quinolinecarbonitrile, 4-{[4-(3-furylmethyl)phenyl]amino}-7-{5-[(4-hydroxy-1-piperidinyl)methyl]-3-thienyl}-3-quinolinecarbonitrile, 4-{[4-(3-furylmethyl)phenyl]-amino}-7-(5-{[4-(1-pyrrolidinyl)-1-piperidinyl]methyl}-3-thienyl)-3-quinolinecarbonitrile, 4-{[4-(3-furylmethyl)phenyl]-amino}-7-(5-{[(4-pyridinylmethyl)amino]methyl}-3-thienyl)-3-quinolinecarbonitrile, 4-{[4-(3-furylmethyl)phenyl]amino}-7-[5-(4-morpholinylmethyl)-3-thienyl]-3-quinoline-carbonitrile, 4-{[4-(3-furylmethyl)phenyl]amino}-7-(5-{[(2-hydroxyethyl)amino]methyl}-3-thienyl)-3-quinolinecarbonitrile, 4-{[4-(3-furylmethyl)phenyl]amino}-7-{5-[(4-methyl-1-piperazinyl)methyl]-3-thienyl}-3-quinolinecarbonitrile, 4-{[4-(3-furylmethyl)phenyl]-amino}-7-[5-({[3-(4-morpholinyl)-propyl]amino}methyl)-3-thienyl]-3-quinolinecarbo-nitrile, 4-{[4-(3-furylmethyl)phenyl]-amino}-7-[5-(1-piperidinyl)methyl)-3-thienyl]-3-quinolinecarbonitrile, 7-{5-[(4-ethyl-1-piperazinyl)methyl]-3-thienyl}-4-{[4-(3-furyl-methyl)phenyl]amino}-3-quinolinecarbonitrile, 4-{[4-(3-furylmethyl)phenyl]amino}-7-(5-{[4-(2-hydroxyethyl)-1-piperazinyl]methyl}-3-thienyl)-3-quinolinecarbonitrile, 4-(2,4-dichloro-5-methoxyanilino)-7-(4-{[4-(1-pyrrolidinyl)-1-piperidinyl]methyl}phenyl)-3-quinolinecarbonitrile, 4-(2,4-dichloro-5-methoxyanilino)-7-(4-{[(4-pyridinyl-methyl)amino]methyl}phenyl)-3-quinolinecarbo-nitrile, 4-(2,4-dichloro-5-methoxyanilino)-7-[4-({[2-(1H-imidazol-4-yl)ethyl]amino}methyl)phenyl]-3-quinolinecarbonitrile, 4-(2,4-dichloro-5-methoxyanilino)-7-(4-{[(2-hydroxyethyl)amino]methyl}phenyl)-3-quinolinecarbonitrile, 4-(2,4-dichloro-5-methoxyanilino)-7-[4-({[3-(4-morpholinyl)-propyl]amino}methyl)phenyl]-3-quinolinecarbonitrile, 4-(2,4-dichloro-5-methoxy-anilino)-7-{5-[(4-hydroxy-1-piperidinyl)methyl]-2-fury)}-3-quinolinecarbonitrile, 4-(2,4-dichloro-5-methoxyanilino)-7-[5-({[2-(dimethylamino)ethyl]amino}methyl)-2-furyl]-3-quinolinecarbonitrile, 4-(2,4-dichloro-5-methoxyanilino)-7-(5-{[4-(1-pyrrolidinyl)-1-piperidinyl]methyl}-2-furyl)-3-quinolinecarbonitrile, 4-(2,4-dichloro-5-methoxyanilino)-7-(5-{[(4-pyridinylmethyl)-amino]methyl}-2-furyl)-3-quinolinecarbonitrile, 4-(2,4-dichloro-5-methoxy-anilino)-7-[5-({[2-(1H-imidazol-4-yl)ethyl]amino}methyl)-2-furyl-3-quinolinecarbonitrile, 4-(2,4-dichloro-5-methoxyanilino)-7-(5-{[(2-hydroxyethyl)-amino]methyl}-2-furyl)-3-quinoline-carbonitrile, 4-(2,4-dichloro-5-methoxyanilino)-7-[5-({[3-(4-morpholinyl)propyl]amino}methyl)-2-furyl]-3-quinolinecarbonitrile, 4-(2,4-dichloro-5-methoxyanilino)-7-[5-(1-piperidinyl)methyl)-2-furyl]-3-quinolinecarbonitrile, 4-(2,4-dichloro-5-methoxy-anilino)-7-{5-[(4-ethyl-1-piperazinyl)methyl]-2-furyl}-3-quinolinecarbonitrile, 4-(2,4-dichloro-5-methoxyanilino)-7-(5-{[4-(1-pyrrolidinyl)-1-piperidinyl]methyl}-2-thienyl)-3-quinolinecarbonitrile, 4-(2,4-dichloro-5-methoxy-anilino)-7-{5-[(4-ethyl-1-piperazinyl)-methyl]-2-thienyl}-3-quinolinecarbonitrile, 4-(2,4-dichloro-5-methoxyanilino)-7-[5-({[2-(dimethylamino)ethyl]amino}methyl)-3-thienyl]-3-quinolinecarbonitrile, 4-(2,4-dichloro-5-methoxyanilino)-7-(5-[(4-pyridinylmethyl)-amino]methyl}-3-thienyl)-3-quinolinecarbonitrile, 4-(2,4-dichloro-5-methoxyanilino)-7-[5-({[2-(1H-imidazol-4-yl)ethyl]amino}methyl)-3-thienyl]-3-quinolinecarbonitrile, 4-(2,4-dichloro-5-methoxyanilino)-7-{5-[(4-hydroxy-1-piperidinyl)methyl]-2-pyridinyl}-3-quinolinecarbonitrile, 4-(2,4-dichloro-5-methoxyanilino)-7-[5-({[2-(dimethylamino)ethyl]amino}methyl)-2-pyridinyl]-3-quinolinecarbonitrile, 4-(2,4-dichloro-5-methoxyanilino)-7-(5-{[4-(1-pyrrolidinyl)-1-piperidinyl]methyl}-2-pyridinyl)-3-quinolinecarbonitrile, 4-(2,4-dichloro-5-methoxyanilino)-7-(5-{[(2-hydroxyethyl)-amino]methyl}-2-pyridinyl)-3-quinolinecarbonitrile, 4-(2,4-dichloro-5-methoxyanilino)-7-[5-({[3-(4-morpholinyl)-propyl]amino}methyl)-2-pyridinyl]-3-quinolinecarbonitrile, 4-(2,4-dichloro-5-methoxyanilino)-7-[(5-{[4-(2-hydroxyethyl)-1-piperazinyl]methyl)-2-pyridinyl]-3-quinolinecarbonitrile, 7-{4-[(4-hydroxy-1-piperidinyl)methyl]phenyl}-4-(3,4,5-trimethoxyanilino)-3-quinolinecarbonitrile, 7-[4-(4-morpholinylmethyl)phenyl]-4-(3,4,5-trimethoxyanilino)-3-quinolinecarbonitrile, 7-(4-{[(2-hydroxyethyl)amino]-methyl}phenyl)-4-(3,4,5-trimethoxyanilino)-3-quinolinecarbonitrile, 7-{4-[(4-methyl-1-piperazinyl)methyl]phenyl}-4-(3,4,5-trimethoxyanilino)-3-quinolinecarbonitrile, 7-[4-(1-piperidinyl)methyl)phenyl]-4-(3,4,5-trimethoxyanilino)-3-quinolinecarbonitrile, 7-{4-[(4-ethyl-1-piperazinyl)methyl]phenyl}-4-(3,4,5-trimethoxyanilino)-3-quinolinecarbonitrile, 7-{3-[(4-hydroxy-1-piperidinyl)methyl]phenyl}-4-(3,4,5-trimethoxyanilino)-3-quinolinecarbonitrile, 7-[3-({[2-(dimethylamino)ethyl]amino}methyl)phenyl]-4-(3,4,5-trimethoxyanilino)-3-quinolinecarbonitrile, 7-(3-[4-(1-pyrrolidinyl)-1-piperidinyl]-methyl}phenyl)-4-(3,4,5-trimethoxyanilino)-3-quinolinecarbonitrile, 7-(3-{[(4-pyridinylmethyl)amino]methyl}phenyl)-4-(3,4,5-trimethoxyanilino)-3-quinolinecarbo-nitrile, 7-[3-({[2-(1H-imidazol-4-yl)ethyl]amino}methyl)phenyl]-4-(3,4,5-trimethoxyanilino)-3-quinolinecarbonitrile, 7-[3-(4-morpholinylmethyl)phenyl]-4-(3,4,5-trimethoxy-anilino)-3-quinolinecarbonitrile, 7-(3-{[(2-hydroxyethyl)amino]methyl}phenyl)-4-(3,4,5-trimethoxyanilino-3-quinolinecarbonitrile, 7-{3-[(4-methyl-1-piperazinyl)methyl]-phenyl}-4-(3,4,5-trimethoxyanilino)-3-quinolinecarbonitrile, 7-[3-({[3-(4-morpholinyl)propyl]amino}methyl)phenyl]-4-(3,4,5-trimethoxyanilino)-3-quinoline-carbonitrile, 7-[3-(1-piperidinyl)methyl)phenyl]-4-(3,4,5-trimethoxyanilino)-3-quinoline-carbonitrile, 7-((3-{[4-(2-hydroxyethyl)-1-piperazinyl]methyl}phenyl)-4-(3,4,5-trimethoxyanilino)-3-quinolinecarbonitrile, 7-(5-{[4-(1-pyrrolidinyl)-1-piperidinyl]-methyl}-2-furyl)-4-(3,4,5-trimethoxyanilino)-3-quinolinecarbonitrile, 7-{-[(4-hydroxy-1-piperidinyl)methyl]-2-thienyl}-4-(3,4,5-trimethoxyanilino)-3-quinolinecarbonitrile, 7-[5-({[2-(1H-imidazol-4-yl)ethyl]amino}methyl)-2-thienyl]-4-(3,4,5-trimethoxyanilino)-3-quinolinecarbonitrile, 7-{5-[(4-methyl-1-piperazinyl)methyl]-2-thienyl}-4-(3,4,5-trimethoxyanilino)-3-quinolinecarbonitrile, 7-{5-[(4-ethyl-1-piperazinyl)methyl]-2-thienyl}-4-(3,4,5-trimethoxyanilino)-3-quinolinecarbonitrile, 7-(5-{[4-(2-hydroxyethyl)-1-piperazinyl]methyl}-2-thienyl)-4-(3,4,5-trimethoxyanilino)-3-quinolinecarbonitrile, 7-(5-{[(4-pyridinylmethyl)amino]methyl}-3-thienyl)-4-(3,4,5-trimethoxyanilino)-3-quinolinecarbonitrile, 7-[5-(4-morpholinylmethyl)-3-thienyl]-4-(3,4,5-trimethoxy-anilino)-3-quinolinecarbonitrile, 7-(5-{[(2-hydroxyethyl)amino]methyl}-3-thienyl)-4-(3,4,5-trimethoxyanilino)-3-quinolinecarbonitrile, 7-{5-[(4-methyl-1-piperazinyl)-methyl]-3-thienyl}-4-(3,4,5-trimethoxyanilino)-3-quinolinecarbonitrile, 7-{5-[(4-hydroxy-1-piperidinyl)methyl]-2-pyridinyl}-4-(3,4,5-trimethoxyanilino)-3-quinolinecarbonitrile, 7-(5-{[4-(1-pyrrolidinyl)-1-piperidinyl]methyl}-2-pyridinyl)-4-(3,4,5-trimethoxyanilino)-3-quinolinecarbonitrile, 7-(5-{[(2-hydroxyethyl)amino]methyl}-2-pyridinyl)-4-(3,4,5-trimethoxyanilino)-3-quinolinecarbonitrile, 7-[5-({[3-(4-morpholinyl)propyl]amino}-methyl)-2-pyridinyl)-4-(3,4,5-trimethoxyanilino)-3-quinolinecarbonitrile, 7-[(5-{[4-(2-hydroxyethyl)-1-piperazinyl]methyl)-2-pyridinyl]-4-(3,4,5-trimethoxyanilino)-3-quinolinecarbonitrile, BMS-354825, PP1, PP2, AZD-0530, AP-23464 and pharmaceutically acceptable salts thereof. The structure of bosutinib is

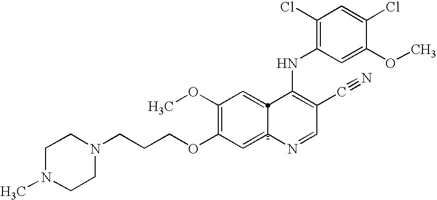

The structure of neratinib is

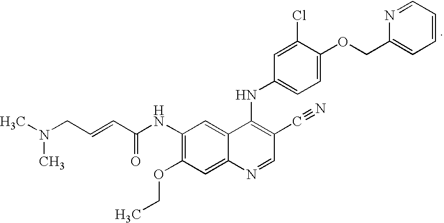

Aromatase is an enzyme that converts androgens to estrone. Estrone can subsequently be converted to estradiol, which has been linked to increased growth or proliferation of estrogen receptor positive carcinoma. As used in accordance with this invention, the term “aromatase inhibitor” means compounds or substances that inhibit the activity of the enzyme aromatase. Use of aromatase inhibitors in chemotherapy is employed in the invention typically to reduce the levels of circulating estradiol, to ultimately inhibit the growth of neoplasms that are estrogen receptor positive. Suitable examples of steroidal aromatase inhibitors include but are not limited to exemestane, formestane, and atamestane, and the like. Suitable examples of non-steroidal aromatase inhibitors include but are not limited to fadrozole, letrozole, vorozole, anastrozole, tamoxifen and YM511, as described by Susaki et al, in J. Steroid Biochem Molec Biol, 58:89-194, 1996. When used with an Src inhibitor, including bosutinib or neratinib, any of exemestane, tamoxifen or letrozole are preferred aromatase inhibitor in the combination of the invention. Exemestane, 10,13-dimethyl-6-methylidene-7,8,9,10,11,12,13,14,15,16-decahydrocyclopenta[a]phenanthrene-3,17-dione or analogs thereof are usefully employed in combination with the Src inhibitor. The structure of the steroidal aromatase inhibitor exemestane is disclosed in the publication of David Thurston, Chemistry and Pharmacology of Anti-cancer drugs, CRC Press: Boca Raton, p. 139 (2006). It has a different structure than other aromatase inhibitors:

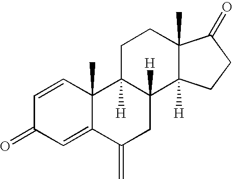

Letrozole, 4,4′-(1H-1,2,4-triazol-1-ylmethylene)bis-benzonitrile or 4,4′-(1H-1,2,4-triazol-1-ylmethylene)dibenzonitrile, is also usefully employed in the combination of the invention. Letrozole has a structure:

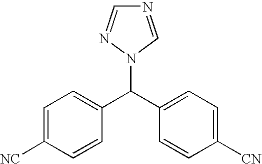

Tamoxifen, (Z)-2-[4-(1,2-diphenylbut-1-enyl)phenoxy]-N,N-dimethyl-ethanamine, is the trans-isomer of a triphenylethylene derivative of Taxol and is active in the treatment of breast cancer, an oestrogen antagonist. The structure of tamoxifen is:

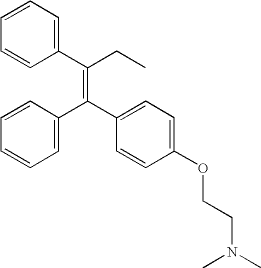

According to one embodiment, analogs of aromatase inhibitors and their corresponding pharmaceutically acceptable salts are usefully employed in combination with Src inhibitors in the pharmaceutical composition of the invention. One suitable example is 4-hydroxy tamoxifen.

Pharmaceutically acceptable salts of Src inhibitors and exemestane inhibitors may be formed from organic and inorganic acids. For example salts may be formed from when a compound of this invention contains a basic functional group. Other suitable examples of pharmaceutically acceptable salts include, but are not limited, to salts of fatty acids. “Pharmaceutically acceptable salts” include but are not limited to: alkali metal or alkaline earth metal salts such as sodium, potassium, lithium, calcium, or magnesium; N-tetraalkylammonium salts such as N-tetrabutylammonium salts; acetate; amsonate (4,4-diaminostilbene-2,2-disulfonate); ascorbate; benzenesulfonate; benzonate; bicarbonate; bisulfate; bitartrate; borate; bromide; butyrate; calcium edetate; camsylate; carbonate; chloride; citrate; clavulariate; dihydrochloride; edetate; edisylate; estolate; esylate; formate; fumarate; gentisinate; glucaronate; gluceptate; gluconate; glutamate; glycollylarsanilate; hexafluorophosphate; hexylresorcinate; hydrabamine; hydrobromide; hydrochloride; hydroxynaphthoate; iodide; isonicotinate; isothionate; lactate; lactobionate; laurate; linoleate; linolenate; malate; maleate; malonate; mandelate; mesylate; methylbromide; methylnitrate; methylsulfate; mucate; myristate; napsylate; nitrate; N-methylglucamine ammonium salt; 3-hydroxy-2-naphthoate; oleate; oxalate; palmitate; pamoate (4,4′-methylenebis-3-hydroxy-2-naphthoate, or embonate); pantothenate; phthalate; phosphate/diphosphate; picrate; polygalacturonate; propionate; p-toluenesulfonate; salicylate; stearate; subacetate; saccharate; succinate; sulfate; sulfosaliculate; suramate; tannate; tartrate; teoclate; tosylate; triethiodide; and valerate salts. The compounds of the pharmaceutical combination can also be used in the form of esters, carbamates and other conventional ester forms, also referred to herein as prodrug forms, which when administered in such form, convert to the active moiety in-vivo. Exemplary ester forms of the compounds in the pharmaceutical combination of this invention include, but are not limited to, straight chain alkyl esters having from 1 to 6 carbon atoms or branched chain alkyl groups containing 1 to 6 carbon atoms, including methyl, ethyl, propyl, butyl, 2-methylpropyl and 1,1-dimethylethyl esters, cycloalkyl esters, alkylaryl esters, benzyl esters, and the like. The pharmaceutically acceptable combination of an Src inhibitor, exemestane and corresponding pharmaceutically acceptable salts or esters thereof include isomers either individually or as a mixture, such as enantiomers, diastereomers, and positional isomers.

According to one embodiment, the present invention provides a pharmaceutical composition comprising a therapeutically effective amount of a Src kinase inhibitor, or a pharmaceutically acceptable salt thereof, a therapeutically effective amount of an aromatase inhibitor, or a pharmaceutically acceptable salt thereof, and a pharmaceutically acceptable carrier, wherein the amounts of the Src kinase inhibitor and the aromatase inhibitor in the composition are such that the combined therapeutic effect of the two active ingredients is synergistic.

According to one embodiment, the Src kinase inhibitor is selected from bosutinib, dasatinib, neratinib, PP1, PP2, AP23464 and PD166326 or a pharmaceutically acceptable salt thereof, and the aromatase inhibitor selected from anastrozole, letrozole, vorazole, tamoxifen and exemestane or a pharmaceutically acceptable salt thereof, and a pharmaceutically acceptable carrier.

According to one embodiment, the pharmaceutically acceptable combination is bosutinib and exemestane or pharmaceutically acceptable salts thereof.

According to one embodiment, the pharmaceutically acceptable combination is bosutinib and leterozole or pharmaceutically acceptable salts thereof.

According to one embodiment, the pharmaceutically acceptable combination is bosutinib and tamoxifen or pharmaceutically acceptable salts thereof.

According to one embodiment, the pharmaceutically acceptable combination is neratinib and exemestane or pharmaceutically acceptable salts thereof.

According to one embodiment, the pharmaceutically acceptable combination is neratinib and tamoxifen or pharmaceutically acceptable salts thereof.

According to one embodiment, the pharmaceutically acceptable combination is neratinib and letrozole or pharmaceutically acceptable salts thereof.

Based on the results of standard pharmacological test procedures described herein, the combination of a therapeutically effective amount of a Src kinase inhibitor, or a pharmaceutically acceptable salt thereof, a therapeutically effective amount of an aromatase inhibitor, or a pharmaceutically acceptable salt thereof, and a pharmaceutically acceptable carrier, is useful for treating, inhibiting or controlling the growth of cancerous tumor cells and associated diseases in a mammal in need thereof. In the case of cancer treatment, it is believed that many neoplasias such as leukemia, lung cancer, colon cancer, thyroid cancer, ovarian cancer, renal cancer, prostate cancer and breast cancers may be treated by effectively administering effective amounts of the pharmaceutical combination of the invention. Suitable examples of cancers for treatment using methods provided herein include carcinoma, sarcoma, lymphoma, or leukemia. The term “carcinoma” refers to a benign or malignant epithelial tumor and includes, but is not limited to, breast carcinoma, prostate carcinoma, non-small lung carcinoma, colon carcinoma, melanoma carcinoma, ovarian carcinoma, or renal carcinoma. A preferred subject or mammalian host benefiting from treatment using one or more compounds of the invention is a human. Use of a combination of the Src inhibitor and the aromatase inhibitor also provides for the use of combinations in which the Src inhibitor and/or aromatase inhibitor is used at a subtherapeutically effective dosage. A subtherapeutically effective dosage refers to a dose lower than the amount that is effective when the drug is delivered alone (monotherapy). Although less desirable, it is possible that one of the active agents may be used in a supratherapeutic amount, i.e., at a higher dosage in the combination than when used alone. In this embodiment, the other active agent(s) may be used in a therapeutic or subtherapeutic amount.

In one embodiment, this combination therapy comprises delivering a combination of bosutinib and exemestane. According to a separate embodiment, the combination therapy comprises delivering a combination of neratinib and exemestane.

In one embodiment, this combination therapy comprises delivering a combination of bosutinib and tamoxifen. According to a separate embodiment, the combination therapy comprises delivering a combination of neratinib and tamoxifen.

In one embodiment, this combination therapy comprises delivering a combination of bosutinib and letrozole. According to a separate embodiment, the combination therapy comprises delivering a combination of neratinib and letrozole.

According to one embodiment, the pharmaceutical combination of a Src inhibitor and an aromatase inhibitor is useful for treating cancers, inhibiting angiogenesis and metastatic cancers. Both the Src inhibitor, such as bosutinib or neratinib for example, and exemestane exhibit anti-tumor activity when administered as single agents to breast cancer subjects.

In one embodiment, the pharmaceutical composition comprising a combination of bosutinib and exemestane exhibits synergistic effects in post-menopausal women with locally advanced or metastatic ER+/PgR+/Her2-breast cancer. In a separate embodiment, the pharmaceutical composition comprising a combination of neratinib and exemestane exhibits synergistic effects in treating an Erb-2 associated or overexpressing neoplasms and post-menopausal women with locally advanced or metastatic ER+/PgR+/Her2-breast cancer. For example, the breast cancer can be an Erb-2 positive metastatic breast cancer or a locally advanced breast cancer. In another embodiment, the neoplasm is an Erb-2 positive solid tumor. In one embodiment, a regimen as provided herein is used for treating a neoplasm characterized by an erB-2 (HER-2) overexpressing neoplasm. In another embodiment, a regimen as provided herein is used for treating a neoplasm characterized by overexpression of an erB-1 overexpressing neoplasm. In still another embodiment, a regimen as described herein is used for treating a breast cancer. In certain embodiments, the breast cancer may be an erB-2-overexpressing metastatic or locally advanced breast cancer.

In one embodiment, the pharmaceutical composition comprising a combination of bosutinib and letrozole exhibits synergistic effects in post-menopausal women with locally advanced or metastatic ER+/PgR+/Her2-breast cancer. In a separate embodiment, the pharmaceutical composition comprising a combination of neratinib and letrozole exhibits synergistic effects in treating an Erb-2 associated or overexpressing neoplasms and post-menopausal women with locally advanced or metastatic ER+/PgR+/Her2-breast cancer. For example, the breast cancer can be an Erb-2 positive metastatic breast cancer or a locally advanced breast cancer. In another embodiment, the neoplasm is an Erb-2 positive solid tumor. In one embodiment, a regimen as provided herein is used for treating a neoplasm characterized by an erB-2 (HER-2) overexpressing neoplasm. In another embodiment, a regimen as provided herein is used for treating a neoplasm characterized by overexpression of an erB-1 overexpressing neoplasm. In still another embodiment, a regimen as described herein is used for treating a breast cancer. In certain embodiments, the breast cancer may be an erB-2-overexpressing metastatic or locally advanced breast cancer.

In one embodiment, the pharmaceutical composition comprising a combination of bosutinib and tamoxifen exhibits a desired therapeutic and cooperative effects in post-menopausal women with locally advanced or metastatic ER+/PgR+/Her2-breast cancer. In a separate embodiment, the pharmaceutical composition comprising a combination of neratinib and tamoxifen exhibits desired therapeutic effects in treating an Erb-2 associated or overexpressing neoplasms and post-menopausal women with locally advanced or metastatic ER+/PgR+/Her2-breast cancer. For example, the breast cancer can be an Erb-2 positive metastatic breast cancer or a locally advanced breast cancer. In another embodiment, the neoplasm is an Erb-2 positive solid tumor. In one embodiment, a regimen as provided herein is used for treating a neoplasm characterized by an erB-2 (HER-2) overexpressing neoplasm. In another embodiment, a regimen as provided herein is used for treating a neoplasm characterized by overexpression of an erB-1 overexpressing neoplasm. In still another embodiment, a regimen as described herein is used for treating a breast cancer. In certain embodiments, the breast cancer may be an erB-2-overexpressing metastatic or locally advanced breast cancer.

According to another embodiment, bosutinib administered in combination with exemestane versus exemestane alone is more efficacious as a second line therapy in post-menopausal women with locally advanced or metastatic ER+/PgR+/Her2-breast cancer. The amounts of the Src kinase inhibitor and exemestane in the composition are such that the combined therapeutic effect of the two active ingredients is synergistic.

According to another embodiment, bosutinib administered in combination with letrozole versus letrozole alone is more efficacious as a second line therapy in post-menopausal women with locally advanced or metastatic ER+/PgR+/Her2-breast cancer. The amounts of the Src kinase inhibitor and letrozole in the composition are such that the combined therapeutic effect of the two active ingredients is synergistic.

According to another embodiment, bosutinib administered in combination with tamoxifen versus tamoxifen alone is more efficacious as a second line therapy in post-menopausal women with locally advanced or metastatic ER+/PgR+/Her2-breast cancer.

According to one embodiment, the pharmaceutical combination of bosutinib and exemestane is particularly effective in human subjects with ER+ breast cancer (HER2-), since most subjects with ER+ breast cancer do not require chemotherapy.

According to one embodiment, the pharmaceutical combination of bosutinib and letrozole is particularly effective in human subjects with ER+ breast cancer (HER2-), since most subjects with ER+ breast cancer do not require chemotherapy.

According to one embodiment, the pharmaceutical combination of bosutinib and tamoxifen is particularly effective in human subjects with ER+ breast cancer (HER2-), since most subjects with ER+ breast cancer do not require chemotherapy.

According to another embodiment, the pharmaceutical comprising the combination of the Src inhibitor and the aromatase inhibitor can be used in a further combination with one or more anti-cancer agents. The term “anticancer” agent includes any known agent that is useful for the treatment of cancer including, but not limited to for example, 17α-ethinylestradiol, diethylstilbestrol, testosterone, prednisone, fluoxymesterone, dromostanolone propionate, testolactone, megestrolacetate, methylprednisolone, methyl-testosterone, prednisolone, triamcinolone, chlorotrianisene, hydroxyprogesterone, aminoglutethimide, estramustine, medroxyprogesteroneacetate, leuprolide, flutamide, toremifene, Zoladex; matrix metalloproteinase inhibitors; VEGF inhibitors, such as anti-VEGF antibodies (Avastin) and small molecules such as ZD6474, AZD-2171, and SU6668; Vatalanib, BAY-43-9006, SU11248, CP-547632, and CEP-7055; Her 1 and Her 2 inhibitors including anti-Her2 antibodies (Herceptin); EGFR inhibitors including gefitinib, erlotinib, ABX-EGF, EMD72000, 11F8, and cetuximab; Eg5 inhibitors, such as SB-715992, SB-743921, and MKI-833; pan Her inhibitors, such as canertinib, EKB-569, CI-1033, AEE-788, XL-647, mAb 2C4, and GW-572016; Casodex®, Bcr-AbI.inhibitors (GLEEVAC), (bicalutamide, Astra Zeneca); MEK-1 kinase inhibitors, MAPK kinase inhibitors, PI3 kinase inhibitors; Met inhibitors, aurora kinase inhibitors, PDGF inhibitors, such as imatinib; anti-angiogenic and antivascular agents which, by interrupting blood flow to solid tumors, render cancer cells quiescent by depriving them of nutrition; castration, which renders androgen dependent carcinomas non-proliferative; IGF1 R inhibitors such as those disclosed in U.S. Ser. No. 2004/44203A1, inhibitors of non-receptor and receptor tyrosine kinases; inhibitors of integrin signaling; tubulin acting agents such as vinblastine, vincristine, vinorelbine, vinflunine, paclitaxel, docetaxel, 7-O-methylthiomethylpaclitaxel, 4-desacetyl-4-methylcarbonatepaclitaxel, 3′-tert-butyl-3′-N-tert-butyloxycarbonyl-4-deacetyl-3′-dephenyl-3′-N-debenzoyl-4-O-methoxy-carbonylpaclitaxel, C-4 methyl carbonate paclitaxel, epothilone A, epothilone B, epothilone C, epothilone D, desoxyepothilone A, desoxyepothilone B, ixabepilone, [1S-[1R*,3R*(E),7R*,10S*, 11R*,12R*,16S*]]3-[2-[2-(aminomethyl)-4-thiazolyl]-1-methylethenyl]-7,11-dihydroxy-8,8,10,12,16-pentamethyl-4-17-dioxabicyclo[14.1.0]-heptadecane-5,9-dione, and derivatives thereof; CDK inhibitors, antiproliferative cell cycle inhibitors, epidophyllotoxin, etoposide, VM-26; antineoplastic enzymes, e.g., topoisomerase I inhibitors, camptothecin, topotecan, SN-38; procarbazine; mitoxantrone; platinum coordination complexes such as cisplatin, carboplatin and oxaliplatin; biological response modifiers; growth inhibitors; antihormonal therapeutic agents; leucovorin; tegafur; antimetabolites such as purine antagonists (e.g. 6-thioguanine and 6-mercaptopurine; glutamine antagonists, e.g. DON (AT-125; d-oxo-norleucine); ribonucleotide reductase inhibitors; mTOR inhibitors; and haematopoietic growth factors. In a separate embodiment the combination of the invention is used with radiation and other radiation based treatments.

In general, the combination of the Src inhibitor and aromatase inhibitor can be administered as pharmaceutical compositions by any method known in the art for administering therapeutic drugs including oral, buccal, topical, systemic (e.g., transdermal, intranasal, or by suppository), or parenteral (e.g., intramuscular, subcutaneous, or intravenous injection). Compositions can take the form of tablets, pills, capsules, semisolids, powders, sustained release formulations, solutions, suspensions, emulsions, syrups, elixirs, aerosols, or any other appropriate compositions; and comprise at least one compound of this invention in combination with at least one pharmaceutically acceptable excipient. Suitable excipients are well known to persons of ordinary skill in the art, and they, and the methods of formulating the compositions, can be found in such standard references as Alfonso A R: Remington's Pharmaceutical Sciences, 17th ed., Mack Publishing Company, Easton Pa., 1985. Suitable liquid carriers, especially for injectable solutions, include water, aqueous saline solution, aqueous dextrose solution, and glycols. In some embodiments of the present invention, the pharmaceutical composition suitable for use in the practice of this invention will be administered either singly or in combination with at least one other compound of this invention. The combination for use in the practice of the present invention can also be administered with at least one other conventional therapeutic agent for the disease being treated. Pharmaceutical compositions of the invention may preferably be orally administered, for example, with an inert diluent or with an assimilable edible carrier, or they may be enclosed in hard or soft shell gelatin capsules, or they may be compressed into tablets or they may be incorporated directly with the food of the diet. For oral therapeutic administration, these active compounds may be incorporated with excipients and used in the form of ingestible tablets, buccal tablets, troches, capsules, elixirs, suspensions, syrups, wafers and the like. Such compositions and preparations should contain at least 0.1% of active compound. The percentage of the compositions and preparations may, of course, be varied and may conveniently be between about 2% to about 60% of the weight of the unit. The amount of active compound in such therapeutically useful compositions is such that a suitable dosage will be obtained. Preferred compositions or preparations according to the present invention are prepared so that an oral dosage unit form contains between 10 and 1000 mg of active compound.

The Src inhibitor can be administered, e.g., orally, at a dose range of about 0.01 to 100 mg/kg. In one embodiment, the Src inhibitor is administered at a dose range of about 0.1 to about 90 mg/kg. In another embodiment, the Src inhibitor is administered at a dose range of about 1 to about 80 mg/kg. In a further embodiment, the Src inhibitor is administered at a dose range of about 10 to about 70 mg/kg. In yet another embodiment, the Src inhibitor is administered at a dose range of about 15 to about 60 mg/kg. In still a further embodiment, the Src inhibitor is administered at a dose range of about 20 to about 240 mg per day, at least about 40 mg, at least about 120 mg, or at least about 160 mg, or at least about 200 mg, on the days in the cycle on which it is administered. One of skill in the art could routinely perform empirical activity tests to determine the bioactivity of the compound in bioassays and thus determine what dosage to administer when the compound is delivered by another route. In one embodiment, the oral dosage of the Src inhibitor is at least about 600 mg/week. In another embodiment, the oral dosage of the Src inhibitor is about 800 mg/week to at least to about 1700 mg/week. In another embodiment, the oral dosage of the Src inhibitor is about 840 mg/week to about 1680 mg/week. Precise dosages are determined by the administering physician based on experience with the individual subject to be treated. Other dosage regimens and variations are foreseeable, and are determined through physician guidance.

Aromatase inhibitors may be used according to the currently approved/recommended dose of exemestane for monotherapy of breast cancer, i.e., an amount equivalent to 100 mg/kg² administered orally twice daily (equivalent to 2500 mg/kg² total daily dose) for 14 days followed by a 7-day rest period given as 3-week cycles, for as long as needed. Typically the mean duration of treatment is 3 to 6 three-week cycles. In another embodiment, the doses of exemestane may be reduced for use in the combination therapy of the present invention. Alternatively, high doses of exemestane may be used for a period of one to multiple days, with reduced doses being delivered on certain days within a cycle. For example, a daily starting oral dose may be in the range of, e.g., 1 mg to 3000 mg, 100 mg to 4000 mg, 1500 mg to 2000 mg, 2000 mg to about 3600 mg, or about 2400 mg to about 3600 mg per day, on the days in the cycle on which it is administered. In another embodiment, the combination of the invention permits lower daily doses (subtherapeutic) of exemestane to be used, thus minimizing the risk of dose-limiting side effects. In one embodiment, the daily dose of an aromatase inhibitor, including exemestane, letrozole or tamoxifen is 25 mg to 2000 mg, 900 to 1800 mg, or about 1250 mg to 1450 mg/day. Precise dosages are determined by the administering physician based on experience with the individual subject to be treated. Other dosage regimens and variations are foreseeable, and are determined through physician guidance. In a separate embodiment, the aromatase inhibitor is administered by i.v. infusion or orally, preferably in the form of tablets or capsules. In a separate embodiment, subtherapeutically effective amounts of the Src inhibitor and exemestane may be used to achieve a therapeutic effect when administered in combination. In one embodiment, the Src inhibitor is provided at dosages of 5 to 50% lower when provided along with the aromatase inhibitor. In another embodiment, the Src inhibitor is provided at dosages of 10 to 25% lower when provided along with the exemestane. In a further embodiment, the Src inhibitor is provided at dosages of 15 to 20% lower when provided along with the aromatase inhibitor. In one embodiment, a resulting Src inhibitor dosage is about 8 to 40 mg. Subtherapeutically effective amounts of the Src inhibitor and the aromatase inhibitor are expected to reduce the side-effects of treatment.

According to one embodiment, the pharmaceutical combination of the Src inhibitor and the aromatase inhibitor are administered simultaneously to a subject. In a separate embodiment, the Src inhibitor is administered to a subject prior to administering the aromatase inhibitor to the subject. In another embodiment, the aromatase inhibitor is administered to a subject prior to administering the Src inhibitor to the subject. In addition, the aromatase inhibitor/pr the Src inhibitor may also be administered after completion of chemotherapy as maintenance therapy.

According to one embodiment, the pharmaceutical combination of the Src inhibitor and the aromatase inhibitor are administered orally to a subject. The compounds of this invention can be administered orally. The amount of a compound of the present invention in the composition can vary widely depending on the type of composition, size of a unit dosage, kind of excipients, and other factors well known to those of ordinary skill in the art. In general, the final composition can comprise from, for example, 0.000001 percent by weight (% wt.) to 10% wt. of the compound of formula I, preferably 0.00001% wt. to 1% wt., with the remainder being the excipient or excipients. In a separate embodiment, the pharmaceutical combination of the Src inhibitor and the aromatase inhibitor is administered intravenously to the subject.

Clinical regimens described herein may also include the administration of other active agents which are not anti-neoplastics, but which ameliorate the symptoms of the neoplastic disease and/or therapy. In a still further embodiment, the combination may include an anti-diarrheal. One of skill in the art would readily be able to select a suitable antidiarrheal for use herein including, without limitation, loperamide or diphenoxylate hydrochloride and atropine sulfate. Alternatively, the anti-diarrheal may be administered to the patient prior to or subsequent to treatment with the combination of the invention. In a further embodiment, the combination further contains an antiemetic agent. Examples of antiemetic agents include, without limitation, metoclopramide, Dolasetron, Granisetron, Ondansetron, Tropisetron, and Palonosetron, among others. Alternatively, the antiemetic may be administered to the patient prior to or subsequent to treatment with the combination of the invention. In yet a further embodiment, the combination also contains an antihistamine. Examples of antihistamines include, without limitation, Cyclizine, Diphenhydramine, Dimenhydrinate, Meclizine, Promethazine or Hydroxyzine, among others. Alternatively, the antihistamine may be administered to the patient prior to or subsequent to treatment with the combination of the invention. In yet another embodiment, the combination may include a growth factor to prevent and/or treat neutropenia. Such growth factors may readily be selected by those having skill in the art according to practice guidelines from the American Society of Clinical Oncology (ASCO; 2006). Alternatively, the growth factor may be administered to the patient prior to or subsequent to treatment with the combination of the invention. In still another embodiment, the regimen may be used in combination with other anti-neoplastic agents. As is typical with oncology treatments, dosage regimens are closely monitored by the treating physician, based on numerous factors including the severity of the disease, response to the disease, any treatment related toxicities, age, and health of the patient. Dosage regimens are expected to vary according to the route of administration. The dosages and schedules described hereinbefore may be varied according to the particular disease state and the overall condition of the patient. For example, it may be necessary or desirable to reduce the above-mentioned doses of the components of the combination treatment in order to reduce toxicity. Dosages and schedules may also vary if, in addition to a combination of an Src inhibitor and exemestane, one or more additional chemotherapeutic agents are used. Scheduling may be determined by the practitioner whom is treating any particular patient using his professional skill and knowledge.

Aqueous suspensions of the pharmaceutical combination invention can contain the components in admixture with excipients suitable for the manufacture of aqueous suspensions. Such excipients can include a suspending agent, such as sodium carboxymethylcellulose, methylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents, and one or more sweetening agents, such as sucrose, aspartame or saccharin. Formulations can be adjusted for osmolarity.

Oil suspensions can be formulated by suspending components of the pharmaceutical composition in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations of the invention can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent. Under ordinary conditions of storage and use, these preparations contain a preservative to prevent the growth or microorganisms.

Formulations suitable for parenteral administration, such as, for example, by intraarticular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Among the acceptable vehicles and solvents that can be employed are water and Ringer's solution, an isotonic sodium chloride. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables. These solutions are sterile and generally free of undesirable matter. Where the compounds are sufficiently soluble they can be dissolved directly in normal saline with or without the use of suitable organic solvents, such as propylene glycol or polyethylene glycol. Dispersions of the finely divided compounds can be made-up in aqueous starch or sodium carboxymethyl cellulose solution, or in suitable oil, such as arachis oil. These formulations can be sterilized by conventional, well-known sterilization techniques. The formulations can contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the active compounds of the pharmaceutical combination in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol. The formulations of the combination can be presented in unit-dose or multi-dose sealed containers, such as ampules and vials. Injection solutions and suspensions can be prepared from sterile powders, granules, and tablets of the kind previously described.

The compounds of the present invention can also be administered in the form of suppositories for rectal administration of the drug. These formulations can be prepared by mixing the drug with a suitable non-irritating excipient, which is solid at ordinary temperatures but liquid at the rectal temperatures and will therefore melt in the rectum to release the drug. Such materials are cocoa butter and polyethylene glycols.

The combination of the present invention can also be administered by intranasal, intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995).

The combination of the invention can be delivered transdermally, by a topical route, formulated as applicator sticks, solutions, suspensions, emulsions, gels, creams, ointments, pastes, jellies, paints, powders, and aerosols.

Encapsulating materials can also be employed with the component compounds of the combination of the present invention and the term “composition” can include the active ingredient in combination with an encapsulating material as a formulation, with or without other carriers. For example, the compounds of the present invention can also be delivered as microspheres for slow release in the body. In one embodiment, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao, Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months. Cachets can also be used in the delivery of the compounds of the present invention, e.g., anti-atherosclerotic medicaments.

In another embodiment, the pharmaceutical composition of the present invention can be delivered by the use of liposomes which fuse with the cellular membrane or are endocytosed, i.e., by employing ligands attached to the liposome, or attached directly to the oligonucleotide, that bind to surface membrane protein receptors of the cell resulting in endocytosis. By using liposomes, particularly where the liposome surface carries ligands specific for target cells, or are otherwise preferentially directed to a specific organ, one can focus the delivery of the compound into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). In other cases, the preferred preparation can be a lyophilized powder which may contain, for example, any or all of the following: 1 mM-50 mM histidine, 0.1%-2% sucrose, 2%-7% mannitol, at a pH range of 4.5 to 5.5, that is combined with buffer prior to use.

Also included is a product or pharmaceutical pack containing a course of an anti-neoplastic treatment for one individual mammal comprising one or more container(s) having one, one to four, or more unit(s) of an Src inhibitor in unit dosage form and one, one to four, or more unit(s) of the aromatase inhibitor and optionally, another active agent. The combinations may be in the form of a kit of parts. For the Src inhibitor and/or the aromatase inhibitor, it is desired each compound of the combination of compounds is in the form of a unit dose. The term “unit dose” or “unit dose form” as used herein describes a single dose form including, without limitation, tablets, caplets, capsules, powders in sachets or vials, saline infusion bags, as described above. In one embodiment a kit includes a first container with a suitable composition containing an Src inhibitor and a second container with a suitable composition containing the aromatase inhibitor. Accordingly, there is provided a kit for use in the treatment or prophylaxis of cancer. This kit includes comprising: a) an Src inhibitor together with a pharmaceutically-acceptable excipient or carrier, in a first unit dosage form; b) an aromatase inhibitor together with a pharmaceutically-acceptable excipient or carrier, in a second unit dosage form; and c) a container for containing said first and second dosage forms.

In one embodiment, the pharmaceutical pack comprises (a) at least one unit dose of exemestane a pharmaceutically acceptable salt thereof; and (b) at least one unit dose of bosutinib or a pharmaceutically acceptable salt thereof.

Unit dose forms contain from about 0.1 to about 300 mg of an Src inhibitor compound. In another embodiment, the unit dose form contains about 5 to about 800 mg of the Src inhibitor. In another embodiment, the unit dose form contains about 50 to about 300 mg of the Src inhibitor. In a further embodiment, the unit dose form contains about 75 to about 300 mg of the Src inhibitor. Currently, unit doses of the aromatase inhibitor are commercially available, for example as 150 mg or 500 mg tablets. However, other suitable unit doses may be prepared as desired or required. The invention therefore includes administering an Src inhibitor and the aromatase inhibitor to a subject for the treatment of a neoplasm. In one embodiment, the Src inhibitor is administered separately from the aromatase inhibitor. In a further embodiment, the Src inhibitor is administered prior to the aromatase inhibitor. In another embodiment, the Src inhibitor is administered subsequent to the aromatase inhibitor. In still another embodiment, the Src inhibitor and the aromatase inhibitor are administered simultaneously, but separately. In one embodiment, the Src inhibitor and the aromatase inhibitor are administered together as a combined preparation. In one embodiment, a product contains the Src inhibitor and the aromatase inhibitor as a combined preparation for simultaneous, separate or sequential use in treating a neoplasm in a mammal in need thereof. In one embodiment, the Src inhibitor is separately formulated from the aromatase inhibitor.

In one embodiment, a pharmaceutical pack contains a course of treatment of a neoplasm for one individual mammal, wherein the pack contains units of an Src inhibitor in unit dosage form and units of the aromatas inhibitor in unit dosage form. In another embodiment, a pharmaceutical pack contains a course of treatment of a neoplasm for one individual mammal, wherein the pack contains units of an Src inhibitor in unit dosage form and units of the aromatase inhibitor in unit dosage form.

In yet another embodiment, a pharmaceutical pack as described herein contains a course of treatment of metastatic breast cancer for one individual mammal. Administration of the individual components or a composition containing two or more of the individual components may employ any suitable route. Such routes may be selected from, e.g., oral, intravenous (i.v.), respiratory (e.g., nasal or intrabronchial), infusion, parenteral (aside from i.v., such as intralesional, intraperitoneal and subcutaneous injections), intraperitoneal, transdermal (including all administration across the surface of the body and the inner linings of bodily passages including epithelial and mucosal tissues), and vaginal (including intrauterine administration). Other routes of administration are also feasible and include, without limitation, liposome-mediated delivery, topical, nasal, sublingual, uretheral, intrathecal, ocular or otic delivery, implant, rectal, or intranasal. While the components may be delivered via the same route, a product or pack described herein may contain exemestane for delivery by a different route than that of an Src inhibitor, e.g., one or more of the components may be delivered orally, while the other is administered by another route.

In one embodiment, the Src inhibitor is prepared for oral delivery and the aromatase inhibitor is prepared for intravenous delivery. In one embodiment, the aromatase inhibitor is prepared for oral delivery and the Src inhibitor is prepared for intravenous delivery. Optionally, other active components may be delivered by the same or different routes as the Src inhibitor and/or the aromatase inhibitor. Other variations would be apparent to one skilled in the art.

In another embodiment, pharmaceutical packs contain a course of anti-neoplastic treatment for one individual mammal comprising a container having a unit of an Src inhibitor in unit dosage form, a containing having a unit of an aromatase inhibitor, and optionally, a container with another active agent. In some embodiments, the compositions are in packs in a form ready for administration. In other embodiments, the compositions are in concentrated form in packs, optionally with the diluent required to make a final solution for administration. In still other embodiments, the product contains a compound described herein in solid form and, optionally, a separate container with a suitable solvent or carrier. In still other embodiments, the above packs/kits include other components, e.g., instructions for dilution, mixing and/or administration of the product, other containers, syringes, needles, etc. Other such pack/kit components are readily apparent to one of skill in the art.

In still another embodiment, the compounds or components of the therapeutic regimen are administered once a week. In certain situations, dosing with the Src inhibitor may be delayed or discontinued for a brief period (e.g., 1, 2 or three weeks) during the course of treatment. Such a delay or discontinuation may occur once, or more, during the course of treatment. The effective amount is known to one of skill in the art; it will also be dependent upon the form of the Src inhibitor. One of skill in the art could routinely perform empirical activity tests to determine the bioactivity of the Src inhibitor in bioassays and thus determine a suitable dosage to administer.

The Src inhibitor and aromatase inhibitor combination or other optional compounds used in the combination and products described herein may be formulated in any suitable manner. However, the amounts of each compound in the unit dose can vary widely depending on the type of composition, regimen, size of a unit dosage, kind of excipients, and other factors well known to those of ordinary skill in the art. In one embodiment, the unit dose can contain, e.g., 0.000001 percent by weight (% w) to 10% w of either compound. In another embodiment the unit dose can contain about 0.00001% w to 1% w, with the remainder being the excipient or excipients.

For treatment purposes, the compositions or compounds disclosed herein can be administered to the subject in a single bolus delivery, via continuous delivery (e.g., continuous transdermal, mucosal, or intravenous delivery) over an extended time period, or in a repeated administration protocol (e.g., by an hourly, daily or weekly, repeated administration protocol). The pharmaceutical formulations of the present invention can be administered, for example, one or more times daily, 3 times per week, or weekly. In an exemplary embodiment of the present invention, the pharmaceutical formulations of the present invention are orally administered once or twice daily.

In an exemplary embodiment of the present invention, unit dosage forms of the either of the Src inhibitor or the aromatase inhibitor are prepared for standard administration regimens. In this way, the composition can be subdivided readily into smaller doses at the physicians direction. For example, unit dosages can be made up in packeted powders, vials or ampoules and preferably in capsule or tablet form. The active compound present in these unit dosage forms of the composition can be present in an amount of, for example, from about one gram to about fifteen grams or more, for single or multiple daily administration, according to the particular need of the patient. By initiating the treatment regimen with a minimal daily dose of about one gram, the blood levels of securin and the patients symptomatic relief analysis can be used to determine whether a larger or smaller dose is indicated. Effective administration of the compounds of this invention can be given at an oral dose of from, for example about 0.1 mg/kg/day to about 1,000 mg/kg/day. Preferably, administration of each active compound will be from about 10/mg/kg/day to about 600 mg/kg/day, more preferably from about 25 to about 750 mg/kg/day, and even more preferably from about 50 mg/kg/day to about 500 mg/kg/day.

Having described the invention, the invention is further illustrated by the following non-limiting examples.

### EXAMPLES

### Cell Proliferation-Assays and Materials

Cell lines were obtained from ATCC. Estrogen was obtained from SIGMA, 4-hydroxytamoxifen was obtained from SIGMA, Bosutinib was used for these studies. Charcoal-stripped serum and phenol red-free medium was obtained from Invitrogen. CellTiter Glo was obtained from Promega.

Cell cultures. Proliferation assays with cells in complete fetal calf serum were performed with medium containing phenol red. For all assays examining estrogen-dependence, cells were cultured in phenol red-free medium; DMEM/F12 (MCF7), RPMI1640 (MDA-MB-361, T47D) MEM (BT20, MDA-MB-231) McCoys5A modified (SKBR3), Leibowitz' L15 medium supplemented with 10 ng/mL insulin (MDA-MB-435 and MDA-MB-468) with 10% fetal bovine serum supplemented with glutamine and gentamicin. For proliferation assays, trypsinized cells were subjected to three cycles of centrifugation and resuspension in phenol red-free medium supplemented with charcoal stripped medium (5% final), glutamine and Pen/Strep. Cells were plated on day 0, with or without estrogen (10 nM final concentration). On day 1, 4-hydroxytamoxifen and SKI-606 or DMSO were added as a serial dilution. Cells were incubated for 7 days, after which CellTiter Glo (Promega) was added as recommended by the manufacturer. After mixing and two hours incubation on the bench, plates were read on an Envision reader. IC₅₀s were calculated using the LSW Data Analysis Plug-in for Excel. maximal concentration of 10 uM SKI-606 used in these assays, some toxicity was seen, e.g., BT20 and MDA-MB-361 cells. The 10 uM data point was dropped for these two lines since there was a clear plateau prior to the sharp drop in luminescence observed at 10 uM compound. The IC₅₀ was calculated using a floating minima corresponding to the lowest luminescence value at 5 uM.

Radis Procedure 2484, MCF-7 model was used for the measurement of in vitro estrogenic activity. MCF-7 cells were passaged twice a week in D-MEM/F-12 medium containing 10% (v/v) heat-inactivated fetal bovine serum, 1% (v/v) Penicillin-Streptomycin, and 2 mM glutaMax-1. The cells were plated with growth medium at 25,000/well into 96 well plates and incubated at 37° C. overnight.

The cells were infected for 2 hr at 37° C. with 50 μl/well of a 1:10 dilution of adenovirus 5-ERE-tk-luciferase in phenol red-free D-MEM/F-12 medium containing 10% (v/v) heat-inactived charcoal-stripped fetal bovine serum, 1% (v/v) Penicillin-Streptomycin, 2 mM glutaMax-1, 1 mM sodium pyruvate. The wells are then washed once with 150 μl of experimental medium. Finally, the cells are treated for 24 hr at 37° C. in replicates of 8 wells/treatment with 150 μl/well of vehicle (<0.1% v/v DMSO) or compound that is diluted >1000-fold into experimental medium. SKI-606 was tested in a dose response assay in the presence of 0.1 nM 17β-estradiol to stimulate ERE-luc.

After treatment, the cells are lysed on a shaker for 15 min with 25 μl/well of 1× cell culture lysis reagent (Promega Corporation). The cell lysates (20 μl) were transferred to a 96 well luminometer plate, and luciferase activity were measured in a MicroLumat LB 96 P luminometer (EG & G Berthold) using 100 μl/well of luciferase substrate (Promega Corporation).

Bosutinib as a single agent was used as a comparative example with regard to the combination of the invention.

### COMPARATIVE EXAMPLE 1

### ERα Levels in MCF7 Treated w/SKI (Src Kinase Inhibitor)

Day 1. Plate cells (MCF-7 cells were grown in 10% FBS (Gibco), DMEM/F12 (w/o phenol red), 1× Glutamax, 1× Pen-Strep, 1× Non-essential Amino Acids, 1× Sodium Pyruvate, 1× Hepes.), MCF-7 cells (P14) were plated at 400K per 6-well in 10% FBSS (Biosource), MEM (w/o phenol red), 1× Glutamax, 1× Pen-Strep, 1× Non-essential Amino Acids, 1× Sodium Pyruvate, 1× Hepes.
Day 2. Changed media to:
2% FBSS (Biosource), MEM (w/o phenol red), 1× Glutamax, 1× Pen-Strep, 1× Non-essential Amino Acids, 1× Sodium Pyruvate, 1× Hepes.
Day 3. Treatment for 24 hours was performed
Compounds used:

| No Treatment | | 0.2% DMSO |
|---|---|---|
| 10 | nM | 17β-Estradiol |
| 10 | nM | 17 β -Estradiol + 10 uM PP2 |
| 10 | nM | 17β -Estradiol + 10 uM SKI 606 |
| 1 | uM | ICI |
| 10 | uM | PP2 |
| 10 | uM | SKI 606 |

Treated cells with the following concentrations:

| 10 uM | 1 uM | 100 nM | 10 nM | 1 nM |
|---|---|---|---|---|
| (1 × 10⁻⁵) | (1 × 10⁻⁶) | (1 × 10⁻⁷) | (1 × 10⁻⁸) | (1 × 10⁻⁹) |

Made dilutions at 1:1000 (so DMSO was 0.1%), therefore concentrations used:

| | | | | |
|---|---|---|---|---|
| (1 × 10⁻²) | (1 × 10⁻³) | (1 × 10⁻⁴) | (1 × 10⁻⁵) | (1 × 10⁻⁶) |

Day 4. Harvested/lysed cells
Collected cells by direct lysis in 200 ul LDS (1×) with protease and phosphatase inhibitors

### Boiled Sample at 95 C for 4 min

### Stored at −80 C

Sonicated samples if necessary prior to loading on gel. Dual Src/AbI kinase inhibitor SKI-606 inhibits both genomic and non-genomic signaling by ER. In addition, SKI-606 provides additional benefit to tamoxifen in blocking proliferation of estrogen-dependent breast tumor cells.

### COMPARATIVE EXAMPLE 2

### In Vitro Data of Bosutinib as Single Therapy Regimen

A phase 1 single-agent trial of bosutinib to determine the safety, tolerability, and pharmacokinetics of orally administered bosutinib in subjects with advanced-stage, previously treated, solid tumors is currently ongoing. One-hundred and fifty-one (151) subjects have been treated. Subjects are continued on bosutinib until disease progression, or the occurrence of a significant toxicity considered possibly related to bosutinib. Dosing was started at 50 mg per day. Subsequent dose levels studied have included 100 mg, 200 mg, 300 mg, 400 mg, 500 mg and 600 mg. Safety is assessed for 21 days in 3 to 6 subjects before beginning the cohort at the next incremental dose. Dose limiting toxicities observed were nausea, vomiting, diarrhea, and rash. In this population of subjects with multiple-relapsed solid tumors, the protocol-defined maximum tolerable dose was 500 mg daily. Due to the emergence of early grade 2 gastrointestinal toxicities, however, the part 2 dose chosen was 400 mg for solid tumor subjects. Preliminary pharmacokinetic analyses demonstrated that after oral administration of bosutinib with food, absorption was relatively slow with median tmax of appropriately 3 to 6 hours. Mean steady state Cmax and AUC values were 157 ng/mL and 2581 ng·h/mL for the 400-mg dose group, respectively, and 200 ng/mL and 3660 ng·h/mL for the 500-mg dose group, respectively and 208 ng/mL and 3360 ng·h/mL for the highest dose of 600 mg, respectively. Multiple-dose exposure was approximately 2 to 3-fold higher than the single-dose exposure. The mean elimination half-life for bosutinib was approximately 22 to 25 hours. The variability (CV) for Cmax and AUC ranged from 6 to 76%.

### COMPARATIVE EXAMPLE 3

### Single-Agent Bosutinib Data in Subjects with Metastatic Breast Cancer

In a phase 2, open-label, study of bosutinib in subjects with advanced or metastatic breast cancer where bosutinib is given at 400 mg once-daily, 73 total patients were randomized. As of 9 Jun. 2008 snapshot, of 73 patients, 4 subjects had a partial response and 1 near response (29% tumor shrinkage) were observed. All of the responding patients had ER/PgR+ metastatic disease, but were less heavily pretreated with chemotherapy than typical patients and none had received prior endocrine therapy. In addition a clinical benefit of 27.4% was observed with on top of responders, stable disease ≧24 weeks reported on 13 patients, out of whom at least 6 were identified as ER/PgR+. In that trial, the median PFS was 15 weeks for all patients, 15.6 weeks and 14.9 weeks in ER+ and/or PgR+ and ER+ and/or PgR+, HER2-patients subgroups, respectively. There were 51 ER/PgR+ patients enrolled in the trial and a little more than half had received prior endocrine therapy, out of them 33 patients were ER/PgR+, HER2-. The preliminary safety data available for 73 subjects as of 9 Jun. 2008 are consistent with data from other ongoing studies. TEAEs were most frequently associated with gastrointestinal disorders (diarrhea, 66%; nausea, 55%; vomiting, 47%; abdominal pain, 12%; dyspepsia, 10%), general disorders (fatigue, 26%, asthenia, 19%, peripheral oedema, 11%; pyrexia, 10%), infections and infestations (16%), metabolism disorders (ALT increase, 12%; AST increase, 12%, weight loss, 11%; anorexia, 18%), musculoskeletal disorders (anthralgia, 12%; back pain, 12%), headache (14%), and rash (14%). Out of all of these TEAEs, only gastrointestinal disorders (diarrhea, 64%; nausea, 51%; vomiting, 42%; abdominal pain, 10%), general disorders (fatigue, 16%, asthenia, 14%), metabolism disorders (ALT increase, 11%; AST increase, 10%; anorexia, 15%) and rash (10%) were considered at least possibly related to bosutinib.

### Example 1

### Biomarkers for Bosutinib Sensitivity and Resistance

Patients with the same histological subtypes of breast cancer do not all respond to the same treatments indicating that heterogeneity of gene and protein expression exists in tumors resulting in differential responses to treatment. Sub-classification of tumors by ER, PgR and HER2 status increases response rates; however, a significant fraction of patients are resistant to targeted treatments despite expressing these biomarkers of sensitivity. An approach that yields more predictive biomarkers of sensitivity and resistance to treatments is desired. To that end, validation of pre-clinically derived biomarkers of sensitivity and resistance to bosutinib is evaluated. Specifically, four candidate biomarkers of patient selection were identified in the preclinical setting for bosutinib. These are divided into 2 general sets of markers, those based on preclinical modeling of sensitivity and resistance to bosutinib and those based on Src signaling pathway activation. Resistance biomarkers include a set of genes and proteins identified as having high differential expression in sensitive and resistant tumors in an animal model of pancreatic cancer (bosutinib genomic resistance and bosutinib proteomic resistance biomarkers, respectively). In related preclinical studies, 2 biomarkers were identified that are based on the oncogenic Src signaling pathway, one at a protein level and one at an RNA level (Src pathway proteomic biomarker and Src pathway genomic biomarker, respectively). Each of these four biomarkers is assessed in each patient (where adequate tissue is available) with the intention of prospectively validating the predictability of the biomarker to exclude resistant patients. In the event that none of the pre-clinically derived candidate biomarkers accurately predicts resistant patients, biomarker of patient selection is derived within the context of the clinical trial. Toward this end, global RNA and signaling protein profiles are obtained from each patient's sample. Tumor samples from the primary (or an alternate, if the primary is not available) formalin-fixed paraffin embedded tumor tissue block collected at screening will be assessed for proteomic markers of bosutinib sensitivity and resistance (including, but not limited to, phosphor-Src, p130cas, Paxillin, FAK, phosphor-STAT5, phosphor-STAT6 and phosphor-AMPK). Additionally, RNA expression pattern biomarkers of sensitivity and resistance is assessed. Five micron sections are cut from a paraffin embedded block of tumor and placed on glass slides. Protein is extracted from at least 6 of these slides and protein expression will be assessed by reverse phase protein array (including, but not limited to phosphor-STAT5, phosphor-STAT6 and phosphor-AMPK). Protein expression is also assessed by IHC staining using AQUA technology of at least 4 of these slides (including, but not limited to, phosphor-Src, p130cas, FAK and Paxillin). AQUA is a technology that allows quantitative assessment of IHC staining. RNA is extracted from at least 10 slides. RNA expression will be assessed by TaqMan Low Density Arrays for a defined set of resistance genes. Global RNA profiling is performed by gene expression microarray analysis (Affymetrix chips) using the same RNA samples.

### Example 2

### Cell Proliferation Inhibition Using Bosutinib and Tamoxifen

Cell proliferation assays were performed in two formats; a three day proliferation assay in fully supplemented medium with phenol red, and a 7 day proliferation assay in phenol red free medium supplemented with charcoal-stripped medium. Data are summarized in Table 1. ER-positive MCF7 and MDA-MB-361 cells showed an improved response to SKI-606 in stripped medium. While MCF7 cells appear to be more sensitive to SKI-606 in the absence of estrogen, the cells did not grow well under these conditions, rendering the data less reliable than when estrogen was present. ER positive T47D cells were slightly more sensitive to SKI-606 in the presence of estrogen. BT20 cells were equally sensitive to SKI-606 in stripped serum medium, regardless of whether estrogen was present.

TABLE 1
Proliferation Data for WAY-173606
in Various Breast Tumor Lines

IC50 (uM)

| | no E2 | E2 | 10% FCS |
|---|---|---|---|
| BT-20 (ER neg) | 0.28 | 0.24 | |
| BT474 (ER neg) | 2.22 | 2.32 | |
| MDA-MB-231 (ER neg) | 5.10 | 5.05 | 4.3 |
| MDA-MB-361 | 0.74 | 0.38 | 2.5 |
| MCF7 | 0.08 | 0.86 | 6.5 |
| SKBR3 (ER neg) | 1.43 | 1.38 | 1.5 |
| T47D | 2.85 | 1.28 | |
| MDA-MB-468 (ER neg) | | | 5 |
| MDA-MB-435 (ER neg) | | | 3.4 |
| | 7 Day assay | | 3 Day assay |

Proliferation data for SKI-606 in various breast tumor lines in complete medium (10% FCS) and charcoal stripped serum with no phenol red (no E2, E2) with and without 10 nM estrogen. The complete medium data were obtained in 3-day assays, while stripped serum assays were extended for 7 days with one change of medium at day 5. The IC50 values for the BT20 line was calculated using the “plateau” cell number evident at 5 μM SKI-606.

MCF7 and MDA-MB-361 cells were selected for further study. The effect of 4-hdroxytamoxifen on proliferation in the presence and absence of estrogen were examined. Proliferation of both cell lines was stimulated by low concentrations of estrogen, and could be inhibited in a dose-dependent manner by 4-hydroxytamoxifen.

The effect of SKI-606 on estrogen-independent proliferation in the presence and absence of 4-hydroxytamoxifen. Results indicated that SKI-606 inhibited estrogen-dependent and estrogen-independent growth in both cell lines, and when combined with 4-hydroxytamoxifen, reduced proliferation further in MCF7 cells.

Several concentrations of estrogen were examined, but no difference in the effect of SKI-606 was observed in the concentration range employed (1 uM to 0.15 nM E2). Tamoxifen (4-hydroxytamoxifen) treatment inhibits estrogen-independent MCF7 cell proliferation, but has little effect on MDA-MB-361 cell proliferation in the absence of estrogen. MDA-MB-361 cells were less affected by tamoxifen treatment, and the combination showed an improvement over SKI-606 alone.

### Example 3

### Anti-Tumor Activity of Bosutinib and Exemestane Combination

Subjects with solid tumors are enrolled in each dose group of the combination of bosutinib and exemestane. Each subject participates in only 1 dose group. For the purpose of this study, a cycle is defined as a 21-day period. Each subject participates at only 1 dose level. Subjects receive oral bosutinib tablets (160 or 240 mg) daily in combination with oral exemestane (750 or 1000 mg/m² BID (twice daily)) on days 1-14 of a 21-day cycle (no capecitabine administered days 15-21). For comparative purposes, bosutinib as a single agent is administered orally once daily continuously according to the clinical protocol.

Based on the preclinical information summarized above supporting the combination of a Src inhibitor with endocrine therapy and evidence of anti-tumor activity of bosutinib in ER+breast cancer, it is useful evaluate the safety and efficacy of bosutinib plus exemestane versus exemestane alone in postmenopausal women with second-line or resistant ER+ breast cancer.

### Example 4

### Combination of Bosutinib and Exemestane in Metastatic Breast Cancer

A clinical regimen is developed to compare the efficacy, in terms of progression free survival (PFS) assessed by an independent radiology vendor, of bosutinib in combination with exemestane versus exemestane alone as second line treatment for ER+/PgR+/HER2-advanced or metastatic breast cancer in postmenopausal women. Other clinical objectives for the pharmaceutical combination include, but are not limited to for example, to evaluate the safety profile of bosutinib in combination with exemestane, to evaluate the population pharmacokinetics (PK) of both bosutinib and exmestane combination, and exemestane alone, to evaluate additional efficacy parameters such as objective response rate (ORR), overall survival (OS) at 2 years, duration of response, and PFS assessed by investigational sites, to examine the health-related quality of life (HRQoL) of bosutinib in combination with exemestane versus exemestane alone, to validate pre-clinically derived candidate biomarkers of sensitivity and resistance to bosutinib and to derive and validate biomarkers of sensitivity and resistance to bosutinib in combination with exemestane in the event that the pre-clinically derived biomarkers do not accurately predict sensitivity and/or resistance.

### Example 5

### Bosutinib and Exemestane in Combination for ER+ (HER2-) Metastatic Breast Cancer

Patients having diagnosed metastatic breast cancers are treated using a regimen of bosutinib and exemestane for three 28-day cycles. Control groups include patients who receive oral bosutinib monotherapy (240 mg daily) (Group 1) or a combination of Bosutinib [oral, 750 mg daily] and exemestane [Aromasin, oral 25 mg daily] (Group 2) according to manufacturer recommendations as a comparative. Patients receive oral bosutinib tablets (either 160 mg or 240 mg) daily in combination with oral exemestane (either 25 mg or 50 mg twice daily) on days 1-14 of a 21-day cycle (no capecitabine administered days 15-21). Oral dosing of bosutinib begins at cycle 1 and continues on the remaining days of the each cycle. It is anticipated that results will show that bosutinib in combination with exemestane will significantly improve objective response rate as compared to the combination of lapatinib and capecitabine and/or prolonged subject's time to tumor progression (TTP) when compared to capecitabine monotherapy. It is further anticipated that side effects will be minimized as compared to the combination of lapatinib and capecitabine, in view of the lower effective dosages permitted by the combination of bosutinib and exemestane.

### Example 6

### Bosutinib and Exemestane Combination

Part 1 is a safety lead-in phase, intended to confirm the tolerability of the dose of bosutinib to be used in the combination arm during the randomized period of the study, initially selected as 400 mg bosutinib daily. An initial cohort of 10 subjects are enrolled and evaluated for safety. Subjects receive 400 mg bosutinib and 25 mg exemestane daily, and are monitored for 28 days for adverse events and dose-limiting toxicities (DLTs). The total sample size for part 1 depends on the observed DLT rate in subjects treated with the combination regimen. If no safety concerns arise (see section 18.0, and protocol for details), all following eligible subjects are enrolled into the randomized part 2 of the study. If safety criteria, defined as calculated DLT rate, are not sufficient to begin part 2, an additional 10 subjects are then enrolled at same or lower dose of bosutinib. Up to 30 patients may be enrolled in part 1. The decision of whether to de-escalate the bosutinib dose or begin the randomized period of the study is based on the overall toxicity profile of the current combination regimen.

Adverse events and DLTs are assessed from the first dose of study drug through day 28 of month 1 in part 1. A DLT is defined as any of the following events, related to treatment with bosutinib and/or exemestane, and observed during the safety lead-in phase of the study: Any grade 3 or 4 nonhematologic toxicity (grade 3 asthenia is not considered a DLT unless lasting >3 days. Grade 3 nausea, vomiting or diarrhea will NOT be considered DLTs UNLESS subject is already receiving optimal medical therapy), Grade 4 neutropenia lasting days or grade 4 febrile neutropenia, Grade 4 thrombocytopenia lasting ≧2 days or associated with bleeding or requiring platelet transfusion and Delayed recovery (to ≧NCI CTCAE grade 1 or baseline) from 1 of the above listed toxicities that are related to bosutinib and/or exemestane that delays the initiation of the next dose by more than 3 weeks. Toxicity will be graded according to the National Cancer Institute (NCI) Common Terminology Criteria for Adverse Events, version 3.0.

In part 2, a total of approximately 164 postmenopausal subjects with locally advanced or metastatic ER and/or PgR positive, HER2 negative breast cancer is randomly assigned between the following 2 treatment arms:

Arm A: exemestane+bosutinib (dose determined in Part 1)
Arm B: exemestane (25 mg daily)

Cross-over of subjects from arm B to arm A is permitted at the discretion of the investigator, if progression is confirmed radiologically in arm B (per site assessment). Subjects enrolled in both parts participate in the study for approximately 25 months. This includes 28 days for screening, approximately 6 months for treatment, and 24 months follow-up (from first dose of test article). This study is completed in approximately 39 months. This includes 3 months for part 1, approximately 12 months for accrual of part 2, approximately 6 months for active treatment, and 24 months follow-up for survival (from first dose of test article). The end of the study is when the last subject has completed 24 months follow-up for survival or when the last subject has expired, whatever occurs first. Approximately 194 subjects participate in this study at approximately 75 sites globally with approximately 2 to 3 subjects at each site. This number may vary based on enrollment capabilities of each site. Approximately 10 to 30 subjects are enrolled in part 1 of the study (safety lead-in). The actual number of subjects enrolled depends on the tolerability of bosutinib combined with exemestane and on the observed DLT rate in subjects treated with that combination. In part 2, approximately 164 subjects are randomized equally between the 2 treatment arms. Subjects enrolled in part 1, and withdrawn from the study for a reason other than a DLT, may be replaced. Subjects enrolled in part 2 who withdraw from the study are not replaced, regardless of the reason for withdrawal.

Inclusion criteria
1. Women aged 18 years or older.
2. Confirmed pathologic diagnosis of breast cancer.
3. Locally advanced, metastatic, or loco-regional recurrent and not amenable to curative treatment with surgery or radiotherapy.
4. Subjects must be postmenopausal, as defined by at least one of the following:
   Women ≧60 yrs
   Women 45 to 59 years of age with spontaneous amenorrhea for >12 months prior to treatment day 1
   Prior bilateral oophorectomy
   Ovarian ablation by radiotherapy confirmed by FSH level in the postmenopausal range.
   Women 45 to 59 years of age with cessation of menses duration <12 months or secondary to hysterectomy AND with FSH levels before treatment day 1 above the lower limit of normal for laboratory postmenopausal range (or >34.4 IU/L if institutional range is not available)
   Women of age 45-59 yrs previously on HRT who discontinued HRT at breast cancer diagnosis and who have FSH level prior anti-cancer therapy or treatment day 1 clearly in the laboratory postmenopausal range (or >34.4 IU/L if institutional range is not available)
5. Documented ER+ and/or PgR+ and erbB2-tumor based on most recently analyzed biopsy, as documented by a local laboratory. (In case of more than one result, receptor status from the most recent biopsy should be used.)
6. Availability of either formalin-fixed paraffin embedded tumor tissue block or unstained slides for central review of ER/PgR/erbB2 receptor status and exploratory biomarker analysis of bosutinib sensitivity/resistance.
7. Progression of locally advanced or metastatic disease during treatment with a non-steroidal AI or tamoxifen, or progression during treatment with (or within 6 months of discontinuation of) an adjuvant non-steroidal AI.
8. At least 1 radiologically measurable lesion as defined by response evaluation criteria in solid tumors (RECIST).
9. Eastern Cooperative Oncology Group (ECOG) status of 0 to 2, not declining within 2 weeks before inform consent signing
10. Recovery from all clinically significant adverse effects related to prior therapies (excluding alopecia).
11. Left ventricular ejection fraction (LVEF) within institutional limits of normal.
12. Screening laboratory values within the following parameters:
   Absolute neutrophil count (ANC): >1.5×10⁹/L (1500/mm³)
   Platelet count: ≧75×10⁹/L (75,000/mm³)
   Hemoglobin: ≧9 g/dL (90 g/dL)
   Serum creatinine: ≦1.5× upper limit of normal (ULN)
   Total bilirubin: ≦1.5×ULN
      ALT and AST: ≦2.5×ULN (or ≦5 if liver metastases are present).

### Exclusion Criteria

1. Prior exemestane, prior bosutinib, or any other prior anti-Src therapy
2. More than 1 prior endocrine treatment for locally advanced or metastatic breast cancer
3. More than 1 prior cytotoxic chemotherapy regimen in metastatic setting
4. Subjects with bone or skin as the only site of disease
5. Major surgery, chemotherapy, radical (curative intent) radiotherapy, any investigational agents, or other cancer therapy within 14 days of treatment day 1.
6. Presence of life-threatening metastatic disease defined as extensive (more than one-third of the organ) hepatic involvement, or symptomatic pulmonary lymphangitic spread more than one-half of the organ).
7. Active central nervous system (CNS) metastases, as indicated by clinical symptoms, cerebral edema, and/or progressive growth. Subjects with a history of CNS metastases or cord compression are allowable if they have been definitively treated, have been clinically stable for at least three months, and off anticonvulsants before first dose of test article (steroids are permitted, provided subject is on stable doses).
8. Known or suspected allergy to bosutinib, exemestane, or excipients
9. Inability or unwillingness to swallow tablets or capsules.
10. QTc interval >0.47 second or known history of QTc prolongation or Torsade de Pointes (TdP).
11. History of clinically significant or uncontrolled cardiac disease including:
   history of or active congestive heart failure
   uncontrolled angina or hypertension within 3 months
   myocardial infarction (within 12 months)
   clinically significant ventricular arrhythmia (such as ventricular tachycardia, ventricular tachycardia, ventricular fibrillation, or Torsades de pointes).
   diagnosed or suspected congenital or acquired prolonged QT syndrome
   history of prolonged QTc
   unexplained syncope
12. Any other cancer within 5 years prior to screening with the exception of adequately treated cervical carcinoma in situ, or adequately treated basal or squamous cell carcinoma of the skin.
   Any major illness/condition that, in the investigator's judgment, substantially increases the risk associated with the subject's participation in the study, or could preclude the evaluation of the subject's response. Examples include, but are not limited to, serious active infection (ie requiring intravenous antibiotic or antiviral agent), uncontrolled major seizure disorder, significant pulmonary disorder (eg interstitial pneumonitis, pulmonary hypertension), or psychiatric disorder that would interfere with subject safety or informed consent. Tumor assessment (see Radiographic evaluation chart) is evaluated. The same method of measurement should be used for the same subject throughout the duration of the study.

### Test Article and Administration

Test article refers to either bosutinib and/or exemestane. Test article is administered only to subjects who have provided informed consent. Once test article has been assigned to a subject, it must not be reassigned to another subject.

Part 1: Subjects receive: 400 mg of bosutinib (corresponding to 4 (4×100 mg) tablets) once daily, by mouth with food, preferably in the morning and, 25 mg exemestane, preferably after a meal [refer to Aromasin® prescribing information]. Exemestane is taken preferably in the morning 30 minutes to 1 hour after bosutinib intake. Each subject receives daily test article during the active phase of the study until disease progression, unacceptable toxicity, or withdrawal of consent occurs.

Part 2: Subjects are randomized to 1 of the following arms:

Arm A: Subjects receive concomitantly 400 mg of bosutinib once daily, by mouth with food, preferably in the morning and 25 mg exemestane, preferably after a meal (referenced to the prescribing information).
Arm B: Subjects receive 25 mg exemestane once daily, by mouth, preferably after a meal (please refer to the prescribing information).
Cross-over of subjects from arm B to arm A is permitted at the discretion of the investigator, if progression is confirmed radiologically in arm B (per site assessment).

A flowchart for survival follow-up and radiographic evaluation chart for tumor assessments is prepared. Evaluation of tumor response in this study will be based on modified RECIST criteria. Primary analyses of the efficacy endpoints are based on tumor response assessed by an independent radiology vendor. Interim efficacy analysis will be based on tumor assessments by investigators. Clinical activity of the combination is obtained by performing tumor assessments for all subjects at screening, and then every 2 months (8 weeks) throughout the course of the study, regardless of treatment schedule. Subjects considered evaluable for efficacy have completed at least one follow-up radiographic tumor assessment approximately 2 months (8 weeks) after starting treatment, have received at least 2 weeks of bosutinib and exemestane (arm A), and at least 2 weeks of exemestane alone (arm B), and have reported no major protocol violations. As soon as evaluations for each tumor assessment are completed, the investigator assesses the subject response based on criteria defined in Response Criteria. Measurable (target) lesions and all nonmeasurable (non-target) lesions are documented at baseline and followed, according to the Documentation of Target and Nontarget lesion protocol. All lesions should be followed with the same method of assessment throughout the study. In the case of spiral (helical) CT, the reconstruction slice interval (or thickness) should be ≦55 mm cuts. If conventional (axial) CT is used, contiguous images should be acquired at 8 mm or less. Enhanced MRIs (with ≦5 mm reconstruction intervals) may be used instead of CT scans in cases of CT contrast media allergy or renal insufficiency (creatinine >2 mg/dL [176.8 mmol/L]), but the same method of assessment is used throughout the course of the study. Superficial lesions, such as skin nodules and palpable lymph nodes, are considered measurable if objective documentation can be provided, if measurable on scan, or as a color photograph with a ruler; if this documentation is not provided, such lesions should be followed up as nontarget lesions.

The clinical regimen in the Example is practiced over a time period of 39 months: 3 months for part 1 incl. 4 weeks accrual and 36 months for part 2 incl. 12 months accrual and 24 months for subject participation.

Timed blood samples (3.0 mL) are collected into potassium (K2)-EDTA treated tubes for analysis of bosutinib and its metabolite plasma concentrations. Timed blood samples (3.0 mL) are also collected into potassium (K2)-EDTA treated tubes for analysis of exemestane. Samples are taken at suitably determined time-points outlines in collection schedules. Curves of bosutinib and its metabolite concentration vs. time in plasma, as well as exemestane plasma concentration vs. time are constructed for each subject and analyzed by non-compartmental PK methods. Among the parameters to be estimated are the observed maximum concentration, time of maximum concentration, and AUC (0-24). If appropriate, other analyses may be undertaken. Formalin-fixed paraffin embedded tumor tissue block/slides are available for preparation of specimens and analysis/review of hormone receptor status and HER2 expression assessed by suitable clinical protocols, e.g. IHC and FISH.

### Example 7

### Bosutinib in Combination with Letrozole

Part 1 is a safety lead-in phase, intended to confirm the tolerability of the dose of bosutinib to be used in the combination arm during the randomized period of the study, initially selected as 400 mg bosutinib daily. An initial cohort of 10 to 60 subjects are enrolled and evaluated for safety. Subjects receive 400 mg bosutinib and 2.5 mg letrozole daily, and are monitored for 28 days for adverse events and dose-limiting toxicities (DLTs). The total sample size for part 1 depends on the observed DLT rate in subjects treated with the combination regimen. If no safety concerns arise, all following eligible subjects are enrolled into the randomized part 2 of the study. They will either receive 400 mg bosutinib (or as determined in part 1) combined with 2.5 mg letrozole or 2.5 mg letrozole alone daily until radiologically determined disease progression. Subjects who have completed the treatment phase of the study will undergo follow up every 12 weeks for survival status for 3 years. Subjects who have until radiologically determined disease progression on the letrozole-alone arm will be allowed to cross over to the combination of bosutinib and letrozole. In case of an adverse event, the dose of bosutuinib may be lowered to 300 mg daily and held at that level for 3 weeks.

In part 2, a total of approximately 160 to 250 postmenopausal subjects with locally advanced or metastatic ER and/or PgR positive, HER2 negative breast cancer is randomly assigned between the following 2 treatment arms:

Arm A: 400 mg bosutinib+2.5 mg leterozole (dose determined in Part 1)
Arm B: letrozole (2.5 mg daily)

To be evaluable for efficacy, a subject must have had a baseline scan, completed at least 1 follow-up radiographic tumor assessment approximately 2 months (8 weeks) after starting treatment, have received at least 2 weeks of bosutinib and letrozole (arm A), and at least 2 weeks of letrozole alone (arm B), and have reported no major protocol violations. Contrast enhanced computed tomographic (CT) or magnetic resonance imaging (MRI) scans will be performed during screening, and then every 8 weeks until disease progression. Scans will continue until disease progression if a subject discontinues the treatment for a reason other than disease progression. All scans will be sent to radiology for independent response assessment. RECIST criteria will be used to assess tumor response and determine disease progression. Functional Assessment of Cancer Therapy for breast cancer (FACT-B) will be performed at baseline, week 12, week 24, week 52, and at the end of treatment. (Part 2-arm A only).

For determination of bosutinib, bosutinib metabolite, and letrozole plasma concentrations, all subjects in part 2-arm A (bosutinib combined with letrozole) will provide 2 predose blood samples (3 mL each) on day 1 (sampling time “0”). Two (2) timed blood samples will be collected at 2, 3, 4, 6, 8 and 24 hours on day 29 (week 5, day 1) after once-daily oral administration of bosutinib and letrozole. If the subject receives less than 4 to 5 consecutive days of bosutinib and/or letrozole treatment before day 1 of week 5, PK sampling will be postponed until day 1 of month 3. Pharmacokinetic profiles of bosutinib, its metabolites and letrozole will be evaluated using compartmental or non-compartmental analyses method.

Formalin-fixed paraffin embedded tumor sample (block or a set of slides) preferably representing the tumor specimen before any systemic therapy, will be collected from all subjects and analyzed for hormone receptor status, erbB2 expression, mRNA expression, and protein biomarker assays (tissue microarray) that may predict response to bosutinib or to the combination of bosutinib and letrozole (eg, p-Src, FAK, Paxillin, p130cas).

The clinical regimen in the Example is practiced over a time period of 39 months: 3 months for part 1 including 4 weeks accrual and 36 months for part 2 including 12 months accrual and 24 months for subject participation.

Timed blood samples (3.0 mL) are collected into potassium (K2)-EDTA treated tubes for analysis of bosutinib and its metabolite plasma concentrations. Timed blood samples (3.0 mL) are also collected into potassium (K2)-EDTA treated tubes for analysis of letrozole. Samples are taken at suitably determined time-points outlines in collection schedules. Curves of bosutinib and its metabolite concentration vs. time in plasma, as well as letrozole plasma concentration vs. time are constructed for each subject and analyzed by non-compartmental PK methods. Among the parameters to be estimated are the observed maximum concentration, time of maximum concentration, and AUC (0-24). If appropriate, other analyses may be undertaken. Formalin-fixed paraffin embedded tumor tissue block/slides are available for preparation of specimens and analysis/review of hormone receptor status and HER2 expression assessed by suitable clinical protocols, e.g. IHC and FISH.

1. A pharmaceutical composition comprising: a therapeutically effective amount of a Src kinase inhibitor, or a pharmaceutically acceptable salt thereof, a therapeutically effective amount of an aromatase inhibitor, or a pharmaceutically acceptable salt thereof, and a pharmaceutically acceptable carrier.

2. The pharmaceutical composition according to claim 1, wherein the Src inhibitor is selected from 4-(2,4-dichloro-5-methoxy-phenylamino)-6-methoxy-7-[3-(4-methyl-piperizin-1-yl)-propoxy]-quinoline-3-carbonitrile (bosutinib), (E)-N-{4-[3-chloro-4-(2-pyridinyl methoxy) anilino]-3-cyano-7-ethoxy-6-quinolinyl}-4-(dimethylamino)-2-butenamide(neratinib), N-(2-chloro-6-methylphenyl)-2-[[6-[4-(2-hydroxyethyl)-1-piperazinyl]-2-methyl-4-pyrimidinyl]amino]-5-thiazole carboxamide monohydrate, (PP1), (PP2), (AP23464), (PD166326) and pharmaceutically acceptable salts thereof.

3. The pharmaceutical composition according to claim 2, wherein the aromatase inhibitor is selected from: anastrozole, letrozole, vorazole, tamoxifen and exemestane or a pharmaceutically acceptable salt thereof.

4. The pharmaceutical composition according to claim 1 wherein the Src inhibitor is bosutinib, or a pharmaceutically acceptable salt thereof, and the aromatase inhibitor is exemestane, or a pharmaceutically acceptable salt thereof.

5. The pharmaceutical composition according to claim 1 wherein the Src inhibitor is bosutinib, or a pharmaceutically acceptable salt thereof, and the aromatase inhibitor is letrozole, or a pharmaceutically acceptable salt thereof.

6. The pharmaceutical composition according to claim 1 wherein the Src inhibitor is bosutinib, or a pharmaceutically acceptable salt thereof, and the aromatase inhibitor is tamoxifen, or a pharmaceutically acceptable salt thereof.

7. A method of treating disease states associated with angiogenesis and/or vascular permeability by administering to a patient a combination of a Src kinase inhibitor, or a pharmaceutically acceptable salt thereof, and an aromatase inhibitor, or a pharmaceutically acceptable salt thereof, wherein the Src kinase inhibitor and the aromatase inhibitor are as defined in claim 1.

8. A method of treating mammalian diseases associated with a non-receptor tyrosine kinase by administering to a patient a combination of a Src kinase inhibitor, or a pharmaceutically acceptable salt thereof, and an aromatase inhibitor, or a pharmaceutically acceptable salt thereof, wherein the Src kinase inhibitor and the aromatase inhibitor are as defined in claims 1.

9. The method according to claim 8, wherein the disease associated with the non-receptor tyrosine kinase is a cancer selected from breast, kidney, bladder, thyroid, mouth, larynx, esophagus, stomach, colon, ovary, lung, pancreas, skin, liver, prostate and brain cancer.

10. The method according to claim 9, wherein the disease associated with the non-receptor tyrosine kinase is breast cancer.

11. The method according to claims 8 further comprising administering an additional chemotherapeutic agent.

12. The method according to claim 8 further comprising radiation.

13. The method according to claim 7, wherein the Src inhibitor and the aromatase inhibitor are administered simultaneously to the patient.

14. The method according to claim 7, wherein the Src inhibitor is administered to the patient prior to administering the aromatase inhibitor.

15. The method according to claim 7, wherein the aromatase inhibitor is administered to the patient prior to administering the Src inhibitor.

16. The method according to claim 7, wherein the amounts of the Src kinase inhibitor and the aromatase inhibitor are such that the combined therapeutic effect is synergistic.

17. The method according to claim 7, wherein the amounts of the Src kinase inhibitor and the aromatase inhibitor are subtherapeutic.

18. The method according to claim 7, wherein the amount of aromatase inhibitor that is administered is from about 1 mg/day to about 100 mg/day.

19. The method according to claim 7, wherein the amount of the aromatase inhibitor that is administered is from about 1 mg/day to about 50 mg/day.

20. The method according to claim 7, wherein the amount of Src inhibitor that is administered is from about 100 mg/day to about 1000 mg/day.

21. The method according to claims 7, wherein the amount of Src inhibitor that is administered is from about 100 mg/day to about 750 mg/day.

22. A pharmaceutical pack for treating a neoplasm in one individual mammal, said pharmaceutical pack comprising: (a) at least one unit dose of an aromatase inhibitor; and (b) at least one unit dose of an Src inhibitor.

23. The pharmaceutical pack according to claim 22 comprising: (a) at least one unit dose of an aromatase inhibitor selected from anastrozole, letrozole, vorazole, tamoxifen and exemestane or a pharmaceutically acceptable salt thereof; and (b) at least one unit dose of an Src inhibitor selected from 4-(2,4-dichloro-5-methoxy-phenylamino)-6-methoxy-7-[3-(4-methyl-piperizin-1-yl)-propoxy]-quinoline-3-carbonitrile, (E)-N-{4-[3-chloro-4-(2-pyridinyl methoxy) anilino]-3-cyano-7-ethoxy-6-quinolinyl}-4-(dimethylamino)-2-butenamide, N-(2-chloro-6-methylphenyl)-2-[[6-[4-(2-hydroxyethyl)-1-piperazinyl]-2-methyl-4-pyrimidinyl]amino]-5-thiazole carboxamide monohydrate, PP1, PP2, AP23464, PD166326 or a pharmaceutically acceptable salt thereof.

24. A pharmaceutical composition as defined in claim 1 for use as a medicament.

25. A pharmaceutical composition as defined in claims 1 for use in treating cancer.

26. A product comprising a therapeutically effective amount of a Src kinase inhibitor, or a pharmaceutically acceptable salt thereof, and a therapeutically effective amount of an aromatase inhibitor, or a pharmaceutically acceptable salt thereof, wherein the Src kinase inhibitor and the aromatase inhibitor are as defined in claim 1, as a combined preparation for simultaneous, separate or sequential use in the treatment of cancer.
